SEQUENCE LISTING (57 sequences)

SEQ ID NO: 1 - DNA, 48 bp, synthetic construct
  source 1..48: mol_type = other DNA
cgcatatgatacgtatgccaataaccataaataattttagatatagtg

SEQ ID NO: 2 - DNA, 38 bp, synthetic construct
  source 1..38: mol_type = other DNA
cggctagcttaatcatttgtccatccttcatctgtagg

SEQ ID NO: 3 - DNA, 200 bp, synthetic construct
  source 1..200: mol_type = other DNA
gtgtagtagcctgtgaaataagtaaggaaaaaaaagaagtaagtgttatatatgatgattattttgtagatgtagataggataatagaatccatagaaaatataggttatacagttatataaaaattactttaaaaattaataaaaacatggtaaaatataaatcgtataaagttgtgtaatttttaaggaggtgtgtta

SEQ ID NO: 4 - DNA, 150 bp, synthetic construct
  source 1..150: mol_type = other DNA
tttttaacaaaatatattgataaaaataataatagtgggtataattaagttgttagagaaaacgtataaattagggataaactatggaacttatgaaatagattgaaatggtttatctgttaccccgtatcaaaatttaggaggttagtt

SEQ ID NO: 5 - DNA, 990 bp, synthetic construct
  source 1..990: mol_type = other DNA
ggttctcttaatttagtaatatcaatataaaaaactaaaagtacgttctttggagaaaaaacgtacttattaaatttatttgcaaactatattaatatacacatcctcggtaatatatcaatacaatacatgtaattaataatgttttttgtaaaagtttaagcttagtggtttactttcaaaatataataaaacgttaattatagaaaagtgaaagtaaggtgatgcttatggaaaatctatttgtaaaaattaagacattaaaaaataataataaagaatttgaagtaattattatacatttcaaaaatactgtaaatatacttattcgaaagtataatttagaaagtcattataatgatatcttatatcatttatggaatatacttaaaaaaattgatttgaataaatttaatacagaaaatgatttacacaaatatattagtagatctttaaaaagatattgcttagatatttgtaataaaaaaaatagggataaaaaaataatatataactcagaaattacaaatataaaattaaatttaatggaaaatagttgctcaaattatttaaacttcgaatttaatgatttaatatccatattacctgaaaatcaaagaaaaattttatatatgaaattttttgaagatatgaaagaatgtgatatagctaaaaagcttcacatgagtcgtcaagctgtatataaaaataaggttttagcattaaaaaaattagaacctatagtaaataaattaattaatatatagtttttataatttaattatgaataatattcttaagataaaaagtaaatttttaaaaatttaaattttcagtttacaaaaaataacctgattatgttatatgtaattgtaaaaaacatataaaaaatcagaaaaatttaggaggtatattattaatggattaaataataattttttaatttacttttgattaataaatattaaatgtttattttaattaggagca

SEQ ID NO: 6 - DNA, 624 bp, synthetic construct
  source 1..624: mol_type = other DNA
atggtatttgaaaaaattgataaaaatagttggaacagaaaagagtattttgaccactactttgcaagtgtaccttgtacctacagcatgaccgttaaagtggatatcacacaaataaaggaaaagggaatgaaactatatcctgcaatgctttattatattgcaatgattgtaaaccgccattcagagtttaggacggcaatcaatcaagatggtgaattggggatatatgatgagatgataccaagctatacaatatttcacaatgatactgaaacattttccagcctttggactgagtgtaagtctgactttaaatcatttttagcagattatgaaagtgatacgcaacggtatggaaacaatcatagaatggaaggaaagccaaatgctccggaaaacatttttaatgtatctatgataccgtggtcaaccttcgatggctttaatctgaatttgcagaaaggatatgattatttgattcctatttttactatggggaaatattataaagaagataacaaaattatacttcctttggcaattcaagttcatcacgcagtatgtgacggatttcacatttgccgttttgtaaacgaattgcaggaattgataaatagttaa

SEQ ID NO: 7 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 8 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHDAKFIDSLQENEFRLYYYNKFKDVASTLNKAKSIIGTTASLQYMKNVFKEKYLLSEDTSGKFSVDKLKFDKLYKMLTEIYTEDNFVNFFKVINRKTYLNFDKAVFRINIVPDENYTIKDGFNLKGANLSTNFNGQNTEINSRNFTRLKNFTGLFEFYKLLCVRGIIPFKTKSLDEGYNKALNDLCIKVNNWDLFFSPSEDNFTNDLDKVEEITADTNIEAAEENISLDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAFMFLNWAEELVYDFTDETNEVTTMDKIADITIIVPYIGPALNIGNMLSKGEFVEAIIFTGVVAMLEFIPEYALPVFGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLVLQVDRLKDEVNNTLSADIPFQLSKYVDNKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWIFVTITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNIGIRGYMYLKGPRGSVVTTNIYLNSTLYEGTKFIIKKYASGNEDNIVRNNDRVYINVVVKNKEYRLATNASQAGVEKILSALEIPDVGNLSQVVVMKSKDDQGIRNKCKMNLQDNNGNDIGFIGFHLYDNIAKLVASNWYNRQVGKASRTFGCSWEFIPVDDGWGESSL

SEQ ID NO: 9 - protein, 1292 aa, Clostridium botulinum
MPFVNKQFNYRDPVNGVDIAYIKIPNAGQMQPVKAFKIHEGVWVIPERDTFTNPEEGDLNPPPEAKQVPVSYYDSTYLSTDNEKDNYLKGVIKLFDRIYSTGLGRMLLSFIVKGIPFWGGSTIDTELKVIDTNCINVIEPGGSYRSEELNLVITGPSADIIQFECKSFGHDVFNLTRNGYGSTQYIRFSPDFTFGFEESLEVDTNPLLGAGTFATDPAVTLAHELIHAAHRLYGIAINPNRVLKVKTNAYYEMSGLEVSFEELRTFGGNDTNFIDSLWQKKFSRDAYDNLQNIARILNEAKTIVGTTTPLQYMKNIFIRKYFLSEDASGKISVNKAAFKEFYRVLTRGFTELEFVNPFKVINRKTYLNFDKAVFRINIVPDENYTINEGFNLEGANSNGQNTEINSRNFTRLKNFTGLFEFYKLLCVRGIIPFKTKSLDEGYNKALNDLCIKVNNWDLFFSPSEDNFTNDLDKVEEITADTNIEAAEENISSDLIQQYYLTFDFDNEPENISIENLSSDIIGQLEPMPNIERFPNGKKYELDKYTMFHYLRAQEFEHGDSRIILTNSAEEALLKPNVAYTFFSSKYVKKINKAVEAVIFLSWAEELVYDFTDETNEVTTMDKIADITIIVPYIGPALNIGNMVSKGEFVEAILFTGVVALLEFIPEYSLPVFGTFAIVSYIANKVLTVQTINNALSKRNEKWDEVYKYTVTNWLAKVNTQIDLIREKMKKALENQAEATRAIINYQYNQYTEEEKNNINFNIDDLSSKLNRSINRAMININKFLDQCSVSYLMNSMIPYAVKRLKDFDASVRDVLLKYIYDNRGTLILQVDRLKDEVNNTLSADIPFQLSKYVNDKKLLSTFTEYIKNIVNTSILSIVYKKDDLIDLSRYGAKINIGDRVYYDSIDKNQIKLINLESSTIEVILKNAIVYNSMYENFSTSFWIKIPKYFSKINLNNEYTIINCIENNSGWKVSLNYGEIIWTLQDNKQNIQRVVFKYSQMVNISDYINRWMFVTITNNRLTKSKIYINGRLIDQKPISNLGNIHASNKIMFKLDGCRDPRRYIMIKYFNLFDKELNEKEIKDLYDSQSNPGILKDFWGNYLQYDKPYYMLNLFDPNKYVDVNNIGIRGYMYLKGPRGSVMTTNIYLNSTLYMGTKFIIKKYASGNEDNIVRNNDRVYINVVVKNKEYRLATNASQAGVEKILSALEIPDVGNLSQVVVMKSKDDQGIRNKCKMNLQDNNGNDIGFVGFHLYDNIAKLVASNWYNRQVGKASRTFGCSWEFIPVDDGWGESSL

SEQ ID NO: 10 - protein, 1296 aa, Clostridium botulinum
MPFVNKQFNYNDPENGVDIAYIKIPNAGKMQPVKAFKIHNKVWVIPERDIFTNPEEVDLNPPPEAKQVPISYYDSAYLSTDNEKDNYLKGVIKLFERIYSTDLGRMLLISIVRGIPFWGGGKIDTELKVIDTNCINIIQLDDSYRSEELNLAIIGPSANIIESQCSSFRDDVLNLTRNGYGSTQYIRFSPDFTVGFEESLEVDTNPLLGAGKFAQDPAVALAHELIHAEHRLYGIAINTNRVFKVNTNAYYEMAGLEVSLEELITFGGNDAKFIDSLQKKEFSLYYYNKFKDIASTLNKAKSIVGTTASLQYMKNVFKEKYLLSEDATGKFLVDRLKFDELYKLLTEIYTEDNFVKFFKVLNRKTYLNFDKAVFKINIVPDVNYTIHDGFNLRNTNLAANFNGQNIEINNKNFDKLKNFTGLFEFYKLLCVRGIITSKTKSLDEGYNKALNELCIKVNNWDLFFSPSEDNFTNDLDKVEEITSDTNIEAAEENISLDLIQQYYLNFNFDNEPENTSIENLSSDIIGQLEPMPNIERFPNGKKYELNKYTMFHYLRAQEFKHSNSRIILTNSAKEALLKPNIVYTFFSSKYIKAINKAVEAVTFVNWIENLVYDFTDETNEVSTMDKIADITIVIPYIGPALNIGNMIYKGEFVEAIIFSGAVILLEIVPEIALPVLGTFALVSYVSNKVLTVQTIDNALSKRNEKWDEVYKYIVTNWLAIVNTQINLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLIGQVNRLKDKVNNTLSADIPFQLSKYVDNKKLLSTFTEYIKNITNASILSIVYKDDDLIDLSRYGAEIYNGDKVYYNSIDKNQIRLINLESSTIEVILKKAIVYNSMYENFSTSFWIRIPKYFNSISLNNEYTIINCMENNSGWKVSLNYGEIIWTLQDTQEIKQRVVFKYSQMINISDYINRWIFVTITNNRITKSKIYINGRLIDQKPISNLGNIHASNKIMFKLDGCRDPHRYIVIKYFNLFDKELSEKEIKDLYDNQSNSGILKDFWGDYLQYDKSYYMLNLYDPNKYVDVNNVGIRGYMYLKGPRDNVMTTNIYLNSSLYMGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWRERPL

SEQ ID NO: 11 - protein, 1296 aa, Clostridium botulinum
MLFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTELGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGEHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPEVNYTIYDGFNLRNTNLAANFNGQNTEINNMNFTKLKNFTGLFEFYKLLCVRGIITSKTKSLDEGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIVLTNSVNEALLNPSSVYTFFSSDYVRKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWGEVYKYIVTNWLAKVNTQIDLIRKKMKEALENQAEATKAIINYQYNQYTEEEKNNINFNIGDLSSKLNDSINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASEINIGSKVNFDPIDKNQIQLFNLESSKIEIILKNAIVYNSMYENFSTSFWIKIPKYFSKINLNNEYTIINCIENNSGWKVSLNYGEIIWTLQDNKQNIQRVVFKYSQMVAISDYINRWIFITITNNRLNNSKIYINGRLIDQKPISNLGNIHASNNIMFKLDGCRDPHRYIWIKYFNLFDKELNEKEIKDLYDNQSNSGILKDFWGNYLQYDKPYYMLNLYDPNKYVDVNNVGIRGYMYLKGPRGSIVTTNIYLNSSLYMGTKFIIKKYASGNKDNIVRNNDRVYINVVVKNKEYRLATNASQAGVEKILSVLEIPDVGNLSQVVVMKSKNDQGIRNKCKMNLQDNNGNDIGFIGFHQFNNIDKLVASNWYNRQIERSSRTFGCSWEFIPVDDGWGESPL

SEQ ID NO: 12 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFAKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLSAQEFEHGKSRIDLTNSVNEALLNPSHVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFAIVSYIANKVLTVQTINNALSKRNEKWDEVYKYTVTNWLAKVNTQIDLIREKMKKALENQAEATKAIINYQYNQYTEEEKNNINFNIDDLSSKLNESINSAMININKFLDQCSVSYLMNSMIPYAVKRLKDFDASVRDVLLKYIYDNRGTLIGQVDRLKDKVNNTLSTDIPFQLSKYVDNQRLLSTFTEYIKNIINTSILSLRYENNHLIDLSRYASKINIGSRVNFDPIDKNQIQLFNLESSKIEVILKNAIVYNSMYENFSTSFWIKIPKYFSEISLNNEYTIINCIENNSGWKVSLNYGEIIWTLQDNKQNIQRVVFKYSQMVAISDYINRWIFITITNNRLTKSKIYINGRLIDQKPISNLGNIHASNKIMFKLDGCRDPRRYIMIKYFNLFDKELNEKEIKDLYDSQSNSGILKDFWGNYLQYDKPYYMLNLFDPNKYVDVNNVGIRGYMYLKGSRSTLLTTNIYLNSGLYMGTKFIIKKYASGNKDNIVRNNDRVYINVVVNNKEYRLATNASQAGVEKILSALEIPDIGNLSQVVVMKSKNDQGIRNKCKMNLQDNNGNDIGFIGFHKFNDIYKLVASNWYNRQIEISSRTFGCSWEFIPVDDGWGEKPL

SEQ ID NO: 13 - protein, 1296 aa, Clostridium botulinum
MPFVNKQFNYKDPVNGVDIAYIKIPNAGQMQPVKAFKIHNKIWVIPERDIFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIINFECKSFGHDVLNLTRNGYGSTQYIRFSPDFTFGFEESLEVDTNPLLGAGKFAIDPAVTLAHELIHAGHRLYGIAINPNRVFKVNTNAYYEMSGLEVSFEELRTFGGHDAKFIDSLQENEFRLYYYNKFKEVASILNKAKSIIGTTASLQYMKNVFKEKYLLSEDTSGKFSVDKLRFDKLYKMLTEIYTEDNFVKFFKVLNRKTYLNFDKAVFKMNIVPEVNYTIYDGFNLRNTNLAANFNGQNTEINNMNFTKLKNFTGLFEFYKLLCVRGIITSKTKSLDEGYNKALNDLCIKVNNWDLFFSPSEDNFTNDLNKGEEITSDTNIEAAEENISSDLIQQYYLTFNFDNEPENISIENLSSDIIGQLELMPNIERFPNGKKYELDKYTMFHYLRAQEFEYGNSRIVLINSVNEALLNPSSVYTFFSSDYVKKANEATEAAMFLGWVEQLVYDFTDETSEVSTMDKIADITIIVPYIGPALNIGNMVYKKKFEEALIFSGAVILLEFVPEIVLPILGTFALVSYTSNKVLTVRTIDNALSKRNEKWEEVYKYIVTNWLAKVNTQINLIRKKMKEALENQAEATKAIINYQYNQYTEEEKNNINFNIGDLSSKLNDSINKAMININKFLDQCSVSYLMNSMIPQGVKQLKDFDTSLRDSLLKYIYDNRGTLIGQVDRLKDKVNNTLSTDIPFQLSKYADNQRLLSTFTEYIKNIINTSILNLRYESNHLIDLSRYASKINIGSRVNFDPIDKNQIQLFNLESSKIEVILKNAIVYNSMYENFSTSFWIKIPKYFSKINLNNEYTIINCIENNSGWKVSLNYGEIIWTLQDNEQNIQRVVFKYSQMVNISDYINRWIFVTITNNRLTKSKIYINGRLIDQKPISNLGNIHASNKIMFKLDGCRDPHRYILIKYFNLFDKELNEKEIKDLYDNQSNSGILKDFWGDYLQYDKPYYMLNLYDPNKYIDVNNIGIRGYMYLKGPRGSVTTTNIYLNSMLYMGTKFIIKKHASGNKDNIVRNNDRVYINVLVKNKEYRLATNASQAGGEKILSAVEIPDVGNLSQVVVMKSKNDQGIRNKCKMNLQDNNGNDIGFIGFHQFNNIAKLVASNWYNRQIGKTSVTLGCSWELIPVDYGWGESSL

SEQ ID NO: 14 - protein, 1297 aa, Clostridium botulinum
MPFVNKQFNYKDTVNGIDIAYIKIPNAGQMQPVKAFKIHNKIWVIPERDTFTNPKEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHDVLNLTRNGYGSTQYIRFSPDFTFGFEESLEVDTNPLLGAGKFATDPAVTLAHELIHAEHRLYGIAINPNRVFKVNTNAYYEMSGLEVSFEELRTFGGHNAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPDENYTIKDGFNLKNTNLAANFNGQNTEINSRNFTKLKNFTGLFEFYKLLCVRGIIPFKTKSLDEGYNKALNDLCIKVNNWDLFFSPSEDNFTNDLDKVEEITSDTNIEAAEENISLDLIQQYYLTFDFDNEPENISIENLSSDIIGQLEPMPNIERFPNGKKYELDKYTMFHYLRAQEFEHSKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLVRKKMKEALENQAEATKAIINYQYNQYTEEEKNNINFNIDDLSSKLNESINSAMTNINKFLDQCSVSYLMNSMIPYAVKRLKDFDASVREVLLKYIYDNRGTLILQVDRLKDKVNNTLSADIPFQLSKYVDNKKLLSTFTEYIKNITNTSILSIVVDKDGRLIDLSRYGAEIYNGDKVSYNSIDKNQIKLINLESSAIEVILKNAIVYNSMYENFSTSFWIKIPKYFSKINLNNEYTIINCIENNSGWKVSLNYGEIIWTLQDNQQNIQRVVFKYSQMVNISDYINRWIFVTITNNRLDKSKIYINGRLIDQKPISNLGNIHASNNIMFKLDGCRDPRRYIVIKYFNLFDKELNEKEIKDLYDNQSNSGILKDFWGDYLQYDKPYYMLNLYDPNKYVDVNNIGIRGYMYLKGPRGSVVTTNIYLNSTLYMGTKFIIKKYASGNKDNIVRNNDRVYINVVVKNKEYRLATNALQAGVEKILSALEIPDVGNLSQVVVMKSKNDQGIRNKCKMNLQDNNGNDIGLIGFHQFNNIAKLVASNWYNRQVGKASRTFGCSWEFIPVDDGWGESSQ

SEQ ID NO: 15 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 16 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNINIDFNDINSKLNEGINQAVDNINNFINECSVSYLMKKMIPLAVEKLLDFDNTLKKNLLNYIDENKLYLIGSAEYEKSKVDKHLKTIIPFDLSMYTNNTILIEIFNKYNSEILNNIILNLRYRDNNLIDLSGYGANVEVYDGVELNDKNQFKLTSSTNSEIRVTQNQNIIFNSMFLDFSVSFWIRIPKYKNDGIQNYIHNEYTIINCIKNNSGWKISIRGNRIIWTLTDINGKTKSVFFEYSIREDISDYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSSVGEILTRSKYNQNSNYINYRNLYIGEKFIIRRKSNSQSINDDIVRKEDYIYLDFFNSNREWRVYAYKDFKEEEKKLFLANIYDSNEFYKTIQIKEYDEQPTYSCQLLFKKDEESTDEIGLIGIHRFYESGIVLKDYKNYFCISKWYLKEVKRKPYNPNLGCNWQFIPKDEGWIE

SEQ ID NO: 17 - protein, 1291 aa, Clostridium botulinum
MPVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPRIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNINIDFNDINSKLNEGINQAIDNINNFINECSVSYLMKKMIPLAVEKLLDFDNTLKKNLLNYIDENKLYLIGSAEYEKSKVDKHLKTIIPFDLSMYTNNTILIEIFNKYNSEILNNIILNLRYRDNNLIDLSGYGAKVEVYNGVELNDKNQFKLTSSANSKIRVTQNQDIIFNSMFLDFSVSFWIRIPKYKNDGIQNYIHNEYTIINCIKNNSGWKISIRGNKIIWTLTDINGKTKSVFFEYSIRKDVSEYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSSVGEILTRSKYNQNSNYINYRNLYIGEKFIIRRKSNSQSINDDIVRKEDYIYLDFFNLNQEWRVYAYKDFKKKEEKLFLANIYDSNEFYNTIQIKEYDEQPTYSCQLLFKKDEESTDEIGLIGIHRFYESGIVFKDYKDYFCISKWYLKEVKRKPYNPNLGCNWQFIPKDEGWIE

SEQ ID NO: 18 - protein, 1291 aa, Clostridium botulinum
MPVTINNFNYNDPIDNDNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVEQKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDTIQAEELYTFGGQDPSIISPSTDKSIYDKVLQNFRGIVDRLNKVLVCISDPNININIYKNKFKDKYKFVEDSEGKYSIDVESFNKLYKSLMFGFTEINIAENYKIKTRASYFSDSLPPVKIKNLLDNEIYTIEEGFNISDKNMGKEYRGQNKAINKQAYEEISKEHLAVYKIQMCKSVKVPGICIDVDNENLFFIADKNSFSDDLSKNERVEYNTQNNYIGNDFPINELILDTDLISKIELPSENTESLTDFNVDVPVYEKQPAIKKVFTDENTIFQYLYSQTFPLNIRDISLTSSFDDALLVSSKVYSFFSMDYIKTANKVVEAGLFAGWVKQIVDDFVIEANKSSTMDKIADISLIVPYIGLALNVGDETAKGNFESAFEIAGSSILLEFIPELLIPVVGVFLLESYIDNKNKIIKTIDNALTKRVEKWIDMYGLIVAQWLSTVNTQFYTIKEGMYKALNYQAQALEEIIKYKYNIYSEEEKSNININFNDINSKLNDGINQAMDNINDFINECSVSYLMKKMIPLAVKKLLDFDNTLKKNLLNYIDENKLYLIGSVEDEKSKVDKYLKTIIPFDLSTYTNNEILIKIFNKYNSEILNNIILNLRYRDNNLIDLSGYGAKVEVYDGVKLNDKNQFKLTSSADSKIRVTQNQNIIFNSMFLDFSVSFWIRIPKYRNDDIQNYIHNEYTIINCMKNNSGWKISIRGNRIIWTLIDINGKTKSVFFEYNIREDISEYINRWFFVTITNNLDNAKIYINGTLESNMDIKDIGEVIVNGEITFKLDGDVDRTQFIWMKYFSIFNTQLNQSNIKEIYKIQSYSEYLKDFWGNPLMYNKEYYMFNAGNKNSYIKLVKDSSVGEILIRSKYNQNSNYINYRNLYIGEKFIIRRKSNSQSINDDIVRKEDYIHLDFVNSNEEWRVYAYKNFKEQEQKLFLSIIYDSNEFYKTIQIKEYDEQPTYSCQLLFKKDEESTDDIGLIGIHRFYESGVLRKKYKDYFCISKWYLKEVKRKPYKSNLGCNWQFIPKDEGWTE

SEQ ID NO: 19 - protein, 1291 aa, Clostridium botulinum
MPVTINNFNYNDPIDNNNIIMMEPPFARGMGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVNDLPIVPNEKKFFMQSTDAIQAEELYTFGGQDPSIISPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVKAPGICIDVDNEDLFFIADKNSFSDDLSKNERIAYNTQNNYIENDFSINELILDTDLISKIELPSENTESLTDFNVYVPVYKKQPAIKKIFTDENTIFQYLYSQTFPLDIRDISLTSSFDDALLFSNKVYSFFSMDYIKTANKVVEAGLFAGWVKQIVDDFVIEANKSSTMDKIADISLIVPYIGLALNVGNETAKGNFENAFEIAGASILLEFIPELLIPVVGAFLLESYIDNKNKIIETINSALTKRDEKWIDMYGLIVAQWLSTVNTQFYTIKEGMYKALNYQAQALEEIIKYKYNIYSEKERSNINIDFNDVNSKLNEGINQAIDNINNFINECSVSYLMKKMIPLAVEKLLDFDNTLRKNLLNYIDENKLYLIGSAEYEKSKVDKYLKTSIPFDLSTYTNNTILIEIFNKYNSDILNNIILNLRYRDNKLIDLSGYGAKVEVYDGVKLNDKNQFKLTSSANSKIRVIQNQNIIFNSMFLDFSVSFWIRIPKYKNDGIQNYIHNEYTIINCMKNNSGWKISIRGNMIIWTLIDINGKIKSVFFEYSIKEDISEYINRWFFVTITNNSDNAKIYINGKLESHIDIRDIREVIANDEIIFKLDGNIDRTQFIWMKYFSIFNTELSQSNIEEIYKIQSYSEYLKDFWGNPLMYNKEYYMFNAGNKNSYIKLKKDSSVGEILTRSKYNQNSKYINYRDLYIGEKFIIRRKSNSQSINDDIVRKEDYIYLDFFNLNQEWRVYMYKYFKKEEEKLFLAPISDSDEFYNTIQIKEYDEQPTYSCQLLFKKDEESTDEIGLIGIHRFYESGIVFKEYKDYFCISKWYLKEVKRKPYNSKLGCNWQFIPKDEGWTE

SEQ ID NO: 20 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKNMEKEYRGQNKAINKQAYEEISKEHLAVYKIQMCKSVRAPGICIDVDNEDLFFIADKNSFSDDLSKNERIEYDTQSNYIENRSSIDELILDTNLISKIELPSENTESLTDFNVDVPVYEKQPAIKKFFTDENTIFQYLYSQTFPLDIRDISLTSSFDDALLFSNKVYSFFSMDYIKTANKVVEAGLFAGWVKQIVDDFVIEANKSNTMDKLADISLIVPYIGLALNVGNETAKGNFENAFEIAGASILLEFIPELLIPVVGAFLLESYIDNKNKIIKTIDNALTKRDEKWRDMYGLIVAQWLSTVNTQFYTIKEGMYKALNYQAQALEEIIKYKYNIYSEKEKSNINIDFNDINSKLNEGINQAIDNINNFINECSVSYLMKKMIPLAVEKLLDFDNTLKKNLLNYIDENKLYLIGSAEYEKSKVDKHLKTIIPFDLSMYTNNTILIEIFKKYNSEILNNIILNLRYRDNNLIDLSGYGANVEVYDGVELNDKNQFKLTSSTNSEIRVTQNQNIIFNSMFLDFSVSFWIRIPKYKNDGIQNYIHNEYTIINCIKNNSGWKISIRGNRIIWTLTDINGKTKSVFFEYSIREDISDYINRWFFVTITNNSDNAKIYINGKLESNIDIKDIGEVIANGEIIFKLDGDIDRTQFIWMKYFSIFNTELSQSNIKEIYKIQSYSEYLKDFWGNPLMYNKEYYMFNAGNKNSYIKLKKDSPVGEILTRSKYNQNSNYINYRNLYIGEKFIIRRKSNSQSINDDIVRKEDYIYLDFFNLNQEWRVYALKNFKKKEEKLFLAPISDSDEFYNTIQIKEYDEQPTYSCQLLFKKDEESTDEIGLIGIHRFYESGIVFKDYKYYFCISKWYLKEVKRKPYNPNLGCNWQFIPKDEGWIE

SEQ ID NO: 21 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTKNIYIENYFSINELILDTDLISGIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIIDDFVIEANKSSTMDKIADISLIVPYIGLALNVGNETAKGNFENAFEIAGASILLEFIPELLIPVVGAFLLESYIDNKNKIIKTIDNALTKRVEKWIDMYGLIVAQWLSTVNTQFYTIKEGMYKALNYQAQALEEIIKYKYNIYSEKEKLNINIDFNDINSKLNEGINQAIDNINNFINECSVSYLMKKMIPLAIEKLLDFDNALKKNLLNYIDENKLYLIGSVEEEKSKVDKFFKTIIPFDLSMYTNNTILIEMVNKYNSEILNNIILNLRYRDNNLIDSSGYGAKVEVYNGVELNDKNQFKLTSSANSKIKVTQNQNITFNSMFLDFSVSFWIRIPKYKNDGIQNYIHNEYTIINCMKNNSGWKISIRGNRIIWTLTDINGKTKSVFFEYSIREDISDYINRWFFVTITNNLDNAKIYINGKLESNIDIRDIREVIVNGEIIFKLDGEIDRTQFIWMKYFSIFNTELSQSNVKEIYKIQSYSKYLKDFWGNPLMYNKEYYMFNAGNKNSYIKLVKDSSVGEILTRSKYNQNSNYINYRNLYIGEKFIIRRKSSSQSISDDIVRKEDYIYLDFFNSNREWRVYAYKNFKGQEEKLFLANIYDSNEFYKTIQIKEYDEQPTYSCQLLFKKDEESTDEIGLIGIHNFYESGILFKDYKDYFCISKWYLKEVKKKPYSSNLGCNWQFIPKDEGWTE

SEQ ID NO: 22 - protein, 1292 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGGEERKEGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNGKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDDEIYTIEEGFNISDKNMGKEYRGQNKAINKQAYEEISKEHLAVYKIQMCKSVRAPGICIDVDNEDLFFIADKNSFSDDLSKNERIEYNTQSNYIENDFSINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVDDFVIEANKSNTMDKIADISLIVPYIGLALNVGNETAKGNFENAFEIAGSSILLEFIPELLIPVVGAFLLESYIDNKNKIIKTIDNALTKRDEKWIDMYGLIVAQWLSTVNTQFYTIKEGMYKALNYQAQALEEIIKYKYNIYSEKEKSNISIDFNDINSKLNEGINQAIDNINDFINECSVSYLMKKMIPLAVEKLLDFDNTLKKNLLNYIDENKLYLIGSAEYEKSKVDKHLKTIMTFDLSMYTNNTILIKMVNKYNSEILNNIILNLRYRDNNLIDLSGYGANVEVYDGVELNDKNQFKLTSSTNSEIRVTQNQNIIVNSMFLDFSVSFWIRIPKYKNDGIQNYIHNEYTIINCMKNNSGWKISIRGNRIIWTLIDINGKIKSVFFEYSIRKDVSEYINRWFFVTITNNLDNAKIYINGKLESNMDIRDIREVIANGEIIFKLDGDIDRTQFIWMKYFSIFNTELSQSNIEEIYKIQSYSEYLKDFWGNPLMYNKEYYMFNAGSKNSYIKLKKDSSVGEILTRSKYNQNSQYINYRDLYIGEKFIIKRKSNSQSINDDIVRKEDYIYLDFFNLNQEWRVYAYKDFKGQKEQKLFLANIHDSNEFYKTIQIKEYDEQPTYSCQLLFKKDEESTDEIGLIGIHRFYESGFVFQEYKYYFCISKWYLKEVKKKPYNPDLGCNWQFIPKDEGWTE

SEQ ID NO: 23 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 24 - protein, 1280 aa, Clostridium botulinum
MPITINNFNYSDPVDNKNILYLDTHLNTLANEPEKAFRIIGNIWVIPDRFSRDSNPNLNKPPRVTSPKSGYYDPNYLSTDSEKDTFLKEIIKLFKRINSREIGEELIYRLATDIPFPGNNNTPINTFDFDVDFNSVDVKTRQGNNWVKTGSINPSVIITGPRENIIDPETSTFKLTNNTFAAQEGFGALSIISISPRFMLTYSNATNNVGEGRFSKSEFCMDPILILMHELNHTMHNLYGIAIPNDQRISSVTSNIFYSQYKVKLEYAEIYAFGGPTIDLIPKSGRKYFEEKALDYYRSIAKRLNSITTANPSSFNKYIGEYKQKLIRKYRFVVESSGEVAVDRNKFAELYKELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDIKTDIFLSKDINVETEVIDYPDNVSVDQVILSKNTSEHGQLDLLYPIIEGESQVLPGENQVFYDNRTQNVDYLNSYYYLESQKLSDNVEDFTFTTSIEEALDNSGKVYTYFPKLADKVNTGVQGGLFLMWANDVVEDFTTNILRKDTLDKISDVSAIIPYIGPALNISNSVRRENFTEAFAVTGVTILLEAFQEFTIPALGAFVIYSKVQERNEIIKTIDNCLEQRIKRWKDSYEWMIGTWLSRITTQFNNISYQMYDSLNYQADAIKDKIDLEYKKYSGSDKENIKSQVENLKNSLDIKISEAMNNINKFIRECSVTYLFKNMLPKVIDELNKFDLKTKTELINLIDSHNIILVGEVDRLKAKVNESFENTIPFNIFSYTNNSLLKDIINEYFNSINDSKILSLQNKKNALVDTSGYNAEVRLEGDVQVNTIYTNDFKLSSSGDKIIVNLNNNILYSAIYENSSVSFWIKISKDLTNSHNEYTIINSIKQNSGWKLCIRNGNIEWILQDINRKYKSLIFDYSESLSHTGYTNKWFFVTITNNIMGYMKLYINGELKQSERIEDLNEVKLDKTIVFGIDENIDENQMLWIRDFNIFSKELSNEDINIVYEGQILRNVIKDYWGNPLKFDTEYYIINDNYIDRYIAPKSNILVLVQYPDRSKLYTGNPITIKSVSDKNPYSRILNGDNIMFHMLYNSGKYMIIRDTDTIYAIEGRECSKNCVYALKLQSNLGNYGIGIFSIKNIVSQNKYCSQIFSSFMKNTMLLADIYKPWRFSFENAYTPVAVTNYETKLLSTSSFWKFISRDPGWVE

SEQ ID NO: 25 - protein, 1256 aa, Clostridium phage
  source 1..1256: note = Phage D-1873
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYET

SEQ ID NO: 26 - protein, 1285 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGSDVEIIPQIERLQLREKALGHYKDIAKRLNNINKTIPSSWSSNIDKYKKIFSEKYNFDKDNTGNFVVNIDKFNSLYSDLTNVMSEVVYSSQYNVKNRTHYFSKHYLPVFANILDDNIYTIINGFNLTTKGFNIENSGQNIERNPALQKLSSESVVDLFTKVCLRLTRNSRDDSTCIQVKNNTLPYVADKDSISQEIFESQIITDETNVENYSDNFSLDESILDAKVPTNPEAVDPLLPNVNMEPLNVPGEEEVFYDDITKDVDYLNSYYYLEAQKLSNNVENITLTTSVEEALGYSNKIYTFLPSLAEKVNKGVQAGLFLNWANEVVEDFTTNIMKKDTLDKISDVSAIIPYIGPALNIGNSALRGNFKQAFATAGVAFLLEGFPEFTIPALGVFTFYSSIQEREKIIKTIENCLEQRVKRWKDSYQWMVSNWLSRITTQFNHISYQMYDSLSYQADAIKAKIDLEYKKYSGSDKENIKSQVENLKNSLDVKISEAMNNINKFIRECSVTYLFKNMLPKVIDELNKFDLKTKTELINLIDSHNIILVGEVDRLKAKVNESFENTIPFNIFSYTNNSLLKDMINEYFNSINDSKILSLQNKKNTLMDTSGYNAEVRVEGNVQLNPIFPFDFKLGSSGDDRGKVIVTQNENIVYNAMYESFSISFWIRINKWVSNLPGYTIIDSVKNNSGWSIGIISNFLVFTLKQNENSEQDINFSYDISKNAAGYNKWFFVTITTNMMGNMMIYINGKLIDTIKVKELTGINFSKTITFQMNKIPNTGLITSDSDNINMWIRDFYIFAKELDDKDINILFNSLQYTNVVKDYWGNDLRYDKEYYMINVNYMNRYMSKKGNGIVFNTRKNNNDFNEGYKIIIKRIRGNTNDTRVRGENVLYFNTTIDNKQYSLGMYKPSRNLGTDLVPLGALDQPMDEIRKYGSFIIQPCNTFDYYASQLFLSSNATTNRLGILSIGSYSFKLGDDYWFNHEYLIPVIKIEHYASLLESTSTHWVFVPASE

SEQ ID NO: 27 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 28 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGIQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYNNEPNANILKDFWGNYLLYDKEYYLLNVLKPNNFIDRRTDSTLSINNIRSTILLANRLYSGIKVKIQRVNNSSTNDNLVRKNDQVYINFVASKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 29 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHELIHSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 30 - protein, 1252 aa, Clostridium butyricum
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVGWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKAIIESKYNSYTLEEKNELTNKYDIEQIENELNQKVSIAMNNIDRFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFINRRTDSTLSINNIRSTILLANRLYSGIKVKIQRVNNSSTNDNLVRKNDQVYINFVASKTHLLPLYADTATTNKEKTIKISSSGNRFNQVVVMNSVGNNCTMNFKNNNGNNIGLLGFKADTVVASTWYYTHMRDNTNSNGFFWNFISEEHGWQEK

SEQ ID NO: 31 - protein, 1251 aa, Clostridium butyricum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFVPELLIPTILVFTIKSFLGSSDNKNKVIKAINNALKERDEKWKEVYSFIVSNWMTKINTQFNKRKEQMYQALQNQVNALKTIIEFKYNSYTLEEKKELKNNYDIEQIENELNQKVSIAMNNIDRFLTESSISYLMKLINEVKINKLREYDENVKTYLLDYIIQHGSILGESQQELNSMVIDTLNNSIPFKLSSYTDDKILISYFNKFFKRIKSSSVLNMRYKNDKYVDTSGYDSNININGEIFIYPTNKNQFTIFNSKPSEVNISQNDYIIYDNKYKNFSISFWVRIPNYDNKIVNINNEYTIINCMRDNNSGWKVSLNHNEIIWTLQDNARINQKLVFKYGNANGISDYINKWIFVTITNDRLGDSKLYINGHLIDQKSILNLGNIHVSDNILFKIVNCSYTRYIGIRYFNIFDKELDETEIQTLYSNEPNTNILKDFWGNYLLYDKGYYLLNVLKPNNFIDRRKDSTLSINNIRSTILLANRLYSGIKVKIQRVNDSSTNDRFVRKNDQVYINYISNSSSYSLYADTNTTDKEKTIKSSSSGNRFNQVVVMNSVGNNCTMNFKNNNGNNIGLLGFKADTVVASTWYYTHMRDHTNSNGCFWNFISEEHGWQEK

SEQ ID NO: 32 - protein, 1252 aa, Clostridium botulinum
MPTINSFNYNDPVNDRTILYIKPGGCQEFYKSFNIMKNIWIIPERNVIGTTPQDFHPPTSLKNGDSSYYDPNYLQSYEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFKDNSMNEFIQDPALTLMHELIHSLHGLYGAKGITTQYTITQQQNPLITNIKGTNIEEFLTFGGTDLNIITSAQYNDIYTNLLADYKKIASKLSKVQVSNPLLNPYKDVFEKKYGLDKDASGIYSVNINKFNDIFKKLYSFTEFDLATKFQVKCRQTYIGQYKYFKLSNLLNNSIYNISEGYNINTLKVNFRGQNTNLNPRIITPLTGRGLVKKIIRFCKNIVFSKGIRKSICIEINNGELFFVASDNSYNDDNINTPKEIDDTVTSNNNYENDLDQVILNFNSESAPGLSDEKLNLTIQNDAYIPKYDSNGTSDIEQHDVNELNVFFYLDAQKVPEGENNIDFTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNNKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILNDFWGNYLLYDKEYYLLNVLKPNNFINRRTDSTLSINNIRSTILLANRLYSGIKVKIQRVNNSSTNDNLVRKNDQVYINFVDSKTHLLPLYADTATTNKEKTIKISSSGNRFNQVVVMNSVGNNCTMNFKNNNGNNIGLLGFKADTVVASTWYYTHMRDNTNSNGFFWNFISEEHGWQEK

SEQ ID NO: 33 - protein, 1252 aa, Clostridium botulinum
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGRILLEELSKANPYLGNDNTPDNQFHIGDASAVEIKFSNGNQSILLPNVIIMGAEPDLFETNSSNISLRNNYMPSNHGFGSIAIVTFSPEYSFRFNDNSMNEFIQDPALTLMHELIHSLHGLYGAKRITTKYTITQQQNPLITNIRGTNIEEFLTFGGTDLNIITSAQYNDIYTNLLADYKKIASKLSKVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 34 - protein, 1252 aa, Clostridium botulinum
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGRILLEELSKANPYLGNDNTPDNQFHIGDASAVEIKFSNGNQSILLPNVIIMGAEPDLFETNSSNISLRNNYMPSNHGFGSIAIVTFSPEYSFRFNDNSMNEFIQDPALTLMHELIHSLHGLYGAKRITTKYTITQQQNPLITNIRGTNIEEFLTFGGTDLNIITSAQYNDIYTNLLADYKKIASKLSKVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSYIEQHDVNELNVFFYLDAQKVPEGENNVNLTSSIDTALLEQPKIYTFFSSEFINNVNKTVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFIDRRTDSTLSINNIRSTILLANRLYSGIKVKIQRVNNSSTNDNLVRKNDQVYINFVASKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 35 - protein, 1251 aa, Clostridium botulinum
MPKINSFNYNDPVNDNTILYIKPGGCQQFYKSFNIMKNIWIIPERNVIGTIPQNFLPPTSLKNGDSSYYDPNYLQNDQEKDRFLKIVTKVFNRINDNLSGRILLEELSKANPYLGNDNTRDDDFIINDGSAVPIQFSNGSQSILLPTVIIMGAEPDLFETNSSNVSLINNYSPSNHGFGSIAIVTFSPEYSFRFNDNSMNEFIQDPALTLIHELIHSLHGLYGAKGITTKYTITQQQNPLITNIRGINIEEFLTFGGNNLNIITSSQLNDIYTNLLDDYKKIASKLSKVQVSNPQLNPYKDVFQEKYGLDKNASGIYSVNINKFNDIFKKLYSFTEFDLATKFQVKCRETYIGQYKYFKLSNLLNDSIYNISEGYNINTLNVNFRGQNPNLNPRIITPITDRGLVKKIIRFCKNIVSVKGIRKSICIEVNNGDLFFVASEKSYNNDSINIPKEIDDTVTLNNNYENDLDQVILNFNSESAPGLSDKKLNISIQDDVYIPKYDSNGTSDIEQYDVSELNVFFYLDAQKVPEGENNVNLTSSIDTALLEQSKIYTFFSSEFINNVNKPVQAALFVGWIQQVLVDFTTEATQKSTVDKIADISIVVPYIGLALNIGNESQKGNFKDALELLGAGILLEFVPELLIPTILVFTIKSFLGSSDNKNKVIKAINNALKERDEKWKEVYSFIVSNWITKINTQFNKRKEQMYQALQNQVNALKTIIESKYNSYTLEEKNELTNKYDIEQIENELNQKVSIAMNNIDRFLTESSISYLMKLINEVKINKLREYDENVKTYLLDYITKHGSILGESQQELNSMIIDTLNNSIPFKLSSYTDDKILISYFNKFFKTIKSSSVLSMRYKNDKYIDTSGYDSNININGDVFIYPTNKNQFGIYNSKLSEVNISQNDYIIYDNKYKNFSISFWVRIPNYNNKIVNVNNEYTIINCMRDNNSGWKISLNHNEIIWTLQDNAGINQKLVFKYGNANGISDYINKWIFVTITNDRLGYSKLYINGHLIDQKSILNLGNIHVSDNILFKIVNCSYTRYIGMRYFNIFDKELDETEIQTLYNNEPNANVLKDFWGNYLLYNKEYYLLNMLKPSKTISHNRDLTFSIYNNRNIVNGLYRLYSGIKVKIQKINDSDTRDNIVRDNDQVYVNYINGNVYYSLYADTNATNKEKTIKSSTSGNRFNQVVVMNSVRNNCTMNFKNNNGHDIGLLGFKSNALVASTWYYTNMRDHTNSNGCFWSFIPEENGWQEH

SEQ ID NO: 36 - protein, 1252 aa, Clostridium botulinum
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGGILLEELSKANPYLGNDNTPNNQFHIGDASAVEIKFSNGSQSILLPTVIIMGAEPDLFETNSSNISLKNNYMPSNHGFGSIAIVTFSPEYSFRFNDNSMNEFIQDPALTLMHELIHSLHGLYGAKGITTKYTITQQQNPLITNIRGTNIEEFLTFGGTDLNIITNAQSNDIYTNLLADYKKIASKLSQVQVSNPQLNPYKDIFQEKYGLDKNASGIYSVNINKFDDIFKKLYSFTEFDLATKFQVKCRQTYIGQYKYFKLSNLLNNSIYNISEGYNINTLKVNFRGQNTNLNPRIITQLTGRGLVKKIIRFCKNIVFSKGITKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKTIIESKYNSYTLEEKNELTNKYNIEQIENELNQKVSIAMNNIEIFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDKKSILNLGNIHVSDNILFKIVNCSYTRYIGMRYFNIFDKELDKTEIETLYNNEPNTNILKDFWGNYLLYDKEYYLLNVLKPNNVIDSNRDSTFSIHNIRSTIVLANKLYLGIKVKIQRVNNSSTNDNLVRKNDQVYINFVPIKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 37 - protein, 1252 aa, Clostridium botulinum
MPKINSFNYNDPVNDKTILYIKPGGCQQFYKSFNIMKNIWIIPERNVIGTIPQDFLPPTSLKNGDSSYYDPNYLQSNEEKDRFLKIVTKIFNRINDNLSGGILLEELSKANPYLGNDNTPNNQFHIGDASAVEIKFSNGSQSILLPTVIIMGAEPDLFETNSSNISLKNNYMPSNHGFGSIAIVTFSPEYSFRFNDNSMNEFIQDPALTLMHELIHSLHGLYGAKGITTKYTITQQQNPLITNIRGTNIEEFLTFGGTDLNIITNAQSNDIYTNLLDDYKKIASKLSQVQVSNPQLNPYKDVFQEKYGLDKDANGIYSVNINKFNDIFKKLYSFTEFDLATKFQVKCRKTYIGHHKYFRLSDLLNDSIYNISDGYNINTLKVNFRGQNTNLNTRIITPITGRGVVRKIIRFCTNIFSPKGIRKSICIEVNNGELFFVASENSYNDDNINTSKEIDDTVTSNNNYENDLDQVILNFNSESAPGLSDEKLNLTIQDDAYIPKYDSNGTSDIEQYDVSELNVFFYLDAQKVPEGENNVDFTSSIDTALLEQPKIYTFFSSKFISNLNKTMQAALFVSWIQQVLVDFTTEATQKSTVDKIADISIVVPYIGLALNIGNEAQKGNFKDALELLGAGILLEFEPELLIPIILVFTIKSFLGSSDNKNKVIKAINNALKERDENWKEVYSFIVSNWMTKINTQFNKRKEQMYQALQNQVNAIKTIIESKYNSYTLEEKNELKNKYDIEQIENELNQTVSIAMNNIEIFLTESSISYLMKLINEVKINKLKEYDENVKTYLLDYIIKHGSILGESQQELNSMVIDTLNNSIPFKLSSYTDDKILISYFNKFFKTIKSSSVLNMRYKNDKYIDTSGYDSNINIKGDVFIYPTNKNQFGIYNNKLSEVNISQNDYIIYDNKYKNFSISFWVRIPNYDNKIVNVNNEYTIINCMRDNNSGWKVSLNHNEIIWTLQDNAGINQKLVFKYGNANGISDYINKWIFVTITNDRLGDSKLYINGNLIDKKSILNLGNIHVSDNILFKIVNCSYTRYIGMRYFNIFDKELDKTEIETLYNNEPNTNILKDFWGNYLLYDKEYYLLNVLKPNNVIDSNRDSTFSIHNIRSTIVLANRLYSGIKVKIQRVNNSSTNDNLVRKNDQVYINFVASKTHLFPLYADTNTTNKEKTIKSSSSGNRFNQVVVMNSVGNNCTMNFKNNNGNNIGMLGFKDNTLVASTWYYTHMRDNTNSNGCFWNFISEEHGWQEK

SEQ ID NO: 38 - protein, 1251 aa, Clostridium botulinum
MPKINSFNYNDPVNDRTILYIKPGGCQQFYKSFNIMKNIWIIPERNVIGTIPQDFQPPTSLKNGDSSYYDPNYLQSNEEKDRFLKIVTKIFNRINDNLSGGILLEELSKANPYLGNDNTPDGDFIINDASAVPIQFSNGSQSILLPNVIIMGAEPDLFETNSSNISLINNYRPSNHGFGSIAIVTFSPEYSFRFNDNSMNEFIQDPALTLMHELIHSLHGLYGAKGITTKYTITQQQNSLITNIRGINIEEFLTFGGNDLNIITSSQFNDIYTNLLDDYKKIASKLSQVRVSNPQLNPYKDVFQEKYGLDKDASGIYSVNINKFNDIFKKLYSFTEFDLATKFQVKCRETYIGQYKYFQLSNLLNDSIYNISEGYNINNLKVNFRGQNANLNPRIITPITGRGLVKKIIRFCKNIVSVKGIRKSICIEVNNGELFFVASENSYNDDNINTPKEIDDTVTSNNNYENDLDQVILNFNSESAPGLSDEKLNLTIQDDAYIPKYDSNGTSDIEQHDVNELNVFFYLDAQKVPEGENNVNLTSSIDTALLEQPKIYTFFSSEFINNVNKPVQAVLFVSWIQQVLVDFTTEATQKSTVDKIADISIVVPYIGLALNIGNEAQKGNFKDALELLGAGILLEFVPELLIPTILVFTIKSFLGSSDNKNKIIKAINNALKERDEKWKEVYSFIVSNWITKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSTLGESQQELNSMVINTLNNSIPFKLSSYTDDKILISYFNKFFKRIKSSSVLNMRYKNDKYVDTSGYDSNININGEIFIYPTNKNQFSIFNSKPSEVNISQNDYIIYDNKYKNFSISFWVRIPNYDNKIVNVNNEYTIINCMRDNNSGWKVSLNHNEIIWTLQDNAGINQKLAFNYGNSNGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNSIISHRRDLTFSFYNHRYIVNGLYRLYSGIKVKIQRVNDSSTNDQFVRKNDQVYINYIYNNLSYSLYADTNIKDKEKTIKSSLSGNIFNQVVVMNSVGNNCTMNFKNNNGNNIGLLGFKDNTLVASTWYYTHMRDNTNSNGCFWNFISEEHGWQEK

SEQ ID NO: 39 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 40 - protein, 1280 aa, Clostridium botulinum
MPVVINSFNYNDPVNDETILYMQKPYEERSRKYYKAFEIMPNVWIMPERDTIGTKPDEFQVPDSLKNGSSAYYDPNYLTTDAEKDRYLKTMIKLFNRINSNPTGKVLLEEVSNARPYLGDDDTLINEFLPVNVTTSVNIKFSTDVESSIISNLLVLGAGPDIFKAYCTPLVRFNKSDKLIEPSNHGFGSINILTFSPEYEHIFNDISGGNHNSTESFIADPAISLAHELIHALHGLYGAKAVTHKESLVAERGPLMIAEKPIRLEEFLTFGGEDLNIIPSAMKEKIYNDLLANYEKIATRLREVNTAPPGYDINEYKDYFQWKYGLDRNADGSYTVNRNKFNEIYKKLYSFTEIDLANKFKVKCRNTYFIKYGFVKVPNLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEESKVYTFFSSEFIDTINKPVNAALFIDWISKVIRDFTTEATQKSTVDKIADISLIVPYVGLALNIVIEAEKGNFEEAFELLGAGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESKYNINNIEEELNKKVSLAMKNIERFMTESSISYLMKLINEAEVGKLKEYDKHVKSDLLDYILYHKLILGEQTKELIDLVTSTLNSSIPFELSSYTNDKILIIYFNRLYKKIKDSSILDMRYENNKFIDISGYGSNISINGNVYIYSTNRNQFGIYSGRLSEVNIAQNNDIIYNSRYQNFSISFWVTIPKHYRPMNRNREYTIINCMGNNNSGWKISLRTIRDCEIIWTLQDTSGNKEKLIFRYEELASISDYINKWIFVTITNNRLGNSRIYINGNLIVEKSISNLGDIHVSDNILFKIVGCDDETYVGIRYFKVFNTELDKTEIETLYSNEPDPSILKDYWGNYLLYNKKYYLFNLLRKDKYITRNSGILNINQQRGVTGGISVFLNYKLYEGVEVIIRKNAPIDISNTDNFVRKNDLAYINVVDHGVEYRLYADISITKSEKIIKLIRTSNPNDSLGQIIVMDSIGNNCTMNFQNNDGSNIGLLGFHSDDLVASSWYYNHIRRNTSSNGCFWSFISKEHGWKE

SEQ ID NO: 41 - protein, 1279 aa, Clostridium botulinum
MPVVINSFNYNDPVNDETILYMQKPYEERSRKYYKAFEIMPNVWIMPERDTIGTKPDDFQVPDSLKNGSSAYYDPNYLTTDAEKDRYLKTMIKLFNRINSNPTGKVLLEEVSNARPYLGDDDTLINEFFPVNVTTSVNIKFSTDVESSIISNLLVLGAGPDIFKAYCTPLVRFNKSDKLIEPSNHGFGSINILTFSPEYEHIFNDISGGDHNSTESFIADPAISLAHELIHALHGLYGAKAVTHKETIEVKRGPLMIAEKPIRLEEFLTFGGEDLNIIPSAMKEKIYNDLLANYEKIATRLREVNTAPPEYDINEYKDYFQWKYGLDRNADGSYTVNRNKFNGIYKKLYSFTEIDLANKFKVKCRNTYFIKYGFVKVPDLLDDDIYTVSEGFNIGNLAVNNRGQNINLNPKIIDSIPDKGLVEKIIKFCKSIIPRKGTKQSPSLCIRVNNRELFFVASESSYNESDINTPKEIDDTTNLNNNYRNNLDEVILDYNSETIPQISNRTLNTLVQDNSYVPRYDSNGTSEIEEYDVVDFNVFFYLHAQKVPEGETNISLTSSIDTALLEKSKVYTFFSSEFIDTINESVNAALFIDWINKVIRDFTTEATQKSTVDKIADISLIVPYVGLALNIVIDAEKGNFQEAFELLGAGILLEFVPELTIPVILVFTIKSYIDSYENKNKAIKAINNALIEREAKWKEIYSWIVSNWLTKINTQFNKRKEQMYQALQNQVDAIKTAIEYKYNNYTSDEKNRLESEYNINNIEEELNKKVSLAMKNIERFMTESSISYLMKLINEAEVGKLKKYDRHVKSDLLDYILYHKLILGDQTKELIDLVTSTLNSSIPFELSSYTNDKILIIYFNRLYKKIKDSSILDMRYENNKFIDISGYGSNISINGNVYIYSTNRNQFGIYSDRLSEVNIAQNNDIIYNSRYQNFSISFWVRIPKHYGPMNRNREYTIINCMGNNNSGWKISLRNIRDCEIIWTLQDTSGNKEKLIFRYEELANISDYINKWIFVTITNNRLGNSRIYINGNLIVEKSISNLGDIHVSDNILFKIVGCDDKTYVGIRYFKVFNTELDKTEIETLYSNEPDPSILKDYWGNYLLYNKKYYLFNLLRKDKYITRNSGILNINQQRGVTEGSVFLNYKLYEGVEVIIRKNGPIDISNTDNFVRKNDLAYINVVYHDVEYRLYADISITKPEKIIKLIRTSNPNDSLGQIIVMDSIGNNCTMNFQNNNGGNIGLLGFHSDNLVASSWYYNNIRRNTSSNGCFWSFISKEHGWQE

SEQ ID NO: 42 - protein, 1277 aa, Clostridium botulinum
MPVVINSFNYDDPVNDDTILYMQIPYEEKSKKYYKAFEIMRNVWIMPERNTIGTNPSDFDPPASLKNGSSAYYDPNYLTTDAEKDRYLKTTIKLFKRINSNPAGEVLLQEISYAKPYLGNDHTPINEFHPVTRTTSVNIKSSTNVESSIILNLLVLGAGPNIFENSSYPVRKLMNSGEVYDPSNDGFGSINIVTFSPEYEYTFNDISGGHNSSTESFIADPAISLAHELIHALHGLYGARGVTYKETIKVKQAPLMIAEKPIRLEEFLTFGGQDLNIITSAMKEKIYNDLLANYEKIATRLSEVNSAPPEYDINEYKNYFQWKYGLDKNADGSYTVNENKFNEIYKKLYSFTEIDLANKFKVKCRNTYFIKYGFLKVPNLLDDDIYTVSEGFNIGNLAVNNRGQNINLNPKIIDSIPDKGLVEKIVKLCKSIIPRKGTKAPPRLCIRVNNRELFFVASESSYNENDINTPKEIDDTTNLNNNYRNNLDEVILDYNSETIPQISSQTLNTLVQDDSYVPRYDSNGTSEIEEHNVVDLNAFFYLHAQKVPEGETNISLTSSIDTALSEESKVYTFFSSEFINNINKPVHAALFIGWISQVIRDFTTESTQKSTVDKIADISLIVPYVGLALNIGNDARKGNFKEAFELLGAAILLEVVPELLIPVILVFTIKSFIDSSKNEDKIIKAINNSLIEREAKWKEVYSWIVSNWLTRINTQFNKRKEQMYQALQNQVDAIKTVIEYKYNSYTSDEKNRLESEYNINNIEEELNKKVSLAMKNIERFIAESSISYLMKLINEAKVSELREYDEGVKEYLLDYILKNGSILGDHVQELNDLVTSTLNSSIPFELSSYTNDKILIIYFNKLYKKIKDNCILDMRYENNKFIDISGYGSNISINGELYIYTTNRNQFTIYSGKLSEVNIAQNNDIIYNSRYQNFSISFWVRIPRYSNIVNLNNEYTIINCMGNNNSGWKISLNYNKIIWTLQDTAGNNEKLVFNYTQMISISDYINKWIFVTITNNRLGNSRIYINGNLIDQKSISNLGDIHVSDNILFKIVGCNDTRYVGIRYFKVFDTELDKTEIETLYSDEPDPSILKDFWGNYLLYNKRYYLLNLLRKDNAITQSSTFLSISRARGVDRKANIFSNKRLYKGVEVIIRKNEPIDISNTDNFVRKGDLAYINVVDRDVEYRLYANTSNAQPEKTIKLIRTSNSNDSLDQIIVMDSIGNNCTMNFQNNNGGNIGLLGFHSNTLVASSWYYNNIRRNTSSNGCFWSFISKEHGWQE

SEQ ID NO: 43 - protein, 1277 aa, Clostridium botulinum
MPVEINSFNYDDLVNDNTILYIRPPYYERSNTYFKAFNIMENVWIIPERYRLGIEASKFDPPDSLKAGSDGYFDPNYLSTNTEKNRYLQIMIKLFKRINSNEAGKILLNQIKDAIPYLGNSYTAEDQFTTNNRTISFNVRLANGTIEQEMANLIIWGPGPDLTTNRTGGTTYTPAQSLEAIPYKEGFGSIMTIEFSPEYATAFNDISLTSHAPSLFIKDPALILMHELIHVLHGLYGTYTTGFKIKPNITEPYMEVTKPITSGEFLTFGGNDVNKIPQLIQSQLRSKVLDDYEKIASRLNKVNRATAEINIDKFKYSYQLKYQFVKDSNGVYSVDLDKFNKLYDKIYSFTEFNLAHEFKIKTRNSYLAKNFGPFYLPNLLDNSIYNEADGFNIGDLSVNYKGQVIGSDIDSIKKLEGQGVVSRVVRLCLNSSFKKNTKKPLCITVNNGDLFFIASEDSYGEDTINTPKEIDDTTTLVPSFKNILDKVILDFNKQVTPQIPNRRIRTDIQEDNYIPEYDSNGTSEIEEYNVVDLNAFFYLHAQKVPEGETNISLTSSIDTALSEESKVYTFFSSEFIDTINEPVNAALFIDWISKVIRDFTTEATQKSTVDKIADISLIVPYVGLALNIVNETEKGNFKEAFELLGAGILLEFVPELAIPVILVFTIKSYIDSYENKNKIIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFITESSISYLMKLINEAEVGKLKEYDKRVKRHLLEYIFDYRLILGEQGGELIDLVTSTLNTSIPFELSSYTNDKILIIYFNRLYKKIKDSSILDMRYENNKFIDISGYGSNISINGNVYIYSTNRNQFGIYDDRLSEVNIAQNNDIIYNSRYQNFSISFWVRIPKHYRPMNHNREYTIINCMGNNNSGWKISLRTTGDCEIIWTLQDTSGNKKKLIFRYSQLGGISDYINKWIFVTITNNRLGNSRIYINGNLIVEKSISNLGDIHVSDNILFKIVGCDDKMYVGIRYFKVFNTELDKTEIEILYSNEPDPSILKDYWGNYLLYNKKYYLLNLLRNDKYITRNSDILNISHQRGVTKDLFIFSNYKLYEGVEVIIRKNGPIDISNTDNFVRKNDLAYINVVDHGVEYRLYADISITKPEKIIKLIRRSNPDDSLGQIIVMDSIGNNCTMNFQNNNGGNIGLLGFHSDNLVASSWYYNNIRRNTSSNGCFWSFISKEHGWQE

SEQ ID NO: 44 - protein, 1274 aa, Clostridium botulinum
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKGTKAPPRLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELSSYTNDKILIIYFNRLYKKIKDSSILDMRYENNKFIDISGYGSNISINGNVYIYSTNRNQFGIYNSRLSEVNIAQNNDIIYNSRYQNFSISFWVRIPKHYKPMNHNREYTIINCMGNNNSGWKISLRTVRDCEIIWTLQDTSGNKENLIFRYEELNRISNYINKWIFVTITNNRLGNSRIYINGNLIVEKSISNLGDIHVSDNILFKIVGCDDETYVGIRYFKVFNTELDKTEIETLYSNEPDPSILKNYWGNYLLYNKKYYLFNLLRKDKYITLNSGILNINQQRGVTEGSVFLNYKLYEGVEVIIRKNGPIDISNTDNFVRKNDLAYINVVDRGVEYRLYADTKSEKEKIIRTSNLNDSLGQIIVMDSIGNNCTMNFQNNNGSNIGLLGFHSNNLVASSWYYNNIRRNTSSNGCFWSSISKENGWKE

SEQ ID NO: 45 - protein, 1268 aa, Clostridium baratii
MPVNINNFNYNDPINNTTILYMKMPYYEDSNKYYKAFEIMDNVWIIPERNIIGKKPSDFYPPISLDSGSSAYYDPNYLTTDAEKDRFLKTVIKLFNRINSNPAGQVLLEEIKNGKPYLGNDHTAVNEFCANNRSTSVEIKESKGTTDSMLLNLVILGPGPNILECSTFPVRIFPNNIAYDPSEKGFGSIQLMSFSTEYEYAFNDNTDLFIADPAISLAHELIHVLHGLYGAKGVTNKKVIEVDQGALMAAEKDIKIEEFITFGGQDLNIITNSTNQKIYDNLLSNYTAIASRLSQVNINNSALNTTYYKNFFQWKYGLDQDSNGNYTVNISKFNAIYKKLFSFTECDLAQKFQVKNRSNYLFHFKPFRLLDLLDDNIYSISEGFNIGSLRVNNNGQNINLNSRIVGPIPDNGLVERFVGLCKSIVSKKGTKNSLCIKVNNRDLFFVASESSYNENGINSPKEIDDTTITNNNYKKNLDEVILDYNSDAIPNLSSRLLNTTAQNDSYVPKYDSNGTSEIKEYTVDKLNVFFYLYAQKAPEGESAISLTSSVNTALLDASKVYTFFSSDFINTVNKPVQAALFISWIQQVINDFTTEATQKSTIDKIADISLVVPYVGLALNIGNEVQKGNFKEAIELLGAGILLEFVPELLIPTILVFTIKSFINSDDSKNKIIKAINNALRERELKWKEVYSWIVSNWLTRINTQFNKRKEQMYQALQNQVDGIKKIIEYKYNNYTLDEKNRLKAEYNIYSIKEELNKKVSLAMQNIDRFLTESSISYLMKLINEAKINKLSEYDKRVNQYLLNYILENSSTLGTSSVQELNNLVSNTLNNSIPFELSEYTNDKILISYFNRFYKRIIDSSILNMKYENNRFIDSSGYGSNISINGDIYIYSTNRNQFGIYSSRLSEVNITQNNTIIYNSRYQNFSVSFWVRIPKYNNLKNLNNEYTIINCMRNNNSGWKISLNYNNIIWTLQDTTGNNQKLVFNYTQMIDISDYINKWTFVTITNNRLGHSKLYINGNLTDQKSILNLGNIHVDDNILFKIVGCNDTRYVGIRYFKIFNMELDKTEIETLYHSEPDSTILKDFWGNYLLYNKKYYLLNLLKPNMSVTKNSDILNINRQRGIYSKTNIFSNARLYTGVEVIIRKVGSTDTSNTDNFVRKNDTVYINVVDGNSEYQLYADVSTSAVEKTIKLRRISNSNYNSNQMIIMDSIGDNCTMNFKTNNGNDIGLLGFHLNNLVASSWYYKNIRNNTRNNGCFWSFISKEHGWQE

SEQ ID NO: 46 - protein, 1281 aa, Clostridium botulinum
MPVVINSFNYNDPVNDDTILYMQIPYEEKSKKYYKAFEIMRNVWIIPERNTIGTDPSDFDPPASLKNGSSAYYDPNYLTTDAEKDKYLKTTIKLFKRINSNPAGEVLLQEISYAKPYLGNEHTPINEFHPVTRTTSVNIKSSTNVKSSIILNLLVLGAGPNIFENSCYPVRKLMDSGEVYDPSNDGFGSINIVTFSPEYEYTFNDISGGHNSSTESFIADPAISLAHELIHALHGLYGARGVTYKETIKVKQAPLMIAEKPIRLEEFLTFGGQDLNIITSAMKEKIYNNLLANYEKIATRLSEVTSAPPEYDINEYKDYFQWKYGLDKNADGSYTVNENKFNEIYKKLYSFTENDLANKFKVKCRNTYFIKYGFLKVPNLLDDDIYTVSEGFNIGNLAINNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLIVPYVGLALNIGNDVSKGDFKKAFELFGAAILLEVAPELLIPVILVFTIKSFIDSSENEDKIIKAIIKAINNSLMEREAKWQEIYGWIVSNWLTRINTQFNKRKEQMYQALQNQVDAIKTVIEYKYNNYTSDEKNRLESEYNINNIEEELNKKVSLAMKNIERFIAESSISYLMKLINEAKVSKLREYDEGVKEYLLDYILKHGSILGDRVQELNDLVTSTLNSSIPFELSSYTNDKILIIYFNKLYEKIKDNSILDMRYKNNKFIDISGYGSNISINGDVYIYSTNRNQFGIYSNKPSEVNIAQNNDIIYNSRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELDKTEIETLYSDEPDPSILKDFWGNYLLYNKRYYLLNLLRTDKSITQNSNFLNINQQRGVYQKPNIFSNTRLYTGVEVIIRKNGSTDISNTDDFVRKNDLAYINVVDHGVEYRLYADISIAKSEKIIKLIRTSNSNNSLGQIIVMDSIGNNCTMNFQNNNGGNIGLLGFHSNNLVASSWYYNNIRKNTSSNGCFWSFISKEHGWQE

SEQ ID NO: 47 - protein, 1297 aa, Clostridium argentinense
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 48 - protein, 1288 aa, Clostridium botulinum
MPVVINSFNYDDPVNDNTIIYIRPPYYETSNTYFKAFQIMDNVWIIPERYRLGIDPSLFNPPVSLKAGSDGYFDPNYLSTNTEKNKYLQIMIKLFKRINSKPAGQILLEEIKNAIPYLGNSYTQEEQFTTNNRTVSFNVKLANGNIVQQMANLIIWGPGPDLTTNKTGGIIYSPYQSMEATPYKDGFGSIMTVEFSPEYATAFNDISIASHSPSLFIKDPALILMHELIHVLHGLYGTYITEYKITPNVVQSYMKVTKPITSAEFLTFGGRDRNIVPQSIQSQLYNKVLSDYKRIASRLNKVNTATALINIDEFKNLYEWKYQFAKDSNGVYSVDLNKFEQLYKKIYSFTEFNLAYEFKIKTRLGYLAENFGPFYLPNLLDDSIYTEVDGFNIGALSINYQGQNIGSDINSIKKLQGQGVVSRVVRLCSNSNTKNSLCITVNNRDLFFIASQESYGENTINTYKEIDDTTTLDPSFEDILDKVILNFNEQVIPQMPNRNVSTDIQKDNYIPKYDYNRTDIIDSYEVGRNYNTFFYLNAQKFSPNESNITLTSSFDTGLLEGSKVYTFFSSDFINNINKPVQALLFIEWVKQVIRDFTTEATKTSTVDKLKDISLVVPYIGLALNIGDEIYKQHFAEAVELVGAGLLLEFSPEFLIPTLLIFTIKGYLTGSIRDKDKIIKTLDNALNVRDQKWKELYRWVVSKWLTTINTQFNKRKEQMYKALKNQATAIKKIIENKYNNYTTDEKSKIDSSYNINEIERTLNEKINLAMKNIEQFITESSIAYLINIINNETIQKLKSYDDLVRRYLLGYIRNHSSILGNSVEELNSKVNNHLDNGIPFELSSYTNDSLLIRYFNKNYGELKYNCILNIKYEMDRDKLVDSSGYRSRINIGTGVKFSEIDKNQVQLSNLESSKIEVILNNGVIYNSMYENFSTSFWIRIPKYFRNINNEYKIISCMQNNSGWEVSLNFSNMNSKIIWTLQDTEGIKKTVVFQYTQNINISDYINRWIFVTITNNRLSNSKIYINGRLINEESISDLGNIHASNNIMFKLDGCRDPHRYIWIKYFNLFDKELNKKEIKDLYDNQSNSGILKDFWGDYLQYDKPYYMLNLYDPNKYLDVNNVGIRGYMYLKGPRGRIVTTNIYLNSTLYMGTKFIIKKYASGNKDNIVRNNDRVYINVVVKNKEYRLATNASQAGVEKILSAVEIPDVGNLSQVVVMKSENDQGIRNKCKMNLQDNNGNDIGFIGFHQFNNIAKLVASNWYNRQIGKASRTFGCSWEFIPVDDGWGESSL

SEQ ID NO: 49 - protein, 1336 aa, Weissella oryzae
MDVLEMFDVNYESPILESFDSTTQSLNDVHVFMSRIQMSAYDADGEGRIEYRNLKLYEISSGIFISTDRLDTGASGVEDDHEMVDYYSSARLTREFLGESLDSQKSDYFEGIKKVFSFYKNKCNESRYIKEFFEEIQFRNICGFPKQAGTSSTDIFDQFNSVDVLLQDPVTSVWNKKVGSKKANIVIIPPATNLPITEACATAGFQPEGFPKLGSGSFFTVQFDPFFSTRFKAHETDDVALLDPTLTLLHEMTHGLHFQKGIANPVNRSGETPAWATTWGRVTGDNDAFKETPMEELLTFNKHTIDDDIEISDHLKSTYIGFLYNGRNEDDPTESVDGVYQNVSSFLNQYRGFEISSDFQHFIESCYGVKYNQESKKFIVNPRNIKRYVQDGFFIDEAKFARILNIKTRSYYTLMPDNLGVWSYRVDILNRLRETFDEDRGLLSQELDFHTALTPVVSENPALELEVAGMQRMVSLPKIKASYLPSDIKIKNFTGQKISHDTILDTNISGIIISKIKYKSDFVVDESMPRSSLNTTNYNLSPIKGTKFETDIRDKTSVKVTVSEITAPMINHVMKLDNSKVLTERPSLNEDLEETFKNTKDVYIPKTTAMMKLKEGADQTLGAVGFAVWSGQILEDLYNLAQKKEVSIDQIKDDLMSILPFYCAYKNLSAEKYEQAFANATLDAFLIFATDGGGFAGLGITVGAIAINSMYAKAETMEAYDSMFGKYVDQYQNDIKNFTLNAYVQWENNILSRLWNESRLAITGFRNMLKTVKTVMEFDATNQAYSEEDRKIIKAKCEEIFSEFPMLMQTFAKNSMTANLENASKIFNDIVWQKIKEELDQYVIDSKKYFLDSLEEAYNNGSISAESYYKYQTEAREKFVSPREVIDLYIAAHDTVVKRKRYIRRYSRKYDLATDFKGNTVHLNGLGEGTQDIQDLYGNYSVYADKKTVSTQEGHFDQTIKIAKDTNTINKVVLAVSSNNGKEYALNKDEQYTISFWLRMPVPSSSEERRIFSYSAVSGVNKEVEELILQVKNNEFVLATANLLRNSEFVIEPRIALNRWVKITIVNENTRIKVYQNDNLLGLIKDSSRKKPIAQRGTFKFYNYNVDYQLDDISYYNGTISQRDIKYTFKEDHGQFVYDHWGERLQYNKAYYLLSDDNKSAFETVYETKRLKLKSVPGVDIKYLGMNDRVYGYYGGLQFKLVPLDSKNMNNYVRWGDKFTMQSIETTNLSLAIIQDNAYFAPTQLKLISNEGKSEEEIFTFDRNIKLQNAAILVGTGNSKQGPISAYKRGYSGDLWINGARLDGYVTVVNKSNYSNDEIQEKFKWIFVPKDANWVE

SEQ ID NO: 50 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 51 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 52 - protein, 1279 aa, Enterococcus sp.
  source 1..1279: note = Enterococcus sp. 3G1_DIV0629
MVTINDLHYSDPIDEDNIINMRIPLYDLEVDDQFINHNVPDLKAFQVFPNVWVVPERYTFYSTMKNLDAPANPSRSSYYDPTYLQSDAEKEVFLQQMILLFKRINSTQEGQQFLNLLSRSIPVPYESNGDVAMGTTQVIKQMDDKGNVLKHRRAHIIIYGPGPDLMAKGSKALTKSRETGRGCMAEIYFSPMYHKTYSTKLTNKNSLVDKSVQEFVPDPAVTLIHELCHGLHALYGIDLGNVGSWEFNSNPNSLFSSWFSSKEAVNFEEVMTFGGEDVKVIKSEIDKKIPGILNLIKTTVEPIINKITDPHDEMLQCLQSKYPSLKGTLGQFFFDDTQLEKDIRDLWMVMNETMFAENLKALTRARYLVPKVENIVQVDILSPNVYTIDKGFNHLSKGFKGQSVSQSYFRKISALARGAVVRACPNPHFSSQRGLSSCIEILEDDLFIMSSKDSFTDTDFSEPSVGPVSYKAKKGADTILDSTLSNYDFSKEINFTSTVPIITVEDPLETDEDVPVISEDRTVYVDDYTTFHFLEAQKIGKEVVPTQTKVVFTTNMEEALFDSKKVYTVFENTASRINEAGTGIANGMMFYQWLKGIVQDFTEEATQKDTFDKISDVTMIVPYLGNILNIGNDIRKGDFMGAVELGGVTILLEAIPELTLPVLIGLTIIEDELEKEQVSQTVYNVLDKRDEKWEEVYGFVKQQWWWMVHTQFETRILHAYQALNHQVEAIKANMTYQLANYRGNQEDKELLEKAIDDTLQSLYYAVDQAMHNIKRFLIQSSKSYLLNQMLPKTKEQLLAFDQQTLRNVNDFINKNQGVLGESLAKDLKKKVEKRLTSLPVFNLEDLPISEFEDLIHSHEIDIQDSEVLNIGVNNGKIQDLSGENTPLTLGENLHIVNGRDNQAVRLNNQLDSKLEIQSRPNIHFTAFEDFSISIWIRCSMLRNNRNRGQKYTIIQQFNKYGWQLAIQDSVFVWTLHDTFNNQIQLTSGSALTNKNYLLQNFWLHITVTNKRSEKSRLYINGVLQDQKDISVLGNCHPKEPILFSIQDNSDPNYFVRFEQFNVYRKALTDSEVNRLYWKYFEGSYLRDVWGERLTYNRDYYMQLSTLPGRGIKREYRTWSGFDYIILSELGTQKIPTHEVTYPKLYQGQKITIHSDGKNLEPHVKSNKNIRLKIDDFYIGVVNPFKLPEWRPESGAYVVTTYNHAEDLCLYFRTRSSSQSLYYGQLIMNDGRNKSLLNYTLKGSTYWIWSSAWYYENYNTSSKTAGNWYFIPVDEGWKED

SEQ ID NO: 53 - protein, 1467 aa, Chryseobacterium piperi
MPFNYNDPIDDINVVSMKIPYAPFDSYEKAFQLKPGIYVLPQAMFDLDSGDTADSWLNFTRYNSIETKQEFLNTISDHLEDISRGRAGKTLLDAIENLKPLPVYYEGEPLGGNLIGYDPENTKIAYFDTEEGYFDIATKEGKNRKVGHVIIMGLSPGAIANGIYNQQKMQGSVEGYGRGMGSMISIDPAVTGKVMSTGAIIPSFGVLAHELCHSFQVLIEVEKANTMFYIEDQYHGPLRDKEGNIIIDNFTGREKYPVKEVRAVEVNVTNPAWRDFYRRIVDVVGISHAFDYWKYLLRRIDYLDSEVLVGFSDKANVLRKRLDYRIWNIYYNFPTEVEIIADSLTAKGYPIDYIQRFLRGSYLDFEGPYKVESLELDPSDSKIQKLNNVVSCDLIHSRKKRSITAYKRSISKLISCIPSITSQPANFVEKDSFFKDVEINELRRNSIKVVNEFLDSQKNTDQFKPYFVNSFKSAEKYPRAKIINFKNSVEVLNRGLTSVPMDVFEFYGWCKSLIEVFKSDADNFTKTASALGIVPIVGDLLGIADALRKEDYNAVASQIALLAVLGGAVVIGGPIGLTISAIIGIIFLGIYLKDLIEGIIHYFAPRDLNEEVQKYTRLRDRVWKSDVVKKVKEQIIPEMESSYRVFETQVRESLEISINLILKNAEEAKNAEKDLNIKNEIMATACGRVEEMYYIYDEMLKDVAGKTIRDCKEAVYQLVKEYHSHNYEKNSKDILEQYKKDYVAQYISWKTYEGTLESDRIQNRKEGNERFETIKSRVSKIEILKDQWSWLDECFQGKFWPDLIFSLPLLHIDLREAQKEQYALTWDLPDLPEELLALLRVEAYVLSKKTKIPSYWISTQMKDKKTDHLIPITDSWDAIMVYIKTPTALIGEHSYAKVKVNKNGDRYVDIEVNGRKSYLKDYLLPVPYNEYLFQYASSSLSLSSSDGSLIQEKYTSKGKQRWIFRKFRDNFYMIINAETKNKLTSNLDGTIGIQSGNIEENQAYKWEVVLDDEGYIKFRSGVYPDRFIGLADHSQTFSLVPDGKNCLFNLIKADAGRVEDKKAYTIFSEYDNRCITSLYASNSTSLPLGLWSLTSHPSQVWLANTVEESLNSFSFSNIFSNYFIRNNNGKAYQNSVVKIPFELYPLGEDSFLMFFEQRTCIMEGSSKEDKEGWELIAGMPSGNPKEKILLRRWRFGEFPSPGIYYNIVAFENFRKIKSDRYINIVQGDQEVKIESYQNRECLFRFEKQEAEYYFIYDSMNNLLTLSPIPGNRTRVSPKPFSKSDRNKQLWKVTYDDERKAWRIINKEVQLAIQAEDFENQGLKLGSRAEIDSGWQSWLIVEPEAFGLETEKKYVATIENGEQDMVLSWDESTSTAREKIYKINHSILDMSIEPRIFPVKYLGKEAPFITMLDFIFVYEKGHYKLFSKDYAGDCLTFIPDKDSTENIVFAKESQDNIKNQIWNITKIE

SEQ ID NO: 54 - protein, 1617 aa, Chryseobacterium piperi
MPILEVKPIYTDPYDDINVVKIELDDGVSVKAFKLTKNIFLSPEPSYIKDTEVTLSERDDYIQSMYGVDGDVKRQQFIDDIYYQMQMIQRGASGKLLIDEIDGLIPLPTAYGARYRKDDHENLFELQDYFIRNKIIDNIRNGLFKIEDGYFTQELQTGSDKGKIKKIGNVLIMAEHPRIEGKKKSMDLGYSFETGNAMFRNSFGSKSIISINPRTSQRLHSSGKIMSPWIRLAHELIHSHQRLLDVDLSGEMAKVRNYFGDLGPIPVGGSKNTPVIEIYNCNIEEKRRIYLEKIREYGEDRRKTSSDRIYNLIEKALALVTNITEEEKLNIQRRITYRKGLVEDFLNRPTEESIIYDTYMNTIDTQPEITSKRKDQIKKKIKDYSRKGYNDTTGKEYKLDAAKNPNFGSAEDLRDIPSCSYAKQKRAIGRGGGRLISTTAACISPNIPGEKISKEKLRNLYIDKSKYNKKTASIVSKANIDVKEIRKILTEGKVKFFFVDARQTYIPNTKVIIYEKTVRKINAGLNSKAMTVLDLYHWVKGIILAYTSDLDRFSKIVAYAGIIPVAGDLMQISEEIRHKNYSGAGFQTAVLSVFIGAALLAAVGSPVGIGILVIMGAAFAGLSIGRLIKDIIEWGKSKEDKQEKELIKYRDEVWKRMVKEVFNKNVVPEMENIYRTWEQGARESLELTINVIKAKTEHLERGEKAKIQIELEIDNMRKKFDDMLVGAVSQIKRSMKQELENLTRDYYAEYFKTNSEKIIDGLRSQLVKEFASTRLADQSWWGNKRGIIDMSMDQYLKLRQIIIDTPMSLKDLSWLDECINEQHFQPNFIFPTGPIRKLYVEYKIKDTSPSIIGSQNIVINWDCPKNIPKELGIAAKIEVREFNDDDRESHMISSELVWEKNYAIQFRNAKFIFTRSKFIRYEFSITPYTSVSPDQPGGETMILKLRFDRNLNTYGSSTINNGERPSDYDYLLPKPYNQYLIFDYYPDSVMAFRDKNDLTASIRQDVKSKENYMRCRFVPLKEDSYNSYHILINKDGEWRILELNEDSSKKAYSLRIRERKTSDEENHWRRWRIHFTQDGKINFSCIALPNKKLGRRHRSLSWTIEDLEDWSVTDFTLVLSGSEEIIDNKCYSIVSSQNNMAVTVSPFYPSSPITWSFDGNPTQLWLSEQADGEAWNFKNVHAQKYLEVEHEAQFEDLRIANIVLSEKNEKNENRLKIHVQGIDMRGLTKVTPYNNKSLVLDIFKQGDKKNEKGQKLIMFHEDIKERKKLDWVFSEFPALRTPYTISALKTLRILSGKDKLTVDNHDISEFQLASDKNNQWFFEKYTEDLYMIFNCGSGKALKQIKNKVGLRDTASLEVMPLKRDSSFRAEEYLWSMVYDDREEGWNIVSAGSDREAIRVSISEENKVAITISKPYGREYLFRIVPVKVDEILLEKEATYVISNADGLVITRSSKKELQDGNWLTGVIDAPLKMTPISYTTRFKPSSFQKWELKRIRAIYNDEKKYFWAFVNNIESYEEGNTKLYFFNSDPDNSPIRQASSHYFAFLIFYYGGKYMIKIEKKDESDLSLPVITYQPPEKEEEKISDVLFLKKEILDRNDSKLLSQLWYFTKLDSDDEF

SEQ ID NO: 55 - protein, 1496 aa, Chryseobacterium piperi
MRFDYNTPVDNINIVEMLIPYHPLNVYTKAFQIEPNIFIMPEARFGGYDVIENYLAFDRYGTVQAQQQFMDYVDGQLEQIKSGVAGKKLIEQLSNLAPIPNTDNAQGTTTFGDEGRTNLGLSIQNFDPNEGYFQIRINTSGDIENPIYSYKKVGHVLIMGLSPGNISRGAYNEHSSLKGIWGNGFGSGSIISIDPVVTIKVERTQNFQPPFSGLLHELCHSRQALLDIEYQNAVTALQRIDQFGRVEVSNVPVVEAYVTNSVDRYRIDTGQGLVYLPGIQDPIIYNLQTLFDMHRSDYDYMRTMVSVLAELDPEGWSIKSQEIHNRLVYRENLVGHAYTEITTEDMILADSLTALSGRPTYEVFTERRIMASFRRNYLDNEFITYRVNNPIHPDYTEAERLESIPSCSLRRNKRGIGSSLFNGLFLCRPGVISDDELKIYRKKKQEPLTIVKRKAKNLVEDFMKKRQVDNIFKPQFVDSYRIAKITPRSQVVIFKKTTKVLKQALKPSRMDALEIYNWGKNLIHVFDSDADDFTKASAALGIIPVVGDILGIIDDIRLGNAEQAIIGSFVLGALVTGAVIGGPVGVVIGAVVGIVFAIINIKSIIEDFINYVKKLQNLADPVNDFIGYRNESWKYYIVQVAKKEILPKMEEMYRIFESNARESLEIAVAVLEEKARLTVEKEPDEEAKKKILYTAHMQIEEMYDRYDDMLKEVAAKSIRDSKDLLHKMVREYYNQNFKKDSEEIIEGRRAKYVEEYHHLAFSAHKTDQKRELVNRKANEIFDNAKTKVKDTSMSLEDLSWLDECFEGLFWPEFINYSLPLLHISNIRAKEEFPDIANIDRIGTYTVDWSLLDDLPTELLNLLRVEIVMETDFSQSGWHRRPNGYESFSVPIMAQRIVIPRKEIKNRRNIFNYSADLHFRIRSPQGVENNETFIHDDIKKIGETSHHTYTFNTNGRANSKYAHLLPKSYYEYILSYNPSKFVLDSIGNELVSSNYFLKQSQKWKFKKHNDGTYLIRNTSNIGILSARGQTEVKFVVKSSEEFAEERWNIEIDDQGYQIRNYEVDNRFIGMNDSMKLDGIFGLIESENPNTYFNFIKEDSVVLKDGGIYRLFSEAGDYVLSLPFGISYTNEALCIWRNYHYAELWILKLRSNGVWYLRNIHSGKFIDFEPGDKLIHQNDVHHDHMDLDMNMIKGNVFMIVSNKKNNYAIDIAGNVIEPGRILISYQKKETKNQEWRFEPFPTVGIFYNILNMANWRKINSTEGKAQQGDNKAEINYYKNEKCLFRFELQEEGLYYVLDNENKLLRAPYLQREVIFEILNNELREEFLWEVSYDTLRGGWNIINKISGKAVQALASEGVNTNLIQADFKGTDSDFLSQSWMIAEPEAYGIEKEDYTRYIVTHEDKIVGYTKEKGYTATLVSDLVNREEFSQYPDESYQFNISDYIFQTANKGLFVIHSQESGNRILTYSKDSKTMSFEEIDPNNLDYIQKWNVTRIPLK

SEQ ID NO: 56 - protein, 1260 aa, Paraclostridium bifermentans
MLQIRVFNYNDPIDGENIVELRYHNRSPVKAFQIVDGIWIIPERYNFTNDTKKVPDDRALTILEDEVFAVRENDYLTTDVNEKNSFLNNITKLFKRINSSNIGNQLLNYISTSVPYPVVSTNSIKARDYNTIKFDSIDGRRITKSANVLIYGPSMKNLLDKQTRAINGEEAKNGIGCLSDIIFSPNYLSVQTVSSSRFVEDPASSLTHELIHALHNLYGIQYPGEEKFKFGGFIDKLLGTRECIDYEEVLTYGGKDSEIIRKKIDKSLYPDDFVNKYGEMYKRIKGSNPYYPDEKKLKQSFLNRMNPFDQNGTFDTKEFKNHLMDLWFGLNESEFAKEKKILVRKHYITKQINPKYTELTNDVYTEDKGFVNGQSIDNQNFKIIDDLISKKVKLCSITSKNRVNICIDVNKEDLYFISDKEGFENIDFSEPEIRYDSNVTTATTSSFTDHFLVNRTFNDSDRFPPVELEYAIEPAEIVDNTIMPDIDQKSEISLDNLTTFHYLNAQKMDLGFDSSKEQLKMVTSIEESLLDSKKVYTPFTRTAHSVNERISGIAESYLFYQWLKTVINDFTDELNQKSNTDKVADISWIIPYVGPALNIGLDLSHGDFTKAFEDLGVSILFAIAPEFATISLVALSIYENIEEDSQKEKVINKVENTLARRIEKWHQVYAFMVAQWWGMVHTQIDTRIHQMYESLSHQIIAIKANMEYQLSHYKGPDNDKLLLKDYIYEAEIALNTSANRAMKNIERFMIESSISYLKNNLIPSVVENLKKFDADTKKNLDQFIDKNSSVLGSDLHILKSQVDLELNPTTKVAFNIQSIPDFDINALIDRLGIQLKDNLVFSLGVESDKIKDLSGNNTNLEVKTGVQIVDGRDSKTIRLNSNENSSIIVQKNESINFSYFSDFTISFWIRVPRLNKNDFIDLGIEYDLVNNMDNQGWKISLKDGNLVWRMKDRFGKIIDIITSLTFSNSFIDKYISSNIWRHITITVNQLKDCTLYINGDKIDSKSINELRGIDNNSPIIFKLEGNRNKNQFIRLDQFNIYQRALNESEVEMLFNSYFNSNILRDFWGEPLEYNKSYYMINQAILGGPLRSTYKSWYGEYYPYISRMRTFNVSSFILIPYLYHKGSDVEKVKIINKNNVDKYVRKNDVADVKFENYGNLILTLPMYSKIKERYMVLNEGRNGDLKLIQLQSNDKYYCQIRIFEMYRNGLLSIADDENWLYSSGWYLYSSGWYLDNYKTLDLKKHTKTNWYFVSEDEGWKE

SEQ ID NO: 57 - protein, 1230 aa, Paeniclostridium ghonii
MIPINIKDFNYSDPVNNQDIILVKNEKGSFDKGFFVADKILLVPARYGNISTDEGGITSKKEKAHVDKKIYLETDSEKNEYLKNMTTLLKRMNSYSTGNKLLNLIIKGEPIYSKDLQGKFIEQTPSRYLDTNTGKRRVNVMITGPGSNVLTKKCTHNGMGLENDPNGKHSNGTGILSTIEFSPNYLIAYNKCVADPVLTLFHELVHSMHNLYGIAFPDNVKVPYNALKDKNLVSGEEALSEILTFGGKDLTTEHLETLWKKLAETVIIVKDFVKTDTQAKDVFLNNLRFLSKNENIKIDTIEDIVNGTLKIKNNISNLTECEFCKEIGDVRIRTRYAVHSEDVTPVEVVDFKNNYKLNSGFLEGQDISKKYFITNPPKMRRRALRNFKCTIQMADIIASVDKKDVFAVSDTSYFKNFKFPSKKISDTGEVIDSTKLPQIKDTYKSSREEPIPDNDSTINVKNITTYHYLEAQKPKNSSIELTMVAPSKSKKPNDCVVEAINDNNKIYTPFSGTAKQFNTVVPIANTAANVITWLEAIADIFSSETGTFDKLERAGKETLYYIPYVGQLLSIGENVLIGDFKNALLNTGLIILLDIAPELNIPLLGAFEAYKEYKSLEEFRKAIDNVIDERNKRWHSVYSFVAHQWYGQVNIQIEQRLNHFYQALSYQAGVIKNRVDIEYARHKEGLEEKEERKLMWASVDCIGSIEASVKEATKNAEKFLEKSSILYFKEEILPKVHKNLEEFDKNTLFNIYTNIDEFSNRGIAEISECKKVEADVNNGFRPIKFDFSLLTNLMKSDSLTDEVILEKALEDALVFSLGVRNGKIQNLSKKWANLTIGTDIRVVHGRDNESIRLNSTQDSSIQIEKNTNLRFLDSENFSLSFWIRVPRYNKFDKDKDLNNEYTIVNNMDTATKGFKISIKNGILLWTLKGTQQKTIEIPLSNTKVSDNIWRHVAIINNKDGNCTIYVDGAQKNAVSLSGLDEITNTLPITLQLVGNKNKKQFIRLDQFNIYEKALSQTEVGKLFSSYFKDSDIRDYWGEPLAYNKTYNMINIAYQGRGLQSTNNKISLQPKAVFDPTGDGSYIPRLYRGYDVLLQKDSQSKTTDIMPKKDDLINIKLKSGHNFVGFNSTIDTSQKYLKLTTALLSEVDDPKGFKLMSLKKDNWIQIKKETWMSKNGNVIPQGLVGKRSVDSDVYLYLWDWETEKDDYSEKQWSFICQDEGWIDSDGMFTNA

(19) United States
(12) Patent Application Publication
(10) Pub. No.: US 2026/0356895 A1
(43) Pub. Date: Nov. 12, 2026

(54) USE OF AN ATOXIC C. TETANI STRAIN AS A UNIVERSAL EXPRESSION SYSTEM FOR COMPLEX-FREE BONTS

(71) Applicant: Wisconsin Alumni Research Foundation, Madison, WI (US)

(72) Inventors: Sabine Pellett, Madison, WI (US); William Tepp, Stoughton, WI (US); Brieana Gregg, Madison, WI (US)

(21) Appl. No.: 19/391,241

(22) Filed: Nov. 17, 2025

Related U.S. Application Data
(60) Provisional application No. 63/721,483, filed on Nov. 16, 2024.

(51) Int. Cl. C07K 14/33; C12N 15/74

(52) U.S. Cl. CPC C07K 14/33; C12N 15/74

(57) ABSTRACT

The present invention provides atoxic Clostridium bacteria that are engineered to express a botulinum neurotoxin (BoNT). Methods of making these bacteria and methods of using these bacteria to produce a BoNT are also provided, as are expression vectors that encode a BONT and can be used to make these bacteria.

### CROSS-REFERENCE TO RELATED APPLICATIONS

The present application claims priority to U.S. Provisional Patent Application No. 63/721,483 that was filed Nov. 16, 2024, the entire contents of which are hereby incorporated by reference.

### STATEMENT REGARDING FEDERALLY SPONSORED RESEARCH

This invention was made with government support under grant numbers AI139306 and AI118389 awarded by the National Institutes of Health. The government has certain rights in the invention.

### SEQUENCE LISTING

A Sequence Listing accompanies this application and is submitted as an xml file of the sequence listing named “960296_04730.xml” which is 220,722 bytes in size and was created on Jun. 29, 2026. The sequence listing is electronically submitted via Patent Center and is incorporated by reference herein in its entirety.

### BACKGROUND

Botulinum neurotoxins (BoNTs) are a family of closely related protein toxins and are the agents responsible for botulism, a rare but extremely serious disease characterized by descending flaccid paralysis [1,13]. BoNTs are produced by several species of Clostridium, including Clostridium botulinum, and have been traditionally categorized into seven immunogenically distinct serotypes (BoNT/A-G), which contain over 45 subtype variations and mosaic toxins [14,15].

Recombinant BoNT production is an invaluable tool for functional characterization of BONTs for basic research, for vaccine development, and for the development of novel engineered pharmaceutical BoNTs. However, several BoNTs are produced by their native C. botulinum strains in quantities that are insufficient for structural and functional analysis, and no optimized expression system exists for production of mutated or chimeric BoNTs. Current methods for rBoNT expression rely either on E. coli, which lacks the ability to proteolytically process and activate the toxin, or atoxic C. botulinum type A strains, which retain native type A neurotoxin associated proteins (NAPs) and have only successfully been used to produce BoNT/A serotypes. Thus, there is a remaining need in the art for expression systems that can be used to produce other BONT serotypes and variants.

### SUMMARY

Provided here are means of producing a BoNT in an atoxic Clostridium tetani such that the BONT can be produced without the associated neurotoxin-associated proteins (NAPs) to maintain stability of the BoNTs. To generate BoNTs, expression vectors comprising a promoter operably linked to a polynucleotide encoding a botulinum neurotoxin (BoNT) are provided. An atoxic Clostridium bacterium engineered to express a heterologous BONT and the atoxic Clostridium may be a Clostridium tetani, as provided herein.

In another aspect, methods of making the bacterium described herein are also provided and include introducing a polynucleotide encoding the BoNT into the bacterium.

In another aspect, methods of producing a BoNT are provided. The methods include culturing the bacterium provided herein in a culture medium; and collecting the BoNT from the culture. The BoNTs produced using these methods are not complexed with the neurotoxin-associated proteins.

In another aspect, methods of using the BoNTs produced by the disclosed methods to treat a condition are also provided herein. The conditions may include a pain condition, movement disorder, neurological disorder, neuromuscular disorder, spasticity, cosmetic issue, axillary hyperhidrosis, migraine, cervical dystonia, blepharospasm, strabismus, achalasia, upper motor neuron syndrome, sweating, or wrinkles.

### BRIEF DESCRIPTION OF THE DRAWINGS

FIG. 1 demonstrates that C. tetani 454 and C. tetani 19406 express stable, full-length recombinant TeNT (rTeNT) at levels exceeding that of a wild-type strain under the ferredoxin promoter. Western blot of rTeNT expression by C. tetani 454 and C. tetani 19406 cultured in modified Mueller Miller media (supplemented with 0.5 g/L reduced iron powder) for 24-96 hours. Expression construct pMTL82153-TeNT contains the ferredoxin (fdx) promoter, while pMTL82155-TeNT contains the C. tetani positive regulator tetR, as well as the endogenous tetR promoter (p_{tetR}) and the tent promoter (pₜₑₙₜ) regions. To maintain the recombinant plasmids, the culture media was supplemented with 15 μg/mL thiamphenicol. Wild-type C. tetani WR23 was used as an expression control. All samples are unreduced, whole culture.

FIG. 2 demonstrates that the catalytically inactive BoNT/A1 mutant rBoNT/A1^{ERY} is expressed by C. tetani 454 and is partially proteolytically processed to its dichain form but also degraded over time. A: Western blots depicting the stability of rBoNT/A^{ERY} over time when the expression strains are cultured in toxin production medium (TPM). C. botulinum Hall A hyper/tox-(82152-A1^{ERY}) is known to strongly express stable BoNT/A and was therefore used as an expression control to compare against C. tetani 454 (82153-A1^{ERY}). B: Western images demonstrating partial proteolytic activation of rBoNT/A1^{ERY} produced in C. tetani after 144 h of culture in TPM. Whole culture samples were collected at the noted times, and either reduced with 100 mM DTT or left unreduced. Reduction separates the disulfide linked LC and HC if the single chain pro-toxin has been proteolytically activated by cleavage between LC and HC. 50 ng purified BoNT/A1 was used as a loading control, and rBoNT/A1^{ERY} produced in C. botulinum strain Hall A hyper tox− is shown as a comparison ibn the left side panel. The locations of the ~150 kDa holotoxin (HC+LC/A), the ~100 kDa heavy chain (HC/A), and the ~50 kDa light chain (LC/A) are indicated.

FIG. 3 demonstrates that rBoNT/A1^{ERY} is expressed by C. tetani 454 in modified Mueller Miller medium. (A) Western blots of C. botulinum Hall A hyper/tox-(83152-A1ERY) and C. tetani 454 (82153-A1ERY) cultured in modified Mueller Miller media (+0.5 g/L reduced iron powder). Whole culture samples were taken at 24 and 96 hours, and either reduced with 100 mM DTT or left unreduced. (B) Western blot of C. tetani 454 (82153-A1ERY) expression on modified Mueller Miller agar plates without the addition of reduced iron powder or any alternative iron source. Whole cell (WC) growth on the plate was resuspended at the indicated time points and centrifuged into cell pellet (pellet) and supernatant (SN). 50 ng purified BoNT/A1 was loaded as a control. The locations of the ~150 kDa holotoxin (HC+LC/A), the ~100 kDa heavy chain (HC/A), and the ~50 kDa light chain (LC/A) are indicated.

FIG. 4 shows the stability of Clostridial neurotoxins in spent media under varying pH conditions. Static cultures of the atoxic strains C. tetani 454 and C. botulinum LNT01 were incubated at 37 °C for 96 hours in either modified Mueller Miller media (supplemented with 0.5 g/L of reduced iron powder) or toxin production media (TPM). Culture supernatant was collected, and pH was adjusted to pH 5, 6, 7, and 8 via 1M NaOH or 1M HCl. The pH adjusted spent media, spent media with no pH adjustment, and media which had not supported bacterial growth (Fresh Media) were sterile filtered through a 0.22 μM PVDF membrane. Purified 150 kDa BoNT/A1 or TeNT were added to each sample, incubated for 24 hours at 37 °C, and then analyzed via SDS-PAGE and western blot. The ~150 kDa protein bands, indicating the amount of full-length toxin remaining, were analyzed via densitometry. The mean concentration (n=3) of remaining holotoxin is represented as a percentage relative to the amount of toxin initially added to the sample.

FIGS. 5A and 5B show the selection of optimal promoter and culture media for expression of recombinant toxins in C. tetani. C. tetani 454 and C. tetani 19406 were conjugated with pMTL82153-TeNT or pMTL82155-TeNT and cultured statically at 37 °C in the noted media alongside the wild-type C. tetani strains 64001 and WR23. 15 μg/mL thiamphenicol was added to the growth media for maintenance of the pMTL-TeNT plasmids. After 24 hour FIG. 5A and 96 hour FIG. 5B of culture, unreduced samples were analyzed by SDS-PAGE and western blot. 50 ng TeNT 8MTT served as a positive control. The samples from left to right in each set of three lanes on the gel are MM, HS, HS-T, wherein MM: modified Mueller Miller (+reduced iron); HS: HySoy (+reduced iron); HS-T: HySoy-T (+reduced iron).

FIG. 6 shows representative western blots of BoNT/A1 and TeNT stability in spent media. Three replicate western blots per sample condition were analyzed via densitometry to calculate the concentration of ~150 kDa holotoxin remaining after 24 hours of incubation in spent C. botulinum LNT01 or C. tetani 454 media. The mean percentage of remaining full-length toxin is depicted in FIG. 4. These western blots indicate the extent and size of breakdown products under varying pH conditions.

### DETAILED DESCRIPTION

The present invention provides atoxic Clostridium bacteria that are engineered to express a botulinum neurotoxin (BoNT) in the absence of the non-toxic stabilizing proteins. Methods of making these bacteria and methods of using these bacteria to produce a BoNT are also provided.

Tetanus neurotoxins (TeNT) and botulinum neurotoxins (BoNTs) are closely related toxins that are expressed by Clostridia bacteria. While TeNT, which is expressed by the bacterium Clostridium tetani, is expressed as a single polypeptide, BoNTs, which are expressed by the bacterium Clostridium botulinum, are always produced alongside multiple non-toxic proteins (i.e., neurotoxin-associated proteins) that form a stabilizing complex with the BoNT. These complexing proteins appear to be essential for protecting the BoNT from degradation in culture supernatants of C. botulinum, and they make recombinant expression of BoNTs challenging.

The present inventors have developed a universal BONT expression system that utilizes an atoxic C. tetani strain as the expression host and circumvents the need to co-produce BONT complexing proteins. In the Examples, the inventors demonstrate that a catalytically inactive BoNT/A1 mutant (BoNT/A1^{ERY}) can be expressed complex-free at high levels in C. tetani, and that the resulting protein is stable and partially proteolytically processed, like the BoNT/A complex produced endogenously by C. botulinum. Their data suggests that the metabolic state of C. tetani, which is affected by growth conditions such as the culture medium, is a primary factor in creating a favorable environment for complex-free neurotoxin stability.

### Botulinum Neurotoxins (BoNTs):

A “botulinum neurotoxin (BoNT)” or “botulinum toxin” is a neurotoxic protein produced by the bacterium Clostridium botulinum and related species. BoNTs are a highly sought after pharmaceutical agents for treating both cosmetic conditions (e.g., smoothing fine lines and wrinkles) and medical conditions (e.g., muscle over-activity, chronic pain). BoNTs induce their effects by blocking the release of the neurotransmitter acetylcholine from axon endings at the neuromuscular junction, which prevents muscles from firing and causes flaccid paralysis.

BoNTs are categorized into seven immunogenically distinct serotypes, namely type A (BoNT/A), type B (BoNT/B), type C (BoNT/C), type D (BoNT/D), type E (BoNT/E), type F (BoNT/F), or type G (BoNT/G). These serotypes contain over 45 subtype variations. BoNTs belonging to any serotype may be produced using the expression systems described herein. BoNT amino acid sequences are provided herein as SEQ ID NOs: 7-50. Additional sequences may be used in the context of the disclosed compositions and methods, e.g., a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to any one of the disclosed sequences, e.g., SEQ ID NOs: 7-50.

Currently, only BoNT/A1 (Botox, Xeomin, Dysport) and BoNT/B1 (Myobloc) are used as pharmaceuticals, and optimized expression systems for producing BoNTs at scale via fermentation are only available for wild-type BoNT/A and BoNT/B serotypes. To address this limitation, the inventors have developed a universal BoNT expression system that does not require the expression of serotype-specific complexing proteins and can therefore be used to produce engineered BoNTs and BoNTs of any serotype or subtype. Thus, in some embodiments, the BoNT produced by the expression system is an engineered BONT or a BoNT of a serotype for which no expression system is currently available, i.e., BoNT/C, BoNT/D, BONT/E, BoNT/F, or BoNT/G.

An “engineered BONT” is a BoNT that has been altered by the hand of man. Engineered BoNTs comprise one or more mutations relative to a wild-type BoNT. The term “wild-type” is used herein to describe the non-mutated version of a BoNT that is most typically found in nature. The engineered BoNTs used with the present invention may be derived from one or multiple wild-type BoNTs. For example, the BoNTs may be chimeric BoNTs that comprise portions of two or more BoNTs of different serotypes or subtypes.

The term “mutation” refers to a difference in the amino acid sequence of the engineered BONT relative to the corresponding wild-type BoNT(s), i.e., the BoNT(s) from which the engineered BONT was derived. Mutations include insertions, deletions, and substitutions. An “insertion” is an addition of one or more amino acid residues relative to a wild-type sequence. An insertion may add 1, 2, 3, 4, 5, 10, 20, 30, 40, 50, 60, 70, 80, 90, 100, 150, 200, or more amino acid residues to a sequence. A “deletion” is a removal of one or more amino acid residues relative to a wild-type sequence. A deletion may remove 1, 2, 3, 4, 5, 10, 20, 50, 100, 200, or more amino acids residues from a sequence. A “substitution” is a replacement of one amino acid in a wild-type sequence with a different amino acid. A substitution may be conservative (i.e., a replacement with an amino acid that has similar properties) or radical (i.e., a replacement with an amino acid that has different properties). Methods for introducing a mutation into a deaminase-encoding polynucleotide include, but are not limited to, site-directed mutagenesis, oligonucleotide-directed mutagenesis, homologous recombination (in vivo or in vitro), total gene synthesis, and gene editing methods (e.g., CRISPR-Cas9-based methods).

### Expression Vectors:

In a first aspect, the present invention provides expression vectors comprising a promoter operably linked to a polynucleotide encoding a BoNT. The expression vector and system provided herein allow for expression of engineered BoNT without the complexing or stabilizing proteins.

The term “vector” refers to a DNA molecule that is used to carry a particular DNA segment (i.e., a DNA segment included in the vector) into a host cell. Some vectors are capable of autonomous replication in a host cell (e.g., bacterial vectors that include an origin of replication and episomal mammalian vectors). Other vectors can be integrated into the genome of a host cell such that they are replicated along with the host genome (e.g., viral vectors and transposons). Vectors may include heterologous genetic elements that are necessary for propagation of the vector or for expression of an encoded gene product. Suitable vectors include plasmids (i.e., circular double-stranded DNA molecules) and viral vectors.

An “expression vector” is a vector that comprises a polynucleotide operatively linked to a regulatory element (e.g., a promoter) that drives expression of a gene product (i.e., protein or functional RNA molecule) encoded by the polynucleotide in a cell. Expression vectors may contain one or more polynucleotide/regulatory element combination. The expression vector may also include additional sequences (e.g., a sequence encoding a signal peptide or a tag) that modify the gene produce encoded by the polynucleotide.

In some embodiments, the expression vector is a shuttle vector. A “shuttle vector” is a vector that can propagate in two different host organisms and allows for the transfer of genes between two or more organisms via conjugation. For example, some shuttle vectors can propagate in different species of bacteria, and some shuttle vectors can propagate in both eukaryotes and prokaryotes. The use of a shuttle vector allows the vector to be manipulated and/or propagated in one organism (e.g., E. coli), and then transferred into another organism that is more challenging to manipulate/propagate (e.g., Clostridium tetani) for protein expression. The expression need not be a shuttle vector and the vector can be transferred to C. tetani using electroporation.

As used herein, the term “promoter” refers to a DNA sequence that defines where transcription of a polynucleotide begins. RNA polymerase and the necessary transcription factors bind to the promoter to initiate transcription. Promoters are typically located directly upstream (i.e., at the 5′ end) of the transcription start site. However, a promoter may also be located at the 3′ end, within a coding region, or within an intron of a gene that it regulates. Promoters may be derived in their entirety from a native or heterologous gene, may be composed of elements derived from multiple regulatory sequences found in nature, or may comprise synthetic DNA. A promoter is “operably linked” to a polynucleotide if the promoter is positioned such that it can affect transcription of the polynucleotide. In the Examples, the inventors tested several promoters in their BONT expression system, including a ferredoxin promoter, a thiolase promoter, and a tent promoter. Thus, in some embodiments, the promoter is a ferredoxin, thiolase, or tent promoter. However, the inventors achieved the highest levels of BONT expression using a ferredoxin promoter. Thus, in preferred embodiments, the promoter is a ferredoxin promoter.

The ferredoxin promoter may comprise SEQ ID NO: 3 or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 3.

The thiolase promoter may comprise SEQ ID NO: 4 or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 4.

The tent promoter may comprise SEQ ID NO: 5 or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 5.

In some embodiments, the expression vectors further comprise a selectable marker. As used herein, the term “selectable marker” refers to a polynucleotide that encodes a gene product that protects an organism from a “selection agent,” i.e., an agent that would normally kill it or prevent its growth. For example, in some embodiments, the selectable marker confers resistance to an antibiotic (e.g., puromycin, penicillin, streptomycin, neomycin, hygromycin). Cells that comprise a vector that includes a selectable marker can be exposed to the corresponding selection agent to select for cells that comprise the vector. In the Examples, the inventors included the antibiotic resistance gene catP, which provides resistance to the antibiotic thiamphenicol, in their expression vectors. Thus, in certain embodiments, the selectable marker is catP, e.g., SEQ ID NO: 6, or a sequence with at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or about 100% identity to SEQ ID NO: 6.

The terms “polynucleotide,” “nucleic acid,” and “oligonucleotide” are used interchangeably to refer a polymer of DNA or RNA. A polynucleotide may be single-stranded or double-stranded and may represent the sense or the antisense strand. A polynucleotide may be synthesized or obtained from a natural source. A polynucleotide may contain natural, non-natural, or altered nucleotides, as well as natural, non-natural, or altered internucleotide linkages (e.g., phosphoroamidate linkages, phosphorothioate linkages).

### Bacteria:

In a second aspect, the present invention provides atoxic Clostridium bacteria that are engineered to express a heterologous BONT.

Clostridium is a genus of anaerobic, Gram-positive bacteria. This genus includes the species Clostridium tetani, which produces tetanus neurotoxins (TeNT), and the species Clostridium botulinum, which produces BoNTs. In the Examples, the inventors demonstrate that BoNTs can be stably expressed in C. tetani. Thus, in preferred embodiments, the bacterium is C. tetani.

The bacteria of the present invention are “atoxic,” meaning that they do not produce any substances that are considered toxic to humans. Any Clostridium strain that does not naturally produce a toxin or that has been engineered to prevent it from producing toxins may be utilized. In the Examples, the inventors utilized two strains of atoxic C. tetani: C. tetani ATCC 454 and C. tetani ATCC 19406. Thus, in certain embodiments, the bacterium is one of these specific C. tetani strains. C. tetani ATCC 19406 lacks the entire 74 kb plasmid that encodes the tetanus toxin, whereas C. tetani ATCC 454 lacks just the tetanus toxin gene (tent) and its regulator and promoter region (tetR).

The Clostridium bacteria of the present invention are engineered (i.e., altered by the hand of man) to express a heterologous BoNT, i.e., a BoNT that is not natively expressed by that specific bacterium. The BoNT expressed by the bacteria may be of any serotype/subtype and may be engineered, as described above. Methods for producing such bacteria are described in the following section. In some embodiments, the bacterium is engineered to comprise an expression vector described herein.

BONT genes are naturally found clustered with genes that encode neurotoxin-associated proteins (NAPs), which are nontoxic proteins that form complexes with BoNTs that stabilize them against chemical and enzymatic degradation. NAPs are required for stable BONT protein production in Clostridium botulinum. In the Examples, the inventors demonstrate that BoNTs can be stably expressed in C. tetani in the absence of NAPs, and that the resulting BONT protein is partially proteolytically processed. Thus, in some embodiments, the bacterium produces active BONT in the absence of NAPs. As used herein, “active BONT” is BONT that is not degraded and has been proteolytically processed into its active dichain form.

### Methods of Making Bacteria:

In a third aspect, the present invention provides methods of making a bacterium described herein. The methods comprise introducing a polynucleotide encoding a heterologous BONT into the bacterium.

As used herein, the term “introducing” describes a process by which an exogenous polynucleotide is introduced into a recipient cell. Suitable introduction methods include, without limitation, (a) transformation, wherein bacteria directly take up DNA from their environment; (b) transduction, wherein a bacteriophage is used to transfer DNA into the bacteria; (c) conjugation, wherein DNA is transferred via cell-to-cell contact between donor bacteria and recipient bacteria; (d) electroporation, wherein an electric field is used to create temporary pores in the cell membrane to allow DNA to enter; (e) protoplasts, wherein the bacterial cell wall is enzymatically digested to allow DNA to enter; (f) microinjection, wherein DNA is physically injected into cells using a fine needle; or (g) gene gun bombardment, wherein high-velocity particles coated with DNA are shot into cells.

In the Examples, the inventors introduced BoNT-encoding vectors into C. tetani via conjugation with a donor strain. Thus, in some embodiments, the polynucleotide is introduced via conjugation with a donor strain. As used herein, a “donor strain” is a bacterial strain that can transfer genetic material to another bacterial cell via conjugation. Donor strains may include specific plasmids, called “conjugative plasmids,” that encode machinery required for conjugation. Engineered strains of Escherichia coli are most often used as donor strains. Thus, in some embodiments, the donor strain is Escherichia coli, e.g., E. coli CA434, a recA+ strain.

In some embodiments, the polynucleotide introduced into the bacterium is part of an expression vector. In some embodiments, the expression vector is an expression vector described herein.

### Methods of Producing a BoNT:

In a fourth aspect, the present invention provides methods of producing a BoNT. The methods comprise (a) culturing a bacterium described herein in a culture medium; and (b) collecting the BoNT from the culture.

“Culturing” refers to a method in which cells are grown in an artificial environment. Cells are typically cultured in a vessel (e.g., a dish, flask, plate, a fermentation tank, a bioreactor or tube) comprising a culture medium. The combination of the cultured cells, substances secreted by the cultured cells, and the media is referred to as a “culture”. Culturing involves providing essential nutrients to the cells, maintaining conditions (e.g., temperature, humidity, airflow, barometric pressure, percent oxygen, percent carbon dioxide) that are suitable for growth and reproduction of the cells, and then waiting for a period of time to allow the cells to grow and divide (e.g., for an hour, a day, or a week). For example, in some embodiments, culturing is performed at about 22 °C to about 30 °C. In some embodiments, culturing involves agitating or shaking the culture at about 150 to 220 revolutions per minute (rpm). In some embodiments, culturing is performed for at least 12 hours, at least 18 hours, at least 24 hours or longer periods of time including about 1-14 days, suitably about 2-10 days, or 3-7 days. However, the inventors have observed that, after 4-6 days of culture, C. tetani bacteria lyse and release BONT into the culture medium. Thus, in preferred embodiments, the bacterium is cultured for 4-6 days prior to step (b).

As used herein, a “culture medium” or “medium” is a composition designed to support the growth of a cultured cell. A culture medium may be a solid, liquid, or semi-solid composition. Typically, a culture medium comprises a carbon source, a nitrogen source, and micronutrients. Examples of suitable carbon sources include carbohydrates and alcohols. Examples of suitable nitrogen sources include amino acids, peptides, and ammonium salt. Examples of suitable micronutrients include trace metals, cofactors, vitamins, and essential salts. A medium may also comprise a salt used for osmolarity balance (e.g., sodium, chloride, potassium) and/or a pH buffering agent (e.g., a phosphate). In the Examples, the inventors demonstrate that BoNTs exhibit enhanced stability in a modified Mueller Miller medium. Thus, in some embodiments, the culture medium is a modified Mueller Miller medium. Other defined media may also be acceptable for use in the methods of making BoNTs provided herein. Defined media are used to control the metabolism of the expression host such that the NAPs are not needed to protect the BONT from degradation.

In step (b) of the methods, the BoNT is collected from the culture. As is noted above, the inventors observed that, after 4-6 days of culture, the C. tetani bacteria lyse and release BONT into the culture medium. Thus, collecting the BoNT may be as simple as removing the culture medium from the culture vessel. However, collecting the BoNT may further comprise lysing the bacteria in the culture if they have not lysed on their own. The terms “lysis” and “lyse” are used to describe the disruption of cells within a sample to gain access to materials within the cells. Lysis breaks the cellular membrane, causing the cell contents to spill out. Methods of lysis include, but are not limited to, chemical lysis, thermal lysis, mechanical lysis, and osmotic lysis.

Collecting the BoNT may also further comprise removing the bacterium from the culture medium to produce a cell-free culture supernatant comprising the BoNT. A “cell-free culture supernatant” is a liquid in which a bacterium was grown and then removed. Cell-free culture supernatant is produced by: (a) inoculating a medium with the bacterium, (b) culturing the inoculated medium, and (c) removing the bacterium from the medium. Cell-free culture supernatant comprises residual nutrients from the medium as well as enzymes, metabolites, growth factors, and extracellular matrix proteins that were secreted by the bacterium prior to its removal.

Methods for removing bacteria from a culture include, but are not limited to, centrifugation and filtering. “Centrifugation” is a method of separating substances of different densities by spinning them in solution around an axis at high speed. Centrifuging a vessel of a solution comprising bacteria at an appropriate speed will cause the bacteria to be compacted into a pellet at the bottom of the vessel, allowing the supernatant to be carefully removed from the vessel while leaving the pellet behind. Suitable centrifuge conditions for pelleting bacteria are known in the art and include speeds ranging from 1,000 g to 12,000 g and times ranging from 1 minute to 1 hour. “Filtering” is a method in which a substance is passed through a filter. A “filter” is a porous physical barrier that is permeable to some substances but impermeable to others due to their physical size. Any filter that has a pore size that is smaller than the size of the C. tetani bacterium can be used to remove the bacteria from the culture.

In some embodiments, the methods further comprise purifying the BoNT from the cell-free culture supernatant. As used herein, the term “purifying” refers to the process of separating a BoNT from other culture components and impurities. Suitable methods for purifying BoNTs include, without limitation, filtering, dialysis, affinity chromatography, ion-exchange chromatography, high performance liquid chromatography (HPLC), chemical precipitations, size exclusion chromatography, nanoparticle-based purification methods and any other method known or available to those of skill in the art. A “purified” BoNT may be at least 85% pure, at least 95% pure, or at least 99% pure.

### Compositions Comprising BoNTs:

In a fifth aspect, the present invention provides compositions comprising BoNTs. In some embodiments, the compositions comprise a BoNT produced by the methods described in the previous section. In some embodiments, the compositions comprise an engineered BoNT. In some embodiments, the BoNT is complex-free (i.e., not associated with neurotoxin-associated proteins).

In some embodiments, the compositions are pharmaceutical compositions, i.e., a substance that comprises a pharmaceutical agent that is suitable for administration to a subject. Pharmaceutical compositions may include a pharmaceutically acceptable carrier. The term “pharmaceutically acceptable carrier” refers to any carrier, diluent, or excipient that is compatible with the other ingredients of a formulation and is not deleterious to the subject to which it is administered. Pharmaceutically acceptable carriers are known in the art and include, but are not limited to, diluents (e.g., Tris-HCl, acetate, phosphate), preservatives (e.g., thimerosal, benzyl alcohol, parabens), solubilizing agents (e.g., glycerol, polyethylene glycerol), emulsifiers, liposomes, nanoparticles, and adjuvants. Pharmaceutically acceptable carriers may be aqueous or non-aqueous solutions, suspensions, or emulsions. Examples of nonaqueous solvents are propylene glycol, polyethylene glycol, vegetable oils (e.g., olive oil), and injectable organic esters (e.g., ethyl oleate). Aqueous carriers include isotonic solutions, alcoholic/aqueous solutions, emulsions, and suspensions, including saline and buffered media.

The compositions of the present invention may further include additives such as albumin or gelatin to prevent absorption to surfaces, detergents (e.g., Tween 20, Tween 80, Pluronic F68, bile acid salts), antioxidants (e.g., ascorbic acid, sodium metabisulfite), bulking substances or tonicity modifiers (e.g., lactose, mannitol). Components of the compositions may be covalently attached to polymers (e.g., polyethylene glycol), complexed with metal ions, or incorporated into or onto particulate preparations of polymeric compounds (e.g., polylactic acid, polyglycolic acid, hydrogels, etc) or onto liposomes, microemulsions, micelles, milamellar or multilamellar vesicles, erythrocyte ghosts, or spheroplasts. The compositions may also be formulated in lipophilic depots (e.g., fatty acids, waxes, oils) for controlled or sustained release.

The compositions may be packaged into vials or other suitable containers, including individual or multi-dose ampoules.

In preferred embodiments, the compositions comprise a therapeutically effective amount of the BoNT. The term “therapeutically effective amount” refers to an amount sufficient to effect beneficial or desirable cosmetic or medical results. That result can be reducing, alleviating, inhibiting, or preventing one or more symptoms of a condition. For any active agent, a therapeutically effective amount can be estimated initially in cell culture assays or in animal models, usually mice, rabbits, dogs, or pigs. An animal model may also be used to determine a desirable concentration range and route of administration.

In some embodiments, the compositions comprise a dosage of about 0.01 to 2,000 units, about 0.1 to 1,000 units, or about 0.5 to 600 units of BoNT. One unit is defined herein as an amount of the toxin with which a half of mice die within 4 days when administered intraperitoneally (1 LD50).

### Methods of Treatment:

In a sixth aspect, the present invention provides methods of treating a condition in a subject. The methods comprise administering a BoNT or composition described herein the subject to treat the condition.

“Treating” describes something that is done to a subject to combat a condition. Treating may involve controlling the condition, lessening its symptoms or complications, slowing its progression, and/or eliminating it.

As used herein, the term “condition” is used to describe both health problems (e.g., diseases, disorders) and cosmetic issues. Any condition that could potentially benefit from treatment with a BoNT-based therapy can be treated using the methods of the present invention. In some embodiments, the condition is one that is commonly treated using a BoNT, such as a pain condition, movement disorder, neurological disorder, neuromuscular disorder, spasticity, or cosmetic issue. In specific embodiments, the condition is cervical dystonia, blepharospasm, severe primary axillary hyperhidrosis, strabismus, achalasia, chronic focal neuropathies, migraine or another headache disorders, muscle spasms, upper motor neuron syndrome, sweating, hemifacial spasm, spasmodic torticollis, post-stroke spasticity, cerebral palsy, spasmodic dysphonia, lumbago, stiffness in the shoulders, paresis occurring at the onset of Parkinson disease or multiple sclerosis, myofascial pain syndrome, spasm of the masticatory muscles, chronic anal fissure, overactive bladder, bruxism, facial myokymia, a tic, local dystonia, or wrinkles.

As used herein, the term “administering” refers to the introduction of a substance into or onto a subject's body. Methods of administration are well known in the art and include, but are not limited to, oral administration, transdermal administration, administration by inhalation, nasal administration, topical administration, intravaginal administration, ophthalmic administration, intraoral administration, intracerebral administration, rectal administration, sublingual administration, buccal administration, and parenteral administration, including injectable such as intravenous administration, intra-arterial administration, intramuscular administration, intradermal administration, intrathecal administration, and subcutaneous administration. In preferred embodiments, the composition is administered topically or via injection. In specific embodiments, the composition is administered via intramuscular injection or subcutaneous injection. Administration can be performed in a single dose or multiple doses and may be continuous or intermittent. In some embodiments, the composition is administered in a therapeutically effective amount.

The “subject” to which the methods are applied may be a mammal or a non-mammalian animal, such as a bird. Suitable mammals include, but are not limited to, humans, cows, horses, sheep, pigs, goats, rabbits, dogs, cats, bats, mice, and rats. In certain embodiments, the methods may be performed on lab animals (e.g., mice and rats) for research purposes. In other embodiments, the methods are used to treat commercially important farm animals (e.g., cows, horses, pigs, rabbits, goats, sheep, and chickens) or companion animals (e.g., cats and dogs). In preferred embodiments, the subject is a human.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference unless explicitly indicated otherwise. The present disclosure shall control in the event there are any disparities between any definitions and/or descriptions found in the cited references.

The following examples are meant only to be illustrative and are not meant as limitations on the scope of the invention or of the appended claims.

### Illustrative Embodiments

1. An expression vector comprising a promoter operably linked to a polynucleotide encoding a botulinum neurotoxin (BoNT).
2. The expression vector of embodiment 1, wherein the promoter is a ferredoxin promoter, a thiolase promoter, or a tent promoter.
3. The expression vector of any one of the preceding embodiments, wherein the expression vector is a shuttle vector.
4. The expression vector of any one of the preceding embodiments, wherein the vector further comprises a selectable marker.
5. The expression vector of any one of the preceding embodiments, wherein the BoNT is type A (BoNT/A), type B (BoNT/B), type C (BoNT/C), type D (BoNT/D), type E (BoNT/E), type F (BoNT/F), or type G (BoNT/G).
6. The expression vector of any one of the preceding embodiments, wherein the BoNT is an engineered BONT.
7. An atoxic Clostridium bacterium engineered to express a heterologous BONT.
8. The bacterium of embodiment 7, wherein the bacterium is an atoxic Clostridium tetani.
9. The bacterium of embodiment 8, wherein the bacterium is:
   a) Clostridium tetani ATCC 454; or
   b) Clostridium ATCC 19406.
10. The bacterium of any one of embodiments 7-9, wherein the bacterium comprises the expression vector of any one of embodiments 1-6.
11. The bacterium of any one of embodiments 7-10, wherein the BoNT is type A (BoNT/A), type B (BoNT/B), type C (BoNT/C), type D (BoNT/D), type E (BoNT/E), type F (BoNT/F), or type G (BoNT/G).
12. The bacterium of any one of embodiments 7-11, wherein the BoNT is an engineered BONT.
13. The bacterium of any one of embodiments 7-12, wherein the bacterium produces active BONT in the absence of neurotoxin-associated proteins.
14. A method of making the bacterium of any one of embodiments 7-13, the method comprising introducing a polynucleotide encoding the BoNT into the bacterium.
15. The method of embodiment 14, wherein the polynucleotide is introduced via conjugation with a donor strain.
16. The method of embodiment 14 or 15, wherein the polynucleotide is part of an expression vector.
17. The method of embodiment 16, wherein the expression vector is the expression vector of any one of embodiments 1-6.
18. A method of producing a BoNT, the method comprising:
   a) culturing the bacterium of any one of embodiments 7-13 in a culture medium; and
   b) collecting the BoNT from the culture.
19. The method of embodiment 18, wherein the bacterium is cultured for 1-14 days prior to step (b).
20. The method of embodiment 18 or 19, wherein step (b) comprises removing the bacterium from the culture medium to produce a cell-free culture supernatant comprising the BoNT.
21. The method of embodiment 20, further comprising purifying the BoNT from the cell-free culture supernatant.
22. The method of any one of embodiments 18-21, wherein the culture medium is a modified Mueller Miller medium.
23. A BONT made by the method of any one of embodiments 18-22.
24. A composition comprising an engineered BONT.
25. A method of treating a condition in a subject comprising administering the composition of embodiment 24 to the subject to treat the condition.
26. The method of embodiment 25, wherein the condition is a pain condition, movement disorder, neurological disorder, neuromuscular disorder, spasticity, or cosmetic issue.
27. The method of embodiment 25 or 26, wherein the condition is axillary hyperhidrosis, migraine, cervical dystonia, blepharospasm, strabismus, achalasia, upper motor neuron syndrome, sweating, or wrinkles.
28. The method of any one of embodiments 25-27, wherein the composition is administered topically or via injection.
29. The method of embodiment 28, wherein the composition is administered via intramuscular injection or subcutaneous injection.
30. The method of any one of embodiments 25-29, wherein the subject is a human.

### EXAMPLES

Expression of Recombinant Clostridial Neurotoxin by C. tetani

Reference is made to Gregg, B. M.; Gupta, S.; Tepp, W. H.; Pellett, S. “Expression of Recombinant Clostridial Neurotoxin by C. tetani.” Microorganisms 2024, 12, 2611, which is incorporated by reference herein in its entirety.

Tetanus neurotoxins (TeNT) and botulinum neurotoxins (BoNTs) are closely related ~150 kDa protein toxins that together comprise the group of clostridial neurotoxins (CNTs) expressed by various species of Clostridia bacteria. While TeNT is expressed as a single polypeptide, BoNTs are always produced alongside multiple non-toxic proteins that form a stabilizing complex with the BoNT and are encoded in a conserved toxin gene cluster. It is unknown how tent evolved without a similar gene cluster and why complex-free TeNT is secreted as a stable and soluble protein by Clostridium tetani, whereas complexing proteins appear to be essential for BONT stability in culture supernatants of Clostridium botulinum. To assess whether the stability of TeNT is due to an innate property of the toxin or is a result of C. tetani's intra- and extra-cellular environment, both TeNT and complex-free BoNT/A1^{ERY} (i.e., a catalytically inactive BoNT/A1 mutant comprising the point E224Q, R363A, and Y366F) were expressed recombinantly in atoxic C. tetani and analyzed for expression and stability. The strong clostridial ferredoxin (fdx) promoter resulted in expression of recombinant TeNT at greater levels and earlier time points than endogenously produced TeNT. Recombinant BoNT/A1^{ERY} (rBoNT/A1^{ERY}) was similarly expressed by atoxic C. tetani, although partial degradation was observed. The rBoNT/A1^{ERY} produced in C. tetani was also partially proteolytically processed to the dichain form. Investigations of bacterial growth media and pH conditions found that the stability of rTeNT and rBoNT/A1^{ERY} in spent media of C. tetani or C. botulinum was affected by growth media but not by pH. These data indicate that the distinct metabolism of C. tetani or C. botulinum under various growth conditions is a primary factor in creating a favorable environment for complex-free CNT stability.

Tetanospasmin, or tetanus neurotoxin (TeNT), is a bacterial toxin expressed by the anaerobic, Gram-positive, bacilli Clostridium tetani (C. tetani). TeNT and the closely related botulinum neurotoxins (BoNTs), together known as the clostridial neurotoxin (CNT) family, are the most potent natural toxins known to humans [1]. TeNT is the agent responsible for tetanus, a potentially deadly disease causing long-lasting spastic paralysis of skeletal muscles. The dramatic clinical presentations of tetanus, including locked jaw and extreme muscle spasms, have been noted in medical manuscripts as early as the third to fifth century BC. However, it was not until the late 19th century that the cause of the disease was determined to be the TeNT produced by C. tetani [2,3]. C. tetani spores are found nearly ubiquitously across the world in soil, dirt, and manure, and may germinate and begin TeNT expression when exposed to a sufficiently anaerobic environments such as a deep wound [3,4].

TeNT is expressed by vegetative C. tetani cells as a ~150 kDa single-chain polypeptide, which the bacteria proteolytically process into a 50 kDa light chain (LC) and 100 kDa heavy chain (HC) linked by a disulfide bond [5]. If humans are exposed to the toxin through infection of a deep wound, TeNT holotoxin is released into the circulation and makes its way to the peripheral nervous system, where it is endocytosed into motor-neuron nerve endings, followed by intra-axonal retrograde transport and transcytosis to inhibitory interneurons of the central nervous system [6-8]. There, the zinc endopeptidase LC is released into the cytosol of inhibitory neurons and cleaves vesicle-associated membrane protein 2 (VAMP2). VAMP2 is a critical component in assembling soluble NSF attachment protein receptor (SNARE) complexes [9], and its cleavage halts SNARE-mediated membrane fusion of neurotransmitter-containing vesicles, thus blocking release of the inhibitory neurotransmitters gamma-aminobutyric acid (GABA) and glycine [10,11]. This results in continued neuromuscular excitation and the spastic paralysis characteristic of tetanus. TeNT is extremely potent, with an estimated minimum mouse lethal dose of only 0.2 ng/kg in humans [12], although the development of a successful toxoid vaccine has markedly lowered tetanus case numbers in countries with robust vaccination programs [4].

BoNTs are a family of closely related protein toxins and are the agents responsible for botulism, a rare but extremely serious disease characterized by descending flaccid paralysis [1,13]. BoNTs are produced by several species of Clostridium, including Clostridium botulinum (C. botulinum), and have been traditionally categorized into seven immunogenically distinct serotypes (BoNT/A-G), which contain over 45 subtype variations and mosaic toxins [14,15]. Like TeNT, BoNTs are ~150 kDa AB toxins consisting of a catalytically active LC linked via disulfide bond to a HC responsible for neuronal cell binding and LC translocation [16,17]. BoNTs similarly target vertebrate neurons, although intoxication occurs primarily at cholinergic nerve terminals, where LC-mediated cleavage of various SNARE targets prevents fusion of acetylcholine-containing vesicles to the membrane of the presynaptic nerve terminal [18,19]. Absence of acetylcholine signaling in the synaptic cleft results in flaccid muscular paralysis, as opposed to the spastic paralysis brought about by TeNT intoxication.

Although TeNT and BoNTs share a great deal of similarity regarding overall toxin structure and function, there are several key differences. BoNTs are highly diverse, with bont genes discovered across the plasmids, bacteriophages, and chromosomes of multiple Clostridial species. Across serotypes, bont toxin genes range in nucleotide identity from ~32-75% [20]. All bont serotypes and subtypes are found clustered with genes encoding neurotoxin-associated proteins (NAPs), which stabilize and protect BoNTs from chemical and enzymatic threats [1,21] and aid their passage through gastrointestinal tracts after oral exposure [22]. NAPs encoded within the gene cluster vary by toxin serotype [23], although all presently discovered serotypes possess a gene encoding the protein non-toxic non-hemagglutinin (NTNH). NTNH is an ~130-150 kDa protein sharing much of its domain structure with BoNTs, and it binds the associated BONT to form a ~300 kDa minimal progenitor complex (M-PTC) [24,25]. Binding of additional NAPs to NTNH results in a full progenitor complex (PTC) that is up to ~900 kDa in size [24].

In contrast, the tent operon is simpler, consisting only of a plasmid-encoded promoter region, the positive regulator tetR, and adjacent tent gene [26,27]. Unlike bont genes, tent is highly conserved among toxigenic C. tetani strains and no immunogenically distinct TeNT serotypes have been reported [28]. There also is no evidence of TeNT forming a complex with NAPs analogous to those of BoNTs or otherwise. This prompts the question of what allows stable production of TeNT without the need for complexing proteins, when there appears to be universal evolutionary pressure for retention of BoNT NAP genes [22]. The goal of this study was to determine if the stability of TeNT in C. tetani culture supernatants is due to the environment created by C. tetani metabolism or to resistance of TeNT to proteases that may be present in C. tetani culture supernatant.

Expression analyses of rCNTs in an atoxic C. tetani strain indicate high production levels of both rTeNT and rBoNT^{ERY} dependent on the promoter used and with at least partial proteolytic processing to the dichain. Comparative analysis of the relative stability of BoNT/A1 and TeNT in spent C. botulinum and C. tetani culture media suggested that both rCNTs were susceptible to degradation and that modified Mueller Miller media enhanced stability of rCNTs. Our data reveal that stability of complex-free TeNT and BoNT/A was similarly affected by the environment created as a result of metabolism during fermentation of C. tetani and C. botulinum and suggest that an atoxic C. tetani strain lacking the endogenous tent may be useful as a high-level production system of complex-free rBONT and rTeNT.

### Results

C. tetani 454 and 19406 are Compatible Recipient Strains for Conjugative Plasmid Transfer:

Two atoxic C. tetani strains were chosen for our analysis of rTeNT and rBoNT expression due to their unique properties and relatedness to the Harvard strain that is used industrially to produce TeNT [29,30]. Strain ATCC 19406 is a North American laboratory diagnostic strain that is closely related to the Harvard strain but that lacks the entire 74 kb toxin-encoding plasmid [31]. C. tetani ATCC 454 is an atoxic strain isolated from Chinese clinical fecal samples in the 1920s and is also closely related to the Harvard strain, although it only lacks an approximately 20 kb segment of the plasmid including both tetR and tent [31,32].

As little information is published regarding antibiotic susceptibility of C. tetani strains outside of a clinical context, the inventors first screened both strains of C. tetani as well as the potential E. coli conjugative donor strains CA434, S. Express, Interstellar, and TopSex [33,34] for susceptibility to a variety of commonly used antibiotics and antibiotic combinations at various concentrations (Table 1). Both C. tetani and E. coli are sensitive to thiamphenicol at 15 mg/mL, so the inventors opted to use plasmid vectors containing the thiamphenicol resistance gene catP to select for maintenance of the desired plasmid [35]. Additionally, the inventors chose the following antibiotic combination to select against the E. coli donor strain as it was effective to select against all tested donor strains: trimethoprim (1.5 mg/mL), sulfamethoxazole (30 mg/mL), and cycloserine (250 mg/mL) (TSC). Utilization of TSC+thiamphenicol allowed for selection of only C. tetani containing the desired plasmid after conjugation.

TABLE 1
Antibiotic sensitivity profiles of C. tetani and E. coli strains

| | C. tetani | C. tetani | E. coli | E. coli | E. coli | E. Coli |
|---|---|---|---|---|---|---|
| Antibiotic | 454 | 19406 | CA434 | S. Express | Interstellar | TopSex |
| Cycloserine | Resistant | Resistant | Weakly | Weakly | Weakly | Weakly |
| (250 μg/mL) | | | resistant | resistant | resistant | resistant |
| Kanamycin | NT | NT | Resistant | Resistant | Resistant | Resistant |
| (50 μg/mL) | | | | | | |
| Spectinomycin | Weakly | Weakly | NT | Resistant | Resistant | NT |
| (250 μg/mL) | resistant | resistant | | | | |
| Thiamphenicol | Sensitive | Sensitive | Resistant | Resistant | Resistant | Resistant |
| (15 μg/mL) | | | | | | |
| Rifampicin | Sensitive | Sensitive | NT | NT | NT | NT |
| (50 μg/mL) | | | | | | |
| Nalidixic Acid | Sensitive | Sensitive | Sensitive | NT | NT | NT |
| (90 μg/mL) | | | | | | |
| Erythromycin | Sensitive | Sensitive | NT | NT | NT | NT |
| (50 μg/mL) | | | | | | |
| Ampicillin | Sensitive | Sensitive | Sensitive | Sensitive | Sensitive | Sensitive |
| (50 μg/mL) | | | | | | |
| Tetracycline | Sensitive | Sensitive | NT | Resistant | Resistant | NT |
| (3 μg/mL) | | | | | | |
| Sulfamethoxazole | Resistant | Resistant | Resistant | Resistant | Resistant | NT |
| (30 μg/mL) | | | | | | |
| Trimethroprim | Resistant | Resistant | Resistant | Resistant | Resistant | NT |
| (1.5 μg/mL) | | | | | | |
| Sulfamethoxazole + | Resistant | Resistant | Weakly | Weakly | Weakly | NT |
| Trimethroprim (“co- | | | resistant | resistant | resistant | |
| trimoxazole”) | | | | | | |
| (30 μg/mL, 1.5 | | | | | | |
| μg/mL) | | | | | | |
| Sulfamethoxazole + | Resistant | Resistant | Sensitive | Sensitive | Sensitive | NT |
| Trimethroprim + | | | | | | |
| Cycloserine (“TSC”) | | | | | | |
| (30 μg/mL, 1.5 | | | | | | |
| μg/mL, 250 μg/mL) | | | | | | |

NT: Not tested. Weakly resistant: Number of colonies is greatly reduced (<20% compared to control plates).

To determine which origins of replication may be utilized in these strains, the inventors screened several modular pMTL8000 series clostridial shuttle vectors by conjugating them into C. tetani 19406 and 454. pMTL82151 (C. botulinum replicon pBP1) and pMTL87151 (C. perfringens replicon pIP404) [35,36] reliably yielded trans-conjugants, indicating that their origins of replications are functional in these strains. However, pMTL87151 contains an internal NdeI restriction site which would complicate cloning and therefore pMTL82151 was selected as the shuttle vector for future experiments.

Finally, three promoters were examined for expressing rTeNT. pMTL82152 encodes the ribosome binding site (RBS) and thiolase promoter (thl) of C. acetobutylicum [37], while pMTL82153 encodes the RBS and ferredoxin promoter (fdx) of C. pasteurianum [38]. pMTL82155 was created by cloning the region encoding the C. tetani positive regulator tetR, the endogenous tetR promoter (p_{tetR}), and the tent promoter (pₜₑₙₜ) from a wild-type C. tetani strain (64001) into pMTL82151 [39].

Following successful cloning and sequence verification of the tent constructs, the inventors observed that several plasmids underwent rearrangement following transformation into the E. coli donor strains. E. coli CA434, a recA+ strain, was used as the preferred donor strain, and resulted in the successful conjugation of pMTL82153-TeNT into both C. tetani 454 and 19406. The pMTL82155-TeNT was conjugated into C. tetani 454 via E. coli S. Express. The pMTL82152-TeNT plasmid was consistently rearranged in any of the tested E. coli donor strains and thus was not further pursued.

The Fdx Promoter Stimulates rTeNT Expression Earlier and in Greater Quantities than tetR and Wild-Type Strains:

Expression of rTeNT in C. tetani strain 454 and 19406 was examined in three different growth media using three different promoters. Mueller and Miller developed a media formulation for increased expression of TeNT [29], which has been analyzed and improved upon in the decades since its' creation. Of specific interest were formulations by Latham et al., (‘modified Mueller Miller media’) which removed several inorganic salts and amino acids [40], and by Demain et al., (‘HySoy media’) which replaced animal and dairy products with soy-based alternatives [41,42]. A final option, ‘HySoy-T’, is a commercially available media which utilizes an alternative source of soy peptides but is otherwise identical to the HySoy formulation. Each of these media was supplemented with 0.5 g/L of reduced iron powder which has been shown to increase toxin production in C. tetani [42,43].

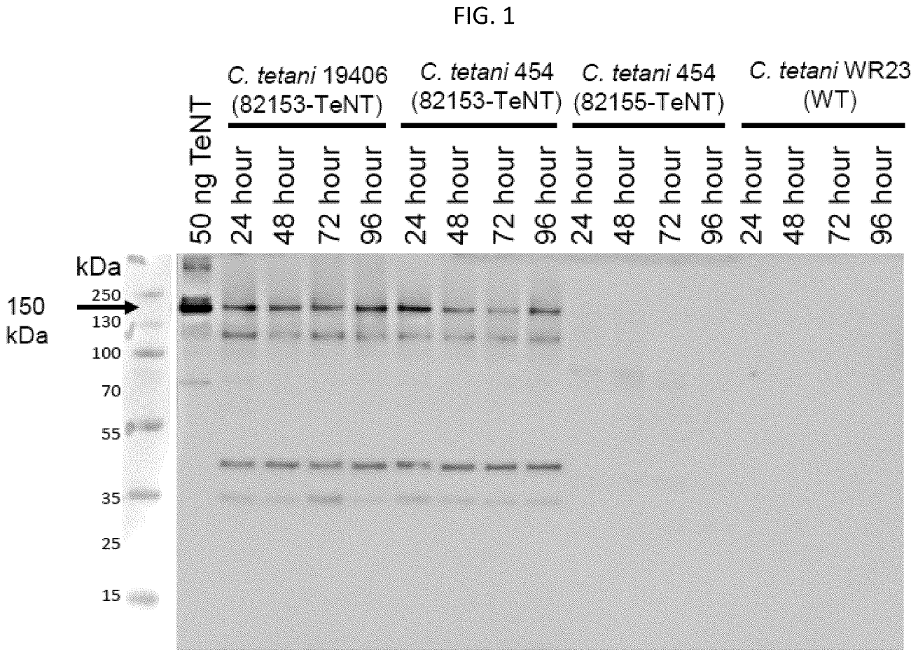

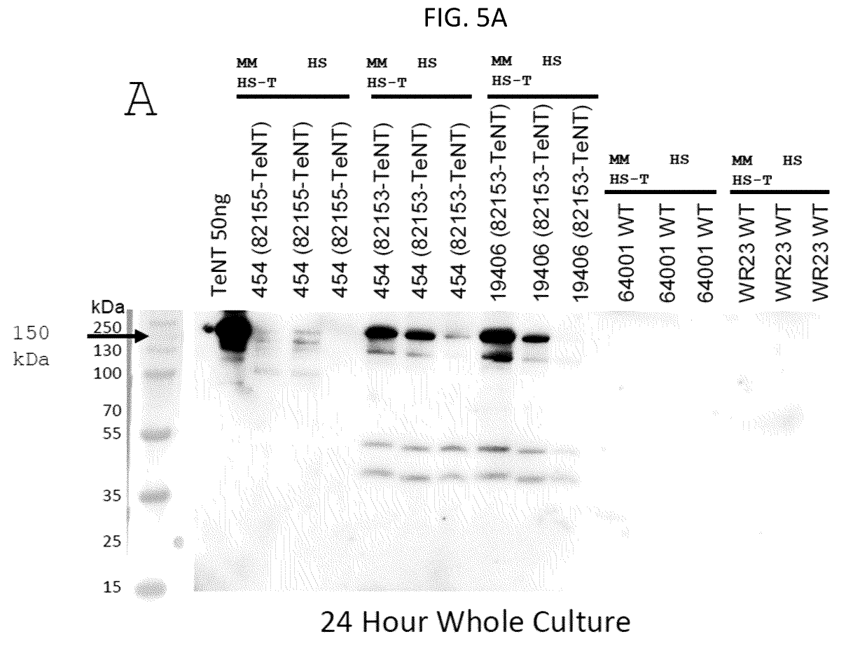

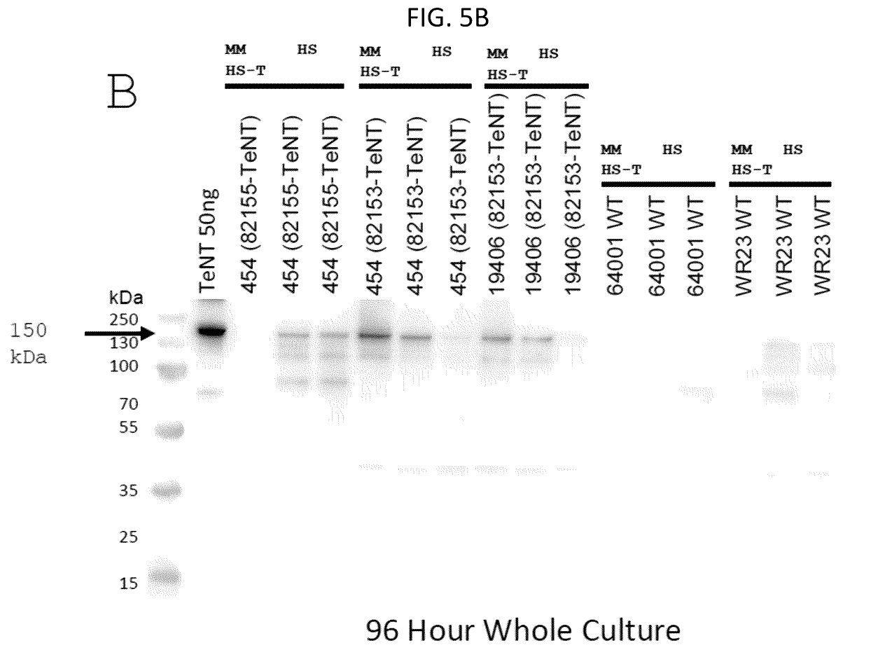

Western blot analysis of whole bacterial culture revealed that expression of ~150 kDa TeNT holotoxin was greater in C. tetani strains containing the pMTL82153-TeNT plasmid compared to the pMTL82155-TeNT plasmid or expression in wild-type C. tetani strains, although toxin varied in quantity and stability depending on culture formulation (FIG. 5). The highest and most consistent expression levels of rTeNT were observed in modified Mueller Miller (+reduced iron), where rTeNT expression in C. tetani 454/19406 (82153-TeNT) was first observed at 24 h and remained constant until 96 h of culture (FIG. 1). In contrast, endogenous TeNT production by a wild-type C. tetani and rTeNT production driven by the endogenous promoter (C. tetani 454 (82155-TeNT)) was not reliably detected in Mueller Miller media using the same western blot conditions (FIG. 1) but was observed after 96 hours of growth in HySoy or HySoy-T media (FIG. 5). This toxin production coincided with a rapid pH drop from the roughly neutral pH up to 72 hours to pH~4.8-5.2 by 96 hours. Even then, the quantity of TeNT expressed was lower than that of either 454 (82153-TeNT) or 19406 (82153-TeNT). These data indicate rTeNT expression under the control of the fdx promoter in atoxic C. tetani results in higher and more reliable production of the toxin independent of the metabolic shifts required for endogenous TeNT production [44,45]. Both C. tetani 454 and 19406 expressed rTeNT at roughly the same levels, with no discernable difference in toxin breakdown between the two. However, C. tetani strain 19406 tended to swarm after conjugation, making it difficult to isolate single colonies. Thus, strain 454 was chosen for additional experiments.

Full-Length Recombinant BoNT/AT^{ERY} is Produced by C. tetani 454 without Complexing Proteins:

To examine if other CNTs may be stably expressed in C. tetani 454, the inventors generated a pMTL expression construct encoding catalytically inactive BoNT/A1^{ERY} (pMTL82153-BoNT/A1^{ERY}) and conjugated it into C. tetani 454 via the E. coli donor strain CA434.

C. tetani 454 (82153-A1^{ERY}) was cultured alongside C. botulinum Hall A hyper/tox-(83152-A1^{ERY}), which has previously been shown to produce high levels of rBoNT/A complex [46-48]. C. botulinum Hall A hyper/tox-retains genes encoding BoNT/A NAPs, allowing comparison of BoNT/A1^{ERY} expression in Clostridial strains with and without native complexing proteins. Samples were cultured in TPGY, modified Mueller Miller media (+reduced iron), and toxin production media (TPM) for 24-96 hours. Modified Mueller Miller media is commonly used to support TeNT production in C. tetani and TPM is commonly used to express BoNTs in C. botulinum strains, and TPGY is a general clostridial growth media.

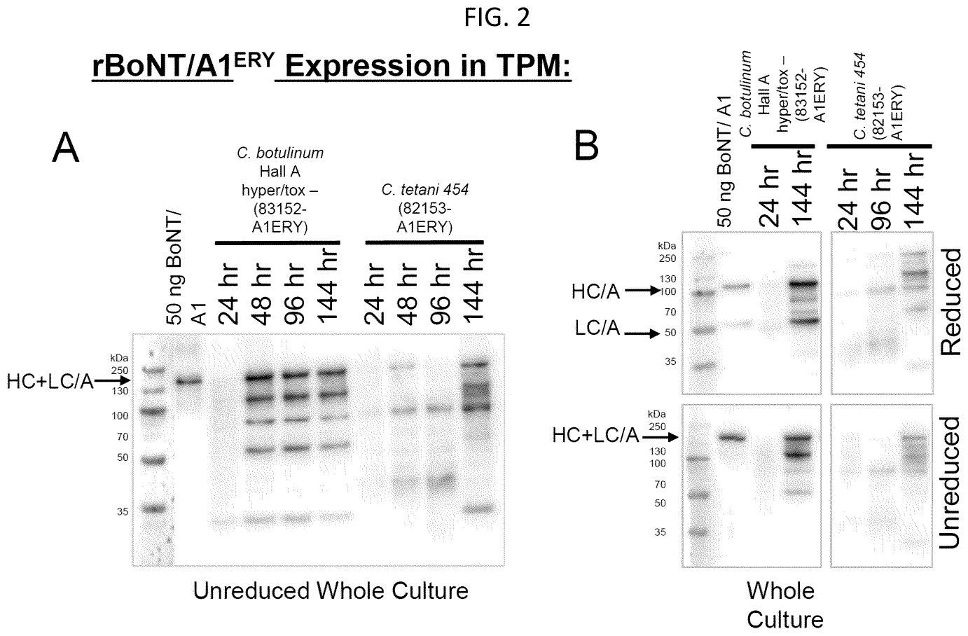

Western blot analysis demonstrated that fermentation in TPGY resulted in a small amount of 150 kDa full-length rBoNT/A1^{ERY} in both C. tetani 454 and C. botulinum Hall A hyper tox-, but many smaller bands indicating degradation were observed (data not shown), suggesting that TPGY may not be an optimal expression media. In C. tetani 454 (82153-A1^{ERY}) cultured in TPM, full-length rBoNT/A1^{ERY} was detected as early as 24 hours in the cell pellet with increasing amounts detected in whole culture samples by 144 hours (FIG. 2A). The toxin appeared to be partially post-translationally processed into the active dichain form at 144 hours, as evidenced by the separation of the ~150 kDa holotoxin into ~100 kDa heavy chain and ~50 kDa light chain after reduction of the sample by DTT (FIG. 2B). This demonstrates that a recombinant complex-free botulinum toxin can be expressed by a C. tetani strain and can be converted to its activated dichain form via endogenous proteases. However, compared to production of rBoNT/A1^{ERY} complex by C. botulinum Hall A hyper tox-, production in C. tetani grown in TPM was lower and occurred later during culture (FIG. 2). In both cultures, a significant portion of the toxin appeared degraded as evidenced by smaller molecular weight bands on the western blot (FIG. 2).

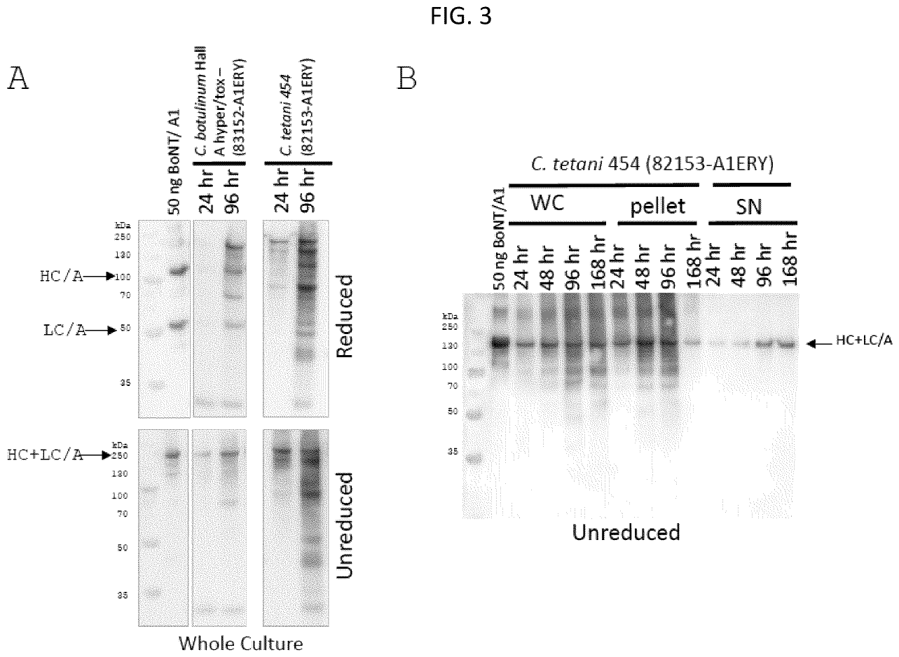

Similarly, when cultured in modified Mueller Miller (+reduced iron) media, the full-length toxin expressed by C. tetani 454 (82153-A1^{ERY}) appeared more degraded than that of rBoNT/A1^{ERY} expressed in C. botulinum Hall A hyper/tox-(FIG. 3A). Prior studies have reported that the mechanism by which reduced iron powder increases C. tetani growth and toxin production may be providing an insoluble surface area [49]. Examination of rBoNT/A1^{ERY} expression by C. tetani 454 (82153-A1^{ERY}) on modified Mueller Miller media agar plates without the addition of reduced iron resulted in strong toxin production with minimal break-down (FIG. 3B), supporting the idea that the presence of an insoluble growth surface plays a role in C. tetani growth and toxin production.

### The Role of pH in CNT Expression and Stability:

A previous study indicated that a culture pH below pH 6 plays a critical role in BoNT complex stability produced by C. botulinum [47]. In this study, culture pH varied depending on growth conditions (Table 2). C. tetani 454 (82153-TeNT) cultured in Mueller Miller (+reduced iron) had an average (n=3) culture media pH of ~5.2 at 24 hours and ~4.9 by 144 hours, with TeNT production detected throughout the time period (FIG. 1). When the same sample was cultured in TPM, the pH was similar, averaging~5 at 24 hours and ~4.9 at 144 hours. However, C. tetani 454 (82153-A1^{ERY}) cultured in modified Mueller Miller (+reduced iron) had an average pH of ~6.7 at 24 hours which dropped to ~6.2 by 144 hours, and when cultured in TPM the samples maintained a pH of ~6.9-7.0 from 24-144 hours. This trend was very similar for the negative, toxin-free expression control C. tetani 454 (82153), indicating that the tent gene cluster itself might contribute to the metabolically induced pH drop of endogenous C. tetani [39,44,45].

TABLE 2
Average pH of culture media over time

| | pH 24 Hour | pH 48 Hour | pH 96 Hour | pH 144 Hour |
|---|---|---|---|---|
| C. tetani 454 (82153-TeNT)- | 5.17 | 5.25 | 5.38 | 4.89 |
| Cultured in MM (+ iron) | | | | |
| C. tetani 454 (82153-TeNT)- | 4.95 | 4.78 | 5.00 | 4.86 |
| Cultured in TPM | | | | |
| C. tetani 454 (82153-A1ERY). 1- | 6.71 | 6.50 | 6.18 | 6.15 |
| Cultured in MM (+ iron) | | | | |
| C. tetani 454 (82153-A1ERY)_ | 6.93 | 7.17 | 7.23 | 7.05 |
| Cultured in TPM | | | | |
| C. tetani 454 (82153) - | 6.80 | 7.50 | 7.33 | 7.18 |
| Cultured in MM (+ iron) | | | | |
| C. tetani 454 (82153) - | 6.97 | 7.12 | 7.19 | 7.24 |
| Cultured in TPM | | | | |
| Wild-type C. tetani WR23- | 6.84 | 7.50 | 6.37 | 6.50 |
| Cultured in MM (+iron) | | | | |
| Wild-type C. tetani WR23- | 6.87 | 7.10 | 7.18 | 7.16 |
| Cultured in TPM | | | | |

Average pH of n = 3 replicate hungate culture tubes.
MM: modified Mueller Miller media + 0.5 g/L reduced iron powder.
TPM: toxin production media.

BoNT/A1 Holotoxin is More Stable in Spent Modified Mueller Miller Media than in Spent TPM:

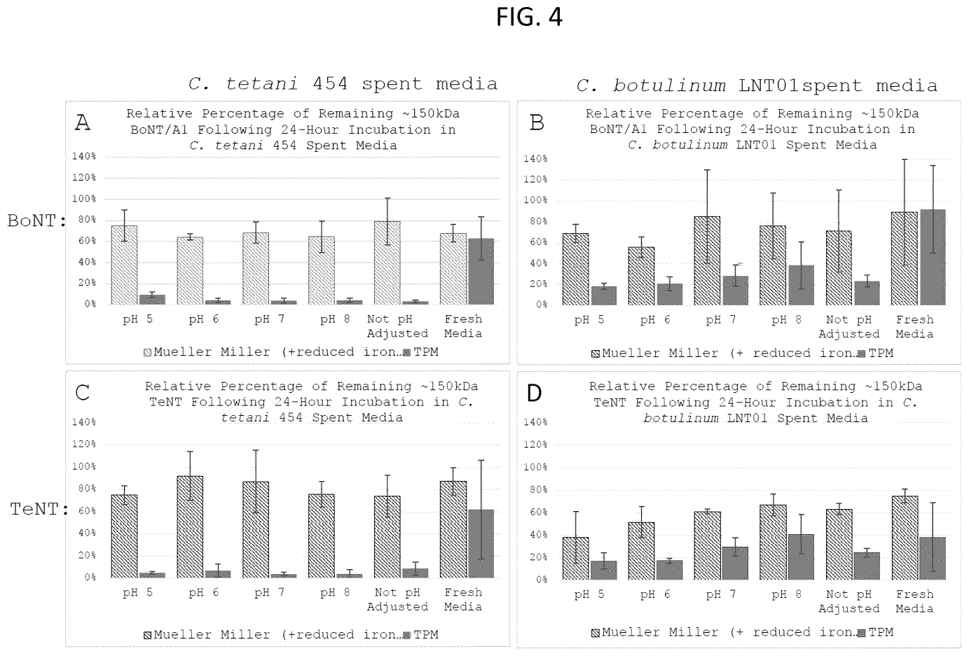

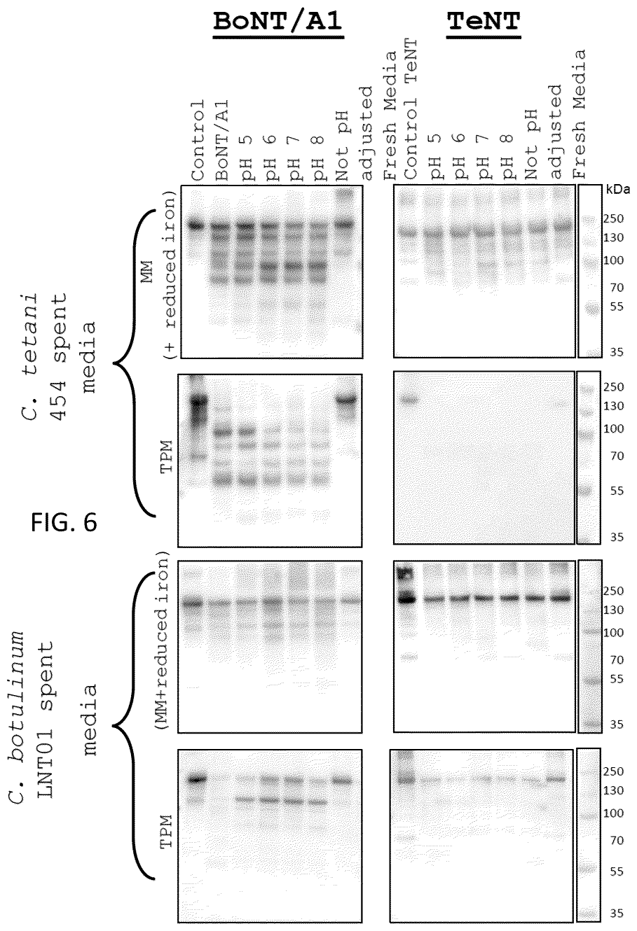

To directly examine pH dependence of complex-free BoNT/A1 and TeNT stability in culture supernatants, the rCNTs were incubated in spent media of the negative expression control C. tetani 454 (82153) as well as C. botulinum LNT01 cultured in modified Mueller Miller (+reduced iron) or TPM for 4 days. Unlike C. botulinum Hall A hyper/tox-, C. botulinum LNT01 lacks the entire bont gene cluster including all complexing protein genes [50]. This allowed examination of the effects of culture pH on BoNT/A and TeNT without the possibility of any stabilizing effect from type-A complexing proteins. After 24-hour incubation of BoNT/A1 or TeNT in sterile filtered spent culture media adjusted to pH 5, 6, 7, or 8, remaining 150 kDa BoNT/A1 and TeNT was assessed by western blot (FIG. 6) and densitometry (n=3), and in all cases, the pH of spent media had little or no impact on CNT stability (FIG. 4). However, a large difference was noticed between the two tested media formulations. Spent TPM of C. tetani 454 resulted in almost no detectable 150 kDa toxin, but primarily smaller protein bands consistent with specific cleavage of the protein toxin (FIG. 6). Less protein breakdown and more 150 kDa protein was observed in spent TPM of C. botulinum LNT01. However, even in that case, the amount of 150 kDa TeNT or BoNT was reduced to 20-40% with distinct break-down products observed for BoNT/A1 but not for TENT (FIG. 6). Both BoNT/A1 and TeNT exhibited far less degradation in modified Mueller Miller media than in TPM with between 60-80% of the added protein amount remaining as the 150 kDa band (FIG. 4), although few distinct break-down products were still clearly visible (FIG. 6). Interestingly, some break-down of the 150 kDa protein was observed even in fresh media for both BoNT/A1 and TeNT, although to a much lesser extent than in the spent media, indicating that some degradation is potentially due to auto-proteolysis, aggregation, or an SDS-PAGE gel related artifact.

### Discussion

Of all the clostridial neurotoxins, TeNT is unique in that it is not encoded in a gene cluster alongside non-toxic neurotoxin associated proteins (NAPs) and doesn't require NAPs to be produced as a stable protein in fermentation cultures [1]. Free BoNTs (without NAPs), on the other hand, are readily degraded by environmental and endogenous proteases [21,51], whereas complexing with NAPs to form large, multi-protein complexes prevents protein breakdown under certain conditions and increases BONT oral toxicity [24,39]. Production of TeNT by C. tetani is tightly controlled by a complex network of regulatory genes [39,44,45] and has been optimized for large scale production of stable and soluble protein to be processed to the widely used inactivated tetanus protein vaccine [29,53]. The most commonly used production media for TENT is Modified Mueller Miller [40].

Given the structural and functional similarities between BONT and TeNT holotoxins, the inventors hypothesized the intra and extracellular conditions of C. tetani may be responsible for toxin stabilization and would support recombinant expression of BoNTs without their native complexing proteins. To the inventors' knowledge, the disclosed data demonstrate the first recombinant expression of a full-length and proteolytically activated rBoNT by a C. tetani strain.

The inventors investigated expression of rTeNT and rBoNT/A1^{ERY} production in C. tetani using the modular pMTL8000 expression vector system combined with two C. tetani strains that are closely related to the Harvard strain used for large scale production of TeNT [53]. Promoters for thiolase (thl; resulting in pMTL82152) and ferredoxin (fdx; resulting in pMTL82153), which are often used for high level gene expression in Clostridial species [45], as well as native positive regulator of tent, tetR (resulting in pMTL82155), were examined [26,27]. Despite successful cloning and plasmid sequence verification, certain pMTL expression constructs appeared to be incompatible for transformation into E. coli donor strains. Specifically, pMTL82152-TeNT, pMTL82152-A1^{ERY}, and pMTL82155-A1^{ERY} displayed greatly increased rates of plasmid rearrangement following transformation into conjugative E. coli donor strains CA434 or S. Express. Whole plasmid sequencing of the plasmids isolated from E. coli revealed full or partial loss of the toxin gene and maintenance of the antibiotic resistance gene. In some cases, this gene loss and rearrangement occurred less than five hours post-transformation (data not shown). Ingle et al discuss similar instances of plasmid instability of plasmids containing genes under fdx and thl promoters and propose that high expression of certain genes downstream of strong constitutive promoters is likely detrimental to E. coli. The repeated loss of rent genes from plasmids pMTL82152-TeNT, pMTL82152-A1^{ERY}, and pMTL82155-A1^{ERY} after transformation into E. coli donor strains indicates potential selection pressure against these toxin genes in E. coli.

C. tetani 454/19406 (82153-TeNT) expressed rTeNT at higher quantities and with fewer breakdown products than either the wild-type strains or C. tetani 454 (82155-TeNT), which contains the endogenous tent promoter and regulator region, tetR (FIG. 1, FIG. 5). In addition, no metabolic shift [39,44,45,55] was required for high level production of rTeNT under the fdx promoter. This was the case in all three culture media, namely modified Mueller Miller (+reduced iron) [29], HySoy, and HySoy-T-, although modified Mueller Miller media consistently resulted in the highest expression levels (FIG. 1). A previous study found that addition of reduced iron powder to modified Mueller Miller culture media resulted in ~6 times greater production of TeNT compared to media lacking reduced iron [43].

Interestingly, native TeNT expressed by wild-type strains and rTeNT expressed by C. tetani under the control of the endogenous tent promoter region including tetR (82155-TeNT) was largely undetected in whole culture until ~72 and 96 hours, coinciding with a culture pH drop from neutral to ~4.8 (FIG. 5, Table 2). In C. tetani Harvard strain E88, Licona et. al reported that tetR and tetX expression is extremely low until the cell shifts from consuming free amino acids to peptides between 10-40 hours, and that the upregulation in toxin synthesis genes corresponded with an increase in culture pH to ~7.2 [55]. Other studies also observed an increase in culture pH prior to cell lysis and release of TeNT into the supernatant [56,57]. It is unclear whether the pH decrease the inventors observed is due to growth in static cultures, as opposed to an agitated bioreactor, or whether this is a product of the recombinant protein expression. C. tetani 454/19406 (82153-TeNT) samples, which expressed tent under the control of the ferrodoxin promoter, contained high levels of rTeNT in the whole culture by 24 hours, with a culture pH of 4.9-5.3 maintained throughout the duration of the expression studies (Table 2). The temporary difference in both culture pH and TeNT production between C. tetani 454 (82155-TeNT) and 454 (82153-TeNT), where the only difference is the promoter region, indicates that the tetR promoter/regulator region or the expressed TeNT itself may contribute to the metabolism of C. tetani, prompting a pH shift to acidic levels at the same time that tent is upregulated [45]. This hypothesis is consistent with C. tetani 454 (82155-TeNT) and the wild-type strain WR23 both displaying similar TeNT expression timelines and pH changes to culture media, and the C. tetani 454 (82153) control strain not displaying any pH shift (Table 2), and it raises the interesting question of a more global metabolic role for the TeNT or, more likely, TetR, beyond regulation of TeNT production.

To examine production of NAP-free rBoNT in the C. tetani expression system, the catalytically inactive bont/a JERY was cloned into the pMTL82153 under control of the ferredoxin promoter (pMTL82153-A1^{ERY}), and rBoNT/A1^{ERY} expression in C. tetani 454 (82153-A1^{ERY}) was analyzed in comparison to production of BoNT/A1^{ERY} complex in C. botulinum Hall A hyper/tox− (83152-A1^{ERY} or both C. tetani and C. botulinum). NAP-free BoNT/A1^{ERY} was produced in C. tetani 454 at about 50% reduced (TPM) or similar (MM+reduced iron) levels as BONT/A1^{ERY} complex in C. botulinum Hall A hyper/tox−, with evidence of at least partial proteolytic nicking to the dichain form that increased with increased culture time (FIG. 2, FIG. 3A). However, an increased amount of smaller protein bands, indicating higher rates of protein degradation, were observed for the NAP-free BoNT/A1^{ERY} produced in C. tetani 454 than for BONT/A1^{ERY} complex produced in C. botulinum Hall A hyper/tox− in both media. The break-down products for BoNT/A1^{ERY} produced in C. tetani versus C. botulinum varied, indicating either the presence of distinct proteases in the spent media of the two organisms, or the availability of distinct protease cleavage sites in complexed versus free BoNT/A1. Direct examination of the stability of BoNT/A1 and TeNT holotoxins (150 kDa, complex free) in spent culture media from a C. botulinum strain LNT01, which does not express any complexing proteins, and C. tetani 454 indicated similar stability in spent media from both strains, but a strong media type dependence (FIG. 4). Both toxins showed only mild partial degradation in spent Mueller Miller but strong degradation in spent TPM (FIG. 4, FIG. 6). No significant pH dependence for degradation was observed under any condition. This is in contrast to a previous study that showed strong pH dependent proteolytic degradation of BoNT/A1 complex in spent media of C. botulinum strain Hall A hyper [47], emphasizing the protective role of NAPs and the difference in conditions required for production of complex free versus complexed rBoNTs.

The unique pattern of degradation bands observed for BONT and TeNT in spent TPM and Mueller Miller media (FIG. 6) indicate differential production or secretion of rCNT degrading proteases induced by the growth media in both C. tetani and C. botulinum and emphasizes the role of nutrient induced bacterial metabolism on toxin production by affecting toxin stability post-translation. The unexpected observation of fewer break-down bands for both rTeNT and rBoNT/A1^{ERY} in spent MM media of C. botulinum LNT01 than C. tetani 454 (FIG. 6) suggests that either C. botulinum strain LNT01 produces less CNT specific protease activity, or that degradation is more complete to where fewer bands are detected on the gel. The fact that there was no significant difference in the amounts of remaining 150 kDa rCNT bands (FIG. 4) supports the latter. While these studies indicate that complex-free TeNT and BONT stability is largely independent of pH (FIG. 4), a correlation of acidic pH and greater toxin production was observed in culture (FIG. 2, FIG. 3), indicating that metabolic changes associated with pH changes may result in production of specific sets of proteases.

Overall, the relatively good stability of 150 kDa rBoNT/A1 ^{ERY} in spent Mueller Miller media of either C. tetani or C. botulinum (on average 60-80%) (FIG. 3A) also suggests that C. tetani strain 454 as well as other atoxic clostridial strains grown in specific optimized media may be utilized as a new expression system for production of complex-free recombinant BoNTs. As such an expression system is not dependent on specific NAPs for protein of the rBoNT, the system will likely be applicable for any BoNT serotypes and subtypes.

Some BoNTs are not easily purified from their native C. botulinum strains in quantities sufficient for structural and functional analysis due to low expression levels or due to being produced by a dual toxin producing strain. In addition, mutational studies require recombinant BONT production. However, recombinant BONT expression in an endogenous expression host is an invaluable, but underdeveloped, tool for further functional characterization of BoNTs. Current methods for rBoNT expression rely either on E. coli, which lacks the ability to proteolytically process and activate the toxin [58], or atoxic C. botulinum type A strains, which retain native type A NAPs and have only successfully produced BoNT/A serotypes [48]. In a recent study, the inventors found significant differences in potency of rBoNTs produced in heterologous versus endogenous expression systems [59], although the reasons underlying these potency differences are currently unknown. This study adds a novel recombinant expression system for rBoNTs utilizing an atoxic C. tetani or C. botulinum strain.

In summary, this study demonstrates that the stable production of NAP-free TeNT by C. tetani is primarily controlled by expression strain properties and metabolic pathways that can be regulated by media formulation, and that NAP-free CNT stability in growth media is independent of pH. This study also indicates that atoxic C. tetani or other atoxic clostridia can be utilized as a novel clostridial expression system for production of NAP-free rBoNTs and TeNT. The initial optimizations performed in this study to select the expression vector, promoter, atoxic C. tetani strain, and media formulations for highest production levels of rCNTs lay the groundwork for this novel expression system, and additional strain specific media and growth condition optimizations have the potential to further improve this system as a universal NAP-free CNT expression system.

### Materials and Methods

### Biosafety and Security:

The inventors' laboratory and personnel are registered with the CDC Select Agent Program for research involving botulinum neurotoxins and botulinum neurotoxin-producing strains of clostridia. The research program, procedures, occupational health plan, documentation, security, and facilities are closely monitored. All personnel have undergone suitability assessments and completed rigorous and continuing biosafety training, including biosafety level 3 (BSL3) and select agent practices before participating in laboratory studies involving botulinum neurotoxins and neurotoxigenic C. botulinum strains. All recombinant DNA protocols for the construction of the recombinant BONT genes and their expression in C. botulinum strains have been approved by the Institutional Biosafety Committee (IBC), and specific experiments were approved by the Division of Select Agents and Toxins at the CDC. A dual use research of concern (DURC) risk mitigation plan has been established and approved for these experiments.

### Media and Reagents:

Culture media reagents were purchased from Sigma Aldrich (St. Louis, MO) and BD Difco (Franklin Lakes, NJ). HySoy (PHBT30) and HySoyT (PHBT31) were purchased from Kerry (Formerly QuestSheffield; Tralee, Ireland). Antibiotics (chloramphenicol, cycloserine, kanamycin, thiamphenicol) were purchased from Sigma Aldrich and used at the following concentrations: in E. coli: kanamycin 30 mg/mL, chloramphenicol 25 mg/mL in agar and 12.5 mg/mL in liquid media. C. botulinum: thiamphenicol 15 mg/mL; trimethoprim (1.5 mg/mL), sulfamethoxazole (30 mg/mL), and cycloserine (250 mg/mL).

### Bacterial Strains:

ATCC C. tetani 454: BioProject PRJNA158341, Gen Bank assembly GCA_000987095.1; ATCC C. tetani 19406: BioProject PRJNA260197, GenBank assembly GCA_000762305.1; C. botulinum LNT01 is an atoxic strain, lacking the entire bont gene cluster including both the toxin and all associated complexing proteins.

E. coli conjugative donor strains were purchased from Plasmid Vectors (University of Nottingham, UK). Briefly, these are derivatives of commercial strains wherein the conjugative plasmid R702 has been inserted to aid in transfer of the plasmid to Clostridial species. The resulting strains, along with the commercial strain they were derived from are as follows: E. coli CA434 (HB101), S. Express (NEB, Express). Further information regarding these strains, including genotypes, may be found at plasmidvectors.com/donor-strains/. E. coli Ob was purchased from New England Biolabs (Ipswich, MA).

E. coli was grown at 37 °C in either liquid LB agitated at 225 rpm or on LB agar plates supplemented with corresponding antibiotics for plasmid selection. Clostridia cultures were grown in static nitrogen-flushed hungate tubes, and handling of culture was carried out within an anaerobic chamber (Forma Anerobic System, Marietta, OH. Atmosphere consists of 80% N₂, 10% CO₂, and 10% H₂. Cultures were grown in the indicated liquid media or agar plate formulated as follows. Tryptone peptone glucose yeast media (TPGY): 5% Trypticase peptone, 0.5% Bacto peptone, 0.4% glucose, 2% yeast extract, 0.1% L-cysteine HCl (pH 7.3-7.4); Toxin production media (TPM): 2% Trypticase peptone, 0.5% glucose, 1% yeast extract (pH 7.3); Tryptone, Yeast, Glucose media (TYG): 3% Bacto Tryptone, 2% yeast extract, 1% Glucose, 0.1% sodium thioglycolate; HySoy: 5% HySoy (Kerry, Tralee, Ireland), 0.75% glucose, 0.0125% L-cysteine, 0.0125% L-tyrosine, 0.006% FeSO₄, 0.5% NaCl, 0.05% Na₂HPO₄, 0.0175% KH₂PO₄, 0.05% MgSO₄ 7H₂O (pH 6.8). Modified Mueller Miller media was prepared as described in Lantham, et al [40]. When noted, media were prepared with reduced iron powder at a concentration of 0.5 g/L, which replaced the use of FeSO₄. 0.5 mg/mL resazurin was used as an anerobic indicator in agar plates.

Modified Mueller/Miller (Lantham et al)

| Glucose | 9.7 g/L |
|---|---|
| NZ-TT | 43.5 g/L |
| NaCl | 2.5 g/L |
| MgSO₄ | 0.1 g/L |
| Vitamin B12 | 0.05 mg/L |
| Nicotinic Acid | 0.25 mg/L |
| L-Cysteine | 125 mg/L |
| Biotin | 2.5 ug/L |
| Thiamine HC1 | 0.25 mg/L |
| Riboflavin | 0.25 mg/L |
| Pyridoxin HC1 | 0.25 mg/L |
| Calcium Pantotenate | 1 mg/L |
| Uracil | 1.25 mg/L |
| Ferrous Sulfate FeSO₄ | 60 mg/L |
| Modified Mueller/Miller with reduced iron | |
| Glucose | 9.7 g/L |
| NZ-TT | 43.5 g/L |
| NaCl | 2.5 g/L |
| MgSO₄ | 0.1 g/L |
| Vitamin B12 | 0.05 mg/L |
| Nicotinic Acid | 0.25 mg/L |
| L-Cysteine | 125 mg/L |
| Biotin | 2.5 ug/L |
| Thiamine HC1 | 0.25 mg/L |
| Riboflavin | 0.25 mg/L |
| Pyridoxin HC1 | 0.25 mg/L |
| Calcium Pantotenate | 1 mg/L |
| Uracil | 1.25 mg/L |
| Reduced iron powder | 0.5 g/L |

Antibiotic Sensitivity of C. tetani and E. coli Strains:

Triplicate samples of each strain were grown on anaerobic TPGY (C. tetani) or aerobic LB (E. coli) agar plates at 37 °C for 72 hours. Strains were considered weakly resistant if there was minimal bacterial growth compared to resistant strains, or if growth did not appear within 24-48 hours after plating.

### Generation of TeNT Plasmid Constructs:

Isolation of genomic DNA (gDNA) from wild-type C. tetani 64001: Unless otherwise stated, all centrifugation steps in gDNA isolation were performed at 16000×g. C. tetani 64001 was grown 7 hours in TPGY liquid media, a 1 mL aliquot was centrifuged for 5 minutes, and the cell pellet was resuspended in 300 mL 1×TES buffer (25 mM Tris-HCl, pH 8.0, 0.1 M NaCl, and 0.001 M EDTA), 10 mL RNase A (100 mg/mL, Qiagen) and 100 mL lysozyme (20 mg/mL, Sigma Aldrich). Following a 30-minute incubation at 37 °C, samples were treated with 50 mL 10% sodium dodecyl sulfate (SDS, ThermoFisher) and 50 mL Proteinase K (10 mg/mL, Qiagen), followed by another 30-minute incubation at 25 °C. 100 mL 5M sodium perchlorate was added, and the sample incubated 10 minutes at 25 °C. A 600 mL phenol-chloroform-isoamyl (25:24:1) mixture was dispensed into a Phasemaker tube (Invitrogen, A33248) along with 600 mL of the culture lysate sample and hand inverted 5 minutes before a 5 minute centrifugation. Supernatant was carefully transferred into 600 mL fresh phenol-chloroform-isoamyl (25:24:1) before inversion and centrifugation steps were repeated. The resulting supernatant was transferred into pre-chilled 100% EtOH and inverted until gDNA precipitation was visible and could be removed for overnight incubation in 4 °C 70% EtOH. The sample was briefly centrifuged, EtOH aspirated off, and any residual EtOH allowed to evaporate before resuspension of the gDNA in 10 mM Tris buffer, pH 8.0.

Primers ‘NdeI-tent’ and ‘tent-NheI’ were purchased from Integrated DNA Technologies Inc. (Coralville, IA), and used to amplify the tent gene from the gDNA. They include the addition of a restriction sites NdeI directly upstream and NheI directly downstream of tent to aid insertion of the gene fragment into cloning vectors.

NdeI-tent
(5′CGCATATGATACGTATGCCAATAACCATAAATAATTTTAGATATAG
TG3′, SEQ ID NO: 1)
tent-NheI
(5′CGGCTAGCTTAATCATTTGTCCATCCTTCATCTGTAGG3′, SEQ
ID NO: 2)

PCR amplification was performed using Q5 High Fidelity PCR master mix (NEB), 10 mM primers, and ~100 ng C. tetani 64001 gDNA in a GeneAmp PCR 9700 (Applied Biosystems, Foster City, CA). The resulting ~4 kb PCR-product was eluted from agarose gel using the Monarch DNA Gel Extraction Kit (NEB) and confirmed via Sanger sequencing. tent was digested with restriction enzymes NdeI and NheI to facilitate insertion into PJET1.2 (ThermoFisher) cloning vector containing either the thl or fdx promoter. All cloning steps were carried out using the CloneJet PCR Cloning Kit (K1231, ThermoFisher) according to manufacturer instruction. Resulting PJET vectors were digested with SbfI and NheI to excise thl-tent or fdx-tent regions, which were inserted into pMTL82151. This generated pMTL82152-TeNT and pMTL82153-TeNT.

tetR is located slightly upstream of tent on the E88 plasmid and is preceded by its own promoter (p_{tetR}) and followed by the tent promoter (pₜₑₙₜ) [26,39]. The inventors synthesized a segment~200 bp prior to tetR to the start of tent, flanked by Sbf1 and NdeI restriction enzyme sites for seamless insertion of the region into the shuttle vector. tetR contains an internal NdeI restriction site (CATATG), so a silent point mutation was introduced to eliminate the site (CACATG). The ~1 kb region (referred to as tetR) was synthesized by GeneArt (ThermoFisher Scientific), using the publicly available C. tetani E88 plasmid pE88 sequence (GenBank Accession AF528097.1). tetR was cloned directly into pMTL82151 using SbfI and NdeI restriction enzymes, and subsequent cloning inserted tent using NdeI and NheI restriction enzymes. This generated pMTL82155-TeNT.

### Generation of BoNT/A1^{ERY} Plasmid Constructs:

The gene for catalytically inactive BoNT/A1 (bont/a1^{ERY}) possesses three point mutations: E224Q, R363A, and Y366F. This toxin has not been shown to induce detectable toxicity [21,60], and thus is excluded from Tier 1 Select Agent regulations regarding expression of rBoNTs. bont/a1^{ERY} was excised out of PJET 1.2-bont/a1^{ERY} via restriction enzymes NheI and NdeI. This digest excised bont/a1^{ERY}, which was inserted into pMTL vectors already containing the fdx promoter region. This generated pMTL82153-BoNT/A1^{ERY}. All constructs were confirmed via whole plasmid sequencing.

Conjugation of pMTL Plasmids into C. tetani:

Plasmids were transformed into E. coli donor strains CA434 (for BoNT/A1^{ERY} constructs) or S. Express (for TENT constructs), as previously described, in LB media supplemented with 30 mg/mL kanamycin (maintenance of the R702 conjugative plasmid) and 15 mg/mL chloramphenicol (analogous to thiamphenicol; used in E. coli to maintain pMTL vectors). Plasmid constructs which displayed increased instability were prescreened prior to mating via plasmid isolation (QIA Spin MiniPrep Kit, Qiagen) followed by restriction enzyme digest. Plasmid conjugation was performed essentially as described in previous studies [34,61], with the following alterations. C. tetani 454 and 19406 were anaerobically cultured at 37 °C in un-supplemented tryptone peptone glucose yeast (TPGY) media to mid-log phase of OD600 0.6-0.7, as overgrowth of the recipient strain resulted in a decrease of overall conjugation efficiency. Liquid cultures of the transformed E. coli donor strains were then anaerobically co-incubated with liquid culture of either C. tetani 454 or 19406 on unsupplemented TPGY agar plates. Samples were allowed to mate for 24 hours at 37 °C before cultures were resuspended and transferred to fresh TPGY plates (3% agar) supplemented with the TSC+thiamphenicol antibiotic combination. After growth was visible on the plates, random colonies were selected, cultured in TPGY, and their plasmids sequenced to confirm maintenance of the toxin gene.

### Sequencing:

Whole plasmid sequencing was performed using the Oxford Nanopore MinION and Rapid Barcoding Kit 24 (SQK-RBK114.24, Oxford Nanopore, Oxford, UK) according to manufacturer instructions. Read assemblies were performed in EPI2ME, and sequences analyzed in UGENE and Mac Vector software. Sanger sequencing was performed.

### SDS-PAGE and Western Blot:

Purified inactive 8MTT TeNT was used as the TeNT toxin control, and purified BoNT/A1 holotoxin was used as the BoNT toxin control. After culturing, whole culture, pellet, and supernatant samples were collected at the indicated time points. Whole cultures were centrifuged at 16,000×g for 10 minutes to separate the cell pellet and supernatant. Cell pellets were resuspended in an equal volume of 1×TES buffer with 2 mg/mL lysozyme (Sigma Aldrich) and incubated at 35 °C for 10 minutes. For collection of cell growth from solid agar plates, each plate was flooded with 10 mL 1×PBS and manually resuspended with a plate scraper. This whole culture was then processed into supernatant and pellet samples as above. Expression samples were mixed with 4× NuPAGE LDS sample buffer (Invitrogen) to 1× with or without addition of 100 mM dithiothreitol (DTT) as a reducing agent. Samples were heated to 95 °C for 5 minutes and run on 4-12% Bis-Tris (Invitrogen) gels before transfer to 0.45 mm PVDF membrane. Western blots were probed with primary mouse polyclonal anti-8MTT (prepared in the lab) or rabbit polyclonal anti-BoNT/A1 antibody (prepared in the lab) at 1:1000 dilution. Secondary goat anti-mouse and bovine anti-rabbit alkaline phosphatase antibodies (Santa Cruz) were used at 1:7500 dilutions. Blots were exposed to KPL PhosphoGlo AP substrate (SeraCare) prior to imaging.

### Toxin Stability in Spent Culture Media:

Toxin stability experiments were conducted as described previously [47]. Briefly, 10 mL nitrogen-flushed hungate tubes of the specified media were inoculated with 100 mL of either C. tetani 454 (82153) or C. botulinum LNT01 [50]. LNT01 is an atoxic strain, lacking bont and associated complexing genes. Replicates (n=3) were cultured statically at 37 °C for 96 hours, after which the whole culture was centrifuged at 6,000×g for 10 minutes, and culture supernatants were pH adjusted to pH 5, 6, 7, and 8 via 10 N NaOH and 1 N HCl. Each replicate group additionally included a spent media sample which was not pH adjusted, and one sample of fresh media which had never supported bacterial growth. All samples were sterile filtered through 0.22 mm polyvinylidene difluoride (PVDF) filters. 50 ng (for BoNT/A1) or 250 ng (for TENT-8MTT) of sterile, purified toxin were added to each sample, and left to incubate at 37 °C for 24 hours. Samples were analyzed via SDS-PAGE and western blotting, and the concentration of the ~150 kDa protein bands were quantified via densitometry (Azure Spot).

### REFERENCES

1. Schiavo, G.; Matteoli, M.; Montecucco, C. Neurotoxins affecting neuroexocytosis. Physiol Rev 2000, 80, 717-766, doi: 10.1152/physrev.2000.80.2.717.
2. Chalian, W. AN ESSAY ON THE HISTORY OF LOCKJAW. Bulletin of the History of Medicine 1940, 8, 171-201.
3. Tejpratap S. P. Tiwari, M. P. L. M., MD, MPH; and Anna M. Acosta, MD. Epidemiology and Prevention of Vaccine-Preventable Diseases-Chapter 21. Centers for Disease Control and Prevention 2021, 14th Edition 315-328.
4. Li, J.; Liu, Z.; Yu, C.; Tan, K.; Gui, S.; Zhang, S.; Shen, Y. Global epidemiology and burden of tetanus from 1990 to 2019: A systematic analysis for the Global Burden of Disease Study 2019. International Journal of Infectious Diseases 2023, 132, 118-126.
5. Lacy, D. B.; Stevens, R. C. Sequence homology and structural analysis of the clostridial neurotoxins. J Mol Biol 1999, 291, 1091-1104, doi: 10.1006/jmbi.1999.2945.
6. Ovespian, S. V.; Bodeker, M.; O'Leary, V. B.; Lawrence, G. W.; Oliver Dolly, J. Internalization and retrograde axonal trafficking of tetanus toxin in motor neurons and trans-synaptic propagation at central synapses exceed those of its C-terminal-binding fragments. Brain Structure and Function 2015, 220, 1825-1838, doi: 10.1007/s00429-015-1004-0.
7. Megighian, A.; Pirazzini, M.; Fabris, F.; Rossetto, O.; Montecucco, C. Tetanus and tetanus neurotoxin: From peripheral uptake to central nervous tissue targets. J Neurochem 2021, 158, 1244-1253, doi: 10.1111/jnc. 15330.
8. von Bartheld, C. S. Axonal transport and neuronal transcytosis of trophic factors, tracers, and pathogens. J Neurobiol 2004, 58, 295-314, doi: 10.1002/neu. 10315.
9. Schoch, S.; Deak, F.; Konigstorfer, A.; Mozhayeva, M.; Sara, Y.; Sudhof, T. C.; Kavalali, E. T. SNARE function analyzed in synaptobrevin/VAMP knockout mice. Science (New York, N.Y.) 2001, 294, 1117-1122, doi: 10.1126/science.1064335.
10. Schiavo, G.; Rossetto, O.; Tonello, F.; Montecucco, C. Intracellular targets and metalloprotease activity of tetanus and botulism neurotoxins. Curr Top Microbiol Immunol 1995, 195, 257-274, doi: 10.1007/978-3-642-85173-5_12.
11. Albus, U.; Habermann, E. Tetanus toxin inhibits the evoked outflow of an inhibitory (GABA) and an excitatory (D-aspartate) amino acid from particulate brain cortex. Toxicon 1983, 21, 97-110, doi: 10.1016/0041-0101 (83) 90053-3.
12. Rossetto, O.; Montecucco, C. Tables of Toxicity of Botulinum and Tetanus Neurotoxins. Toxins 2019, 11, doi: 10.3390/toxins11120686.
13. Haim M. Solomon, T. L. J. BAM Chapter 17: Clostridium botulinum 8th ed.; 1998, edited 2017
14. Davies, J. R.; Britton, A.; Liu, S. M.; Acharya, K. R. High-resolution crystal structures of the botulinum neurotoxin binding domains from subtypes A5 and A6. FEBS Open Bio 2020, 10, 1474-1481, doi: 10.1002/2211-5463.12931.
15. Hill, K. K.; Smith, T. J.; Helma, C. H.; Ticknor, L. O.; Foley, B. T.; Svensson, R. T.; Brown, J. L.; Johnson, E. A.; Smith, L. A.; Okinaka, R. T., et al. Genetic diversity among Botulinum Neurotoxin-producing clostridial strains. J Bacteriol 2007, 189, 818-832, doi: 10.1128/JB.01180-06.
16. Fischer, A.; Montal, M. Single molecule detection of intermediates during botulinum neurotoxin translocation across membranes. Proceedings of the National Academy of Sciences of the United States of America 2007, 104, 10447-10452, doi: 10.1073/pnas.0700046104.
17. Dong, M.; Stenmark, P. The Structure and Classification of Botulinum Toxins. Handb Exp Pharmacol 2021, 263, 11-33, doi: 10.1007/164_2019_342.
18. Binz, T. Clostridial neurotoxin light chains: devices for SNARE cleavage mediated blockade of neurotransmission. Current topics in microbiology and immunology 2013, 364, 139-157, doi: 10.1007/978-3-642-33570-9_7.
19. Brunger, A. T.; Breidenbach, M. A.; Jin, R.; Fischer, A.; Santos, J. S.; Montal, M. Botulinum neurotoxin heavy chain belt as an intramolecular chaperone for the light chain. PLOS Pathog 2007, 3, 1191-1194, doi: 10.1371/journal.ppat. 0030113.
20. Hill, K. K.; Smith, T. J. Genetic Diversity Within Clostridium botulinum Serotypes, Botulinum Neurotoxin Gene Clusters and Toxin Subtypes; Springer, Berlin, Heidelberg: 2012.
21. Gu, S.; Rumpel, S.; Zhou, J.; Strotmeier, J.; Bigalke, H.; Perry, K.; Shoemaker, C. B.; Rummel, A.; Jin, R. Botulinum neurotoxin is shielded by NTNHA in an interlocked complex. Science (New York, N.Y.) 2012, 335, 977-981, doi: 10.1126/science. 1214270.
22. Sugii, S.; Ohishi, I.; Sakaguchi, G. Correlation between oral toxicity and in vitro stability of Clostridium botulinum type A and B toxins of different molecular sizes. Infect Immun 1977, 16, 910-914, doi: 10.1128/iai.16.3.910-914.1977.
23. Hill, K. K.; Xie, G.; Foley, B. T.; Smith, T. J.; Munk, A. C.; Bruce, D.; Smith, L. A.; Brettin, T. S.; Detter, J. C. Recombination and insertion events involving the botulinum neurotoxin complex genes in Clostridium botulinum types A, B, E and F and Clostridium butyricum type E strains. BMC Biol 2009, 7, 66, doi: 10.1186/1741-7007-7-66.
24. Gu, S.; Jin, R. Assembly and function of the botulinum neurotoxin progenitor complex. Curr Top Microbiol Immunol 2013, 364, 21-44, doi: 10.1007/978-3-642-33570-9_2.
25. Benefield, D. A.; Dessain, S. K.; Shine, N.; Ohi, M. D.; Lacy, D. B. Molecular assembly of botulinum neurotoxin progenitor complexes. P Natl Acad Sci USA 2013, 110, 5630-5635, doi: 10.1073/pnas.1222139110.
26. Marvaud, J. C.; Eisel, U.; Binz, T.; Niemann, H.; Popoff, M. R. TetR is a positive regulator of the tetanus toxin gene in Clostridium tetani and is homologous to botR. Infect Immun 1998, 66, 5698-5702, doi: 10.1128/IAI.66.12.5698-5702.1998.
27. Raffestin, S.; Dupuy, B.; Marvaud, J. C.; Popoff, M. R. BotR/A and TetR are alternative RNA polymerase sigma factors controlling the expression of the neurotoxin and associated protein genes in Clostridium botulinum type A and Clostridium tetani. Molecular microbiology 2005, 55, 235-249, doi: 10.1111/j. 1365-2958.2004.04377.x.
28. Chapeton-Montes, D.; Plourde, L.; Bouchier, C.; Ma, L.; Diancourt, L.; Criscuolo, A.; Popoff, M. R.; Bruggemann, H. The population structure of Clostridium tetani deduced from its pan-genome. Sci Rep 2019, 9, 11220, doi: 10.1038/s41598-019-47551-4.
29. Mueller, J. H.; Miller, P. A. Factors influencing the production of tetanal toxin. J Immunol 1947, 56, 143-147.
30. Heller, H. H. Mutations in the Genus Nicolaierillus (B. Tetani): Studies on Pathogenic Anaerobes VIII. The Journal of Infectious Diseases 1922, 30, 33-43.
31. Cohen, J. E.; Wang, R.; Shen, R. F.; Wu, W. W.; Keller, J. E. Comparative pathogenomics of Clostridium tetani. PLOS One 2017, 12, e0182909, doi: 10.1371/journal.pone.0182909.
32. Schoch C L, C. S., Domrachev M, Hotton C L, Kannan S, Khovanskaya R, Leipe D, Mcveigh R, O'Neill K, Robbertse B, Sharma S, Soussov V, Sullivan J P, Sun L, Turner S, Karsch-Mizrachi I. NCBI Taxonomy: a comprehensive update on curation, resources and tools. Clostridium tetani ATCC 454. January 1 ed.; National Center for Biotechnology Information: 2020; 10.1093/database/baaa062.
33. Woods, C.; Humphreys, C. M.; Rodrigues, R. M.; Ingle, P.; Rowe, P.; Henstra, A. M.; Köpke, M.; Simpson, S. D.; Winzer, K.; Minton, N. P. A novel conjugal donor strain for improved DNA transfer into Clostridium spp. Anaerobe 2019, 59, 184-191, doi: 10.1016/j.anaerobe.2019.06.020.
34. Purdy, D.; O'Keeffe, T. A.; Elmore, M.; Herbert, M.; McLeod, A.; Bokori-Brown, M.; Ostrowski, A.; Minton, N. P. Conjugative transfer of clostridial shuttle vectors from Escherichia coli to Clostridium difficile through circumvention of the restriction barrier. Mol Microbiol 2002, 46, 439-452, doi: 10.1046/j. 1365-2958.2002.03134.x.
35. Heap, J. T.; Pennington, O. J.; Cartman, S. T.; Minton, N. P. A modular system for Clostridium shuttle plasmids. J Microbiol Methods 2009, 78, 79-85, doi: S0167-7012 (09) 00129-8 [pii] 10.1016/j.mimet.2009.05.004 [doi].
36. Garnier, T.; Cole, S. T. Identification and molecular genetic analysis of replication functions of the bacteriocinogenic plasmid pIP404 from Clostridium perfringens. Plasmid 1988, 19, 151-160, doi: 10.1016/0147-619x (88) 90053-4.
37. Girbal, L.; Mortier-Barrière, I.; Raynaud, F.; Rouanet, C.; Croux, C.; Soucaille, P. Development of a sensitive gene expression reporter system and an inducible promoter-repressor system for Clostridium acetobutylicum. Appl Environ Microbiol 2003, 69, 4985-4988, doi: 10.1128/aem.69.8.4985-4988.2003.
38. Graves, M. C.; Mullenbach, G. T.; Rabinowitz, J. C. Cloning and nucleotide sequence determination of the Clostridium pasteurianum ferredoxin gene. Proc Natl Acad Sci USA 1985, 82, 1653-1657, doi: 10.1073/pnas.82.6.1653.
39. Chapeton-Montes, D.; Plourde, L.; Deneve, C.; Garnier, D.; Barbirato, F.; Colombié, V.; Demay, S.; Haustant, G.; Gorgette, O.; Schmitt, C., et al. Tetanus Toxin Synthesis is Under the Control of A Complex Network of Regulatory Genes in Clostridium tetani. Toxins 2020, 12, 328.
40. Latham, W. C.; Bent, D. F.; Levine, L. Tetanus toxin production in the absence of protein. Appl Microbiol 1962, 10, 146-152, doi: 10.1128/am.10.2.146-152.1962.
41. Demain, A. L.; Gerson, D. F.; Fang, A. Effective levels of tetanus toxin can be made in a production medium totally lacking both animal (e.g., brain heart infusion) and dairy proteins or digests (e.g., casein hydrolysates). Vaccine 2005, 23, 5420-5423, doi: 10.1016/j.vaccine.2005.03.043.
42. Demain, A. L.; George, S.; Kole, M.; Gerson, D. F.; Fang, A. Tetanus toxin production in soy-based medium: nutritional studies and scale-up into small fermentors. Lett Appl Microbiol 2007, 45, 635-638, doi: 10.1111/j. 1472-765X.2007.02238.x.
43. Demain, A. L.; Gerson, D. F.; Kole, M.; Fang, A. The role of reduced iron powder in the fermentative production of tetanus toxin. Applied microbiology and biotechnology 2006, 73, 55-59, doi: 10.1007/s00253-006-0450-2.

44 Popoff, M. R.; Brüggemann, H. Regulatory Networks Controlling Neurotoxin Synthesis in Clostridium botulinum and Clostridium tetani. Toxins 2022, 14, doi: 10.3390/toxins14060364.
45. Pennings, J. L. A.; Abachin, E.; Esson, R.; Hodemaekers, H.; Francotte, A.; Claude, J. B.; Vanhee, C.; Uhlrich, S.; Vandebriel, R. J. Regulation of Clostridium tetani Neurotoxin Expression by Culture Conditions. Toxins (Basel) 2022, 14, doi: 10.3390/toxins14010031.
46. Bradshaw, M.; Marshall, K. M.; Heap, J. T.; Tepp, W. H.; Minton, N. P.; Johnson, E. A. Construction of a nontoxigenic Clostridium botulinum strain for food challenge studies. Appl Environ Microbiol 2010, 76, 387-393, doi: 10.1128/AEM.02005-09.
47. Inzalaco, H. N.; Tepp, W. H.; Fredrick, C.; Bradshaw, M.; Johnson, E. A.; Pellett, S. Posttranslational Regulation of Botulinum Neurotoxin Production in Clostridium botulinum Hall A-hyper. mSphere 2021, 6, e0032821, doi: 10.1128/mSphere.00328-21.
48. Bradshaw, M.; Tepp, W. H.; Whitemarsh, R. C.; Pellett, S.; Johnson, E. A. Holotoxin Activity of Botulinum Neurotoxin Subtype A4 Originating from a Nontoxigenic Clostridium botulinum Expression System. Appl Environ Microbiol 2014, 80, 7415-7422, doi: 10.1128/aem.01795-14.
49. Demain, A.; Gerson, D.; Kole, M.; Fang, A. The role of reduced iron powder in the fermentative production of tetanus toxin. Applied microbiology and biotechnology 2006, 73, 55-59, doi: 10.1007/s00253-006-0450-2.
50. Eric A. Johnson, M. B., Julian I. Rood, Dena Lyras Expression system for clostridium species. 1998.
51. Chen, F.; Kuziemko, G. M.; Amersdorfer, P.; Wong, C.; Marks, J. D.; Stevens, R. C. Antibody mapping to domains of botulinum neurotoxin serotype A in the complexed and uncomplexed forms. Infect Immun 1997, 65, 1626-1630, doi: 10.1128/iai.65.5.1626-1630.1997.
52. Ohishi, I.; Sugii, S.; Sakaguchi, G. Oral toxicities of Clostridium botulinum toxins in response to molecular size. Infect Immun 1977, 16, 107-109, doi: 10.1128/iai.16.1.107-109.1977.
53. Mueller, J. H.; Miller, P. A. Variable factors influencing the production of tetanus toxin. J Bacteriol 1954, 67, 271-277, doi: 10.1128/jb.67.3.271-277.1954.
54. Ingle, P.; Groothuis, D.; Rowe, P.; Huang, H.; Cockayne, A.; Kuehne, S. A.; Jiang, W.; Gu, Y.; Humphreys, C. M.; Minton, N. P. Generation of a fully erythromycin-sensitive strain of Clostridioides difficile using a novel CRISPR-Cas9 genome editing system. Sci Rep 2019, 9, 8123, doi: 10.1038/s41598-019-44458-y.
55. Licona-Cassani, C.; Steen, J. A.; Zaragoza, N. E.; Moonen, G.; Moutafis, G.; Hodson, M. P.; Power, J.; Nielsen, L. K.; Marcellin, E. Tetanus toxin production is triggered by the transition from amino acid consumption to peptides. Anaerobe 2016, 41, 113-124, doi: 10.1016/j.anaerobe.2016.07.006.
56. Chung, Y.-J.; Jung, M.-Y.; Lee, J.-A.; Kim, T.-Y.; Choe, Y.-K.; Kim, I.-H. Tetanus toxin production from Clostridium tetani, using a casein-based medium in a single-use bioreactor. Biotechnology and Bioprocess Engineering 2016, 21, 531-536, doi: 10.1007/s12257-016-0355-6.
57. Fratelli, F.; Siquini, T. J.; Prado, S. M.; Higashi, H. G.; Converti, A.; de Carvalho, J. C. Effect of medium composition on the production of tetanus toxin by Clostridium tetani. Biotechnology progress 2005, 21, 756-761, doi: 10.1021/bp049571b.
58. de Marco, A. Strategies for successful recombinant expression of disulfide bond-dependent proteins in Escherichia coli. Microb Cell Fact 2009, 8, 26, doi: 10.1186/1475-2859 Aug. 26.
59. Viravathana, P.; Tepp, W. H.; Bradshaw, M.; Przedpelski, A.; Barbieri, J. T.; Pellett, S. Potency Evaluations of Recombinant Botulinum Neurotoxin A1 Mutants Designed to Reduce Toxicity. International journal of molecular sciences 2024, 25, doi: 10.3390/ijms25168955.
60. Rigoni, M.; Caccin, P.; Johnson, E. A.; Montecucco, C.; Rossetto, O. Site-Directed Mutagenesis Identifies Active-Site Residues of the Light Chain of Botulinum Neurotoxin Type A. Biochemical and Biophysical Research Communications 2001, 288, 1231-1237.
61. Marshall, K. M.; Bradshaw, M.; Johnson, E. A. Conjugative botulinum neurotoxin-encoding plasmids in Clostridium botulinum. PloS one 2010, 5, e11087, doi: 10.1371/journal.pone.0011087.
62. Przedpelski, A.; Tepp, W. H.; Pellett, S.; Johnson, E. A.; Barbieri, J. T. A Novel High-Potency Tetanus Vaccine. mBio 2020, 11, doi: 10.1128/mBio.01668-20.
63. Malizio, C. J.; Goodnough, M. C.; Johnson, E. A. Purification of Clostridium botulinum type A neurotoxin. Methods Mol Biol 2000, 145, 27-39, doi: 10.1385/1-59259-052-7:27.

### Sequences

| SEQ ID | |
|---|---|
| NO | Description |
| 1 | Ndel-tent primer |
| 2 | tent-Nhel primer |
| 3 | ferredoxin |
| 4 | thiolase |
| 5 | tent |
| 6 | catp cloramphenicol resistance |
| 7 | A1 CAL82360.1 botulinum neurotoxin type A precursor [Clostridium botulinum A |
| | str. ATCC 3502] |
| 8 | A2 CAA51824.1 botulinum neurotoxin type A [Clostridium botulinum A2 str. |
| | Kyoto] |
| 9 | A3 ACA57525.1 bontoxilysin A (plasmid) [Clostridium botulinum A3 str. Loch |
| | Maree] |
| 10 | A4 ACQ51417.1 botulinum neurotoxin type BvA4, BoNT/BvA4 (plasmid) |
| | [Clostridium botulinum Ba4 str. 657] |
| 11 | A5 ACG50065.1 botulinum neurotoxin sub-type A5 [Clostridium botulinum |
| | H04402 0651 |
| 12 | A6 ACW83608.1 botulinum neurotoxin type A [Clostridium botulinum] |
| 13 | A7 AFV13854.1 botulinum neurotoxin [Clostridium botulinum] |
| 14 | A8 AJA05787.1 BoNT/A8 [Clostridium botulinum A] |
| 15 | B1 ACA46990.1 bontoxilysin A (plasmid) [Clostridium botulinum B1 str. Okra] |
| 16 | B2 BAC22064.1 neurotoxin (plasmid) [Clostridium botulinum] |
| 17 | B3 ABM73977.1 neurotoxin B [Clostridium botulinum] |
| 18 | B4 ABM73987.1 neurotoxin B [Clostridium botulinum B str. Eklund 17B (NRP)] |
| 19 | B5 ACQ51206.1 botulinum neurotoxin type BvB, BoNT/BvB (plasmid) |
| | [Clostridium botulinum Ba4 str. 657] |
| 20 | B6 BAF91946.1 neurotoxin type B [Clostridium botulinum] |
| 21 | B7 AFD33678.1 botulinum neurotoxin B subtype/variant B7 [Clostridium |
| | botulinum] |
| 22 | B8 AFN61309.1 neurotoxin B8 [Clostridium botulinum] |
| 23 | C1 BAA14235.1 botulinum C1 neurotoxin [Clostridium phage c-st] |
| 24 | CD BAA08418.1 neurotoxin [Clostridium botulinum C] |
| 25 | D1 EES90380.1 botulinum neurotoxin type D, BoNT/D [Clostridium phage D- |
| | 1873] |
| 26 | DC ABP48747.1 neurotoxin [Clostridium botulinum] |
| 27 | E1 CAA43999.1 botulinum neurotoxin type E [Clostridium botulinum] |
| 28 | E2 ABM73981.1 neurotoxin E [Clostridium botulinum] |
| 29 | E3 ABM73980.1 neurotoxin E [Clostridium botulinum] |
| 30 | E4 BAC05434.1 type E botulinum toxin [Clostridium butyricum] |
| 31 | E5 BAB03512.1 type E botulinum toxin [Clostridium butyricum] |
| 32 | E6 CAM91125.1 botulinum neurotoxin [Clostridium botulinum E] |
| 33 | E7 AER11391.1 botulinum neurotoxin E [Clostridium botulinum] |
| 34 | E8 AER11392.1 botulinum neurotoxin E [Clostridium botulinum] |
| 35 | E9 AFV91339.1 botulinum neurotoxin type E [Clostridium botulinum] |
| 36 | E10 AII82330.1 botulinum neurotoxin type E variant E10 [Clostridium botulinum] |
| 37 | E11 AII82289.1 botulinum neurotoxin type E variant E11 [Clostridium botulinum] |
| 38 | E12 AHK10119.1 neurotoxin E [Clostridium botulinum] |
| 39 | F1 ABS41202.1 botulinum neurotoxin type F, BoNT/F [Clostridium botulinum F |
| | str. Langeland] |
| 40 | F2 CAA73972.1 bonT [Clostridium botulinum] |
| 41 | F3 ADA79575.1 botulinum neurotoxin type F [Clostridium botulinum] |
| 42 | F4 ADA79569.1 botulinum neurotoxin type F [Clostridium botulinum] |
| 43 | F5 ADA79560.1 botulinum neurotoxin type F [Clostridium botulinum] |
| 44 | F6 AAA23263.1 neurotoxin type F [Clostridium botulinum] |
| 45 | F7 ADK48765.1 botulinum neurotoxin type F [Clostridium baratii] |
| 46 | F8 KEJ01913.1 peptidase M27 [Clostridium botulinum F 357] |
| 47 | G KIE44899.1 botulinum neurotoxin type G [Clostridium argentinense CDC 2741] |
| 48 | FA KGO15617.1 peptidase M27 [Clostridium botulinum] |
| 49 | Wo WP 027699549.1 hypothetical protein [Weissella oryzae] |
| 50 | X BAQ12790.1 putative botulinum neurotoxin [Clostridium botulinum] |
| 51 | TeNT WP 011100836.1 tetanus toxin [Clostridium tetani] |
| 52 | En OTO22244.1 hypothetical protein A5816_002916 [Enterococcus sp. |
| | 3G1 DIV0629] |
| 53 | Cp1 ASW73945.2 hypothetical protein CJF12 06335 [Chryseobacterium piperi] |
| 54 | Cp2 ASW75385.1 hypothetical protein CJF12_14550 [Chryseobacterium piperi] |
| 55 | Cp3 ASW73949.2 hypothetical protein CJF12 06355 [Chryseobacterium piperi] |
| 56 | PMP1 WP 150887772.1 hypothetical protein [Paraclostridium bifermentans] |
| 57 | PGT1 WP 250673407.1 and WP 250673408.1 M91 family zinc metallopeptidase |
| | [Paeniclostridium ghonii] |

What is claimed:

1. An expression vector comprising a promoter operably linked to a polynucleotide encoding a botulinum neurotoxin (BoNT).

2. The expression vector of claim 1, wherein the promoter is a ferredoxin promoter, a thiolase promoter, or a tent promoter.

3. The expression vector of claim 1, wherein the BoNT is type A (BoNT/A), type B (BoNT/B), type C (BoNT/C), type D (BoNT/D), type E (BoNT/E), type F (BoNT/F), or type G (BoNT/G).

4. The expression vector of claim 1, wherein the BoNT is an engineered BONT.

5. An atoxic Clostridium bacterium engineered to express a heterologous BONT.

6. The bacterium of claim 5, wherein the bacterium is an atoxic Clostridium tetani.

7. The bacterium of claim 6, wherein the bacterium is:
a) Clostridium tetani ATCC 454; or
b) Clostridium ATCC 19406.

8. The bacterium of claim 5, wherein the bacterium comprises the expression vector comprising a promoter operably linked to a polynucleotide encoding the heterologous BONT.

9. The bacterium of claim 5, wherein the BoNT is type A (BoNT/A), type B (BoNT/B), type C (BoNT/C), type D (BoNT/D), type E (BoNT/E), type F (BoNT/F), or type G (BoNT/G).

10. The bacterium of claim 5, wherein the BoNT is an engineered BONT.

11. The bacterium of claim 5, wherein the bacterium produces active BONT in the absence of neurotoxin-associated proteins.

12. A method of making the bacterium of claim 5, the method comprising introducing a polynucleotide encoding the BoNT into the bacterium.

13. The method of claim 12, wherein the polynucleotide is introduced via conjugation with a donor strain.

14. A method of producing a BoNT, the method comprising:
a) culturing the bacterium of claim 5 in a culture medium; and
b) collecting the BoNT from the culture.

15. The method of claim 14, wherein the bacterium is cultured for 1-14 days prior to step (b).

16. The method of claim 14, wherein step (b) comprises removing the bacterium from the culture medium to produce a cell-free culture supernatant comprising the BoNT.

17. The method of claim 16, further comprising purifying the BoNT from the cell-free culture supernatant.

18. The method of claim 14, wherein the culture medium is a modified Mueller Miller medium.

19. A BONT made by the method of claim 14.

20. A method of treating a condition in a subject comprising administering the composition of claim 19 to the subject to treat the condition.
